SEQUENCE LISTING (399 sequences)

SEQ ID NO: 1 - protein, 19 aa, Mus musculus
EVQLVESGGDLVKPGGSLK

SEQ ID NO: 2 - protein, 11 aa, Mus musculus
NTLYLQMSSLK

SEQ ID NO: 3 - protein, 10 aa, Mus musculus
LVESGGGLVK

SEQ ID NO: 4 - protein, 11 aa, Mus musculus
NNLYLQMSSLK

SEQ ID NO: 5 - protein, 19 aa, Mus musculus
EVQLVESGGGLVKPGGSLK

SEQ ID NO: 6 - protein, 15 aa, Mus musculus
ASGYTFTDYSMHWVK

SEQ ID NO: 7 - protein, 19 aa, Mus musculus
EVQLQPSGAELVKPGASVK

SEQ ID NO: 8 - protein, 14 aa, Mus musculus
QSGAELVRPGASVK

SEQ ID NO: 9 - protein, 19 aa, Mus musculus
EVQLQPSGAELVKPGASVK

SEQ ID NO: 10 - protein, 11 aa, Mus musculus
LSCTASGFNIK

SEQ ID NO: 11 - protein, 13 aa, Mus musculus
LLESGGGLVQPGR

SEQ ID NO: 12 - DNA, 23 bp, Artificial
  primer sequence for Vgeneric
sadgtbcagctkmagsagtcwgg

SEQ ID NO: 13 - DNA, 23 bp, Artificial
  primer sequence for V3609
carrttaytcwgaaaswgtctgg

SEQ ID NO: 14 - DNA, 23 bp, Artificial
  primer sequence for VS107/J606
gargtgmagctkgwdgarwctgr

SEQ ID NO: 15 - DNA, 23 bp, Artificial
  primer sequence for J558
saggtycarctscarcagyctgg

SEQ ID NO: 16 - DNA, 23 bp, Artificial
  primer sequence for VGAM
cagatccagttsgtrcagtctgg

SEQ ID NO: 17 - DNA, 23 bp, Artificial
  primer sequence for V7183/VH11
gamgtgmagctsktggagwctgg

SEQ ID NO: 18 - DNA, 18 bp, Artificial
  primer sequence for JH1
cggtcaccgtytcctcag

SEQ ID NO: 19 - DNA, 21 bp, Artificial
  primer sequence for JH2
gcaccastctcacagtctcct

SEQ ID NO: 20 - DNA, 21 bp, Artificial
  primer sequence for JH3
gggactctggtcactgtctct

SEQ ID NO: 21 - DNA, 21 bp, Artificial
  primer sequence for JH4
aacctcagtcaccgtctcctc

SEQ ID NO: 22 - DNA, 19 bp, Artificial
  primer sequence for J/hinge
caccgtctcctcagagccc

SEQ ID NO: 23 - DNA, 20 bp, Artificial
  primer sequence for gammaCH2a
tgttgaccytgcatttgaac

SEQ ID NO: 24 - DNA, 21 bp, Artificial
  primer sequence for gamme CH2b
ttkgagatggttytctcgatg

SEQ ID NO: 25 - DNA, 22 bp, Artificial
  primer sequence for gamma CH2c
gttgaccttgcatttgaactcc

SEQ ID NO: 26 - DNA, 25 bp, Artificial
  primer sequence for gamma CH2d
ttggagggaagatgaagacggatgg

SEQ ID NO: 27 - DNA, 28 bp, Artificial
  primer sequence for gamma CH2e
tgttgaccytgcatttgaactccttgcc

SEQ ID NO: 28 - DNA, 18 bp, Artificial
  primer sequence for beta-actin 2
gatatcgctgcgctggtc

SEQ ID NO: 29 - DNA, 20 bp, Artificial
  primer sequence for beta-actin 4
ctacgtacatggctggggtg

SEQ ID NO: 30 - DNA, 24 bp, Artificial
  primer sequence for VDJ029
cggggggctacggctacgtatggg

SEQ ID NO: 31 - DNA, 25 bp, Artificial
  primer sequence for JH4long
ggaacctcagtcaccgtctcctcag

SEQ ID NO: 32 - DNA, 29 bp, Artificial
  primer sequence for gamma2bhingelong
agtgacttacctgggcatttgtgacactc

SEQ ID NO: 33 - DNA, 20 bp, Artificial
  primer sequence for gamma2aCH2long
agggcactgaccacccggag

SEQ ID NO: 34 - DNA, 24 bp, Artificial
  primer sequence for 3′Emu
gacctctccgaaaccaggcaccgc

SEQ ID NO: 35 - DNA, 11 bp, Mus musculus
tgtgtgagaca

SEQ ID NO: 36 - DNA, 10 bp, Mus musculus
tgtgcaagag

SEQ ID NO: 37 - DNA, 11 bp, Mus musculus
tgtgtgagaca

SEQ ID NO: 38 - DNA, 11 bp, Mus musculus
tgtgcaagaca

SEQ ID NO: 39 - DNA, 10 bp, Mus musculus
cgccgggggg

SEQ ID NO: 40 - DNA, 16 bp, Mus musculus
ctactatgattacgac

SEQ ID NO: 41 - DNA, 10 bp, Mus musculus
ctacggctac

SEQ ID NO: 42 - DNA, 13 bp, Mus musculus
tactatgattacg

SEQ ID NO: 43 - DNA, 19 bp, Mus musculus
ttactacggtgatacctac

SEQ ID NO: 44 - DNA, 15 bp, Mus musculus
tctactttgattacg

SEQ ID NO: 45 - DNA, 12 bp, Mus musculus
ggtggtaactac

SEQ ID NO: 46 - DNA, 18 bp, Mus musculus
tatgctatggactactgg

SEQ ID NO: 47 - DNA, 18 bp, Mus musculus
tatgctatggactactgg

SEQ ID NO: 48 - DNA, 14 bp, Mus musculus
actttgactactgg

SEQ ID NO: 49 - DNA, 18 bp, Mus musculus
tatgcgacggactactgg

SEQ ID NO: 50 - DNA, 12 bp, Mus musculus
atggactactgg

SEQ ID NO: 51 - DNA, 13 bp, Mus musculus
ctttgactactgg

SEQ ID NO: 52 - DNA, 11 bp, Mus musculus
ctcctaacctc

SEQ ID NO: 53 - DNA, 17 bp, Artificial
  primer sequence for 3′Emu
gcactgaccacccggag

SEQ ID NO: 54 - DNA, 20 bp, Artificial
  primer sequence for Calpha3
gctcctttaggggctcaaac

SEQ ID NO: 55 - DNA, 20 bp, Artificial
  primer sequence for Calpha2L1
caggcaggacgctggacaca

SEQ ID NO: 56 - DNA, 20 bp, Artificial
  primer sequence for Calpha2L2
actgtagcagccgcaggaat

SEQ ID NO: 57 - protein, 22 aa, Mus musculus
PALEDLLLGSDASITCTLNGLR

SEQ ID NO: 58 - protein, 20 aa, Mus musculus
NPEGAVFTWEPSTGKDAVQK

SEQ ID NO: 59 - protein, 22 aa, Mus musculus
KAVQNSCGCYSVSSVLPGCAER

SEQ ID NO: 60 - protein, 8 aa, Mus musculus
WNSGASFK

SEQ ID NO: 61 - protein, 17 aa, Mus musculus
CTVTHPESGTLTGTIAK

SEQ ID NO: 62 - protein, 7 aa, Mus musculus
VSAETWK

SEQ ID NO: 63 - protein, 21 aa, Mus musculus
QGDQYSCMVGHEALPMNFTQK

SEQ ID NO: 64 - protein, 22 aa, Mus musculus
LSGKPTNVSVSVIMSEGDGICY

SEQ ID NO: 65 - protein, 10 aa, Mus musculus
AFNPKEVLVR

SEQ ID NO: 66 - protein, 16 aa, Mus musculus
EPGEGATTYLVTSVLR

SEQ ID NO: 67 - protein, 33 aa, Mus musculus
VTVNTFPPQVHLLPPPSEELALNELLSLTCLVR

SEQ ID NO: 68 - protein, 16 aa, Mus musculus
LVESGGGLVKPGGSLK

SEQ ID NO: 69 - protein, 13 aa, Mus musculus
EVQLVESGGGLVK

SEQ ID NO: 70 - protein, 19 aa, Mus musculus
EVQLVESGGGLVKPGGSLK

SEQ ID NO: 71 - protein, 19 aa, Mus musculus
EVKLVESGGGLVQPGGSLK

SEQ ID NO: 72 - protein, 19 aa, Mus musculus
EVKLVESGGGLVKPGGSLK

SEQ ID NO: 73 - protein, 11 aa, Mus musculus
NTLYLQMSSLK

SEQ ID NO: 74 - protein, 11 aa, Mus musculus
NTLYLQMNSLK

SEQ ID NO: 75 - protein, 11 aa, Mus musculus
NNLYLQMSSLK

SEQ ID NO: 76 - protein, 11 aa, Mus musculus
NILYLQMSSLR

SEQ ID NO: 77 - protein, 11 aa, Mus musculus
SEDTAMYYCAR

SEQ ID NO: 78 - protein, 19 aa, Mus musculus
LSCAASGFAFSSYDMSWVR

SEQ ID NO: 79 - protein, 22 aa, Mus musculus
RLEWVAYISSGGGSTYYPDTVK

SEQ ID NO: 80 - protein, 7 aa, Mus musculus
ATLTVDK

SEQ ID NO: 81 - protein, 19 aa, Mus musculus
EVQLQQSGPELVKPGASVK

SEQ ID NO: 82 - protein, 13 aa, Mus musculus
QLKLQESGPELVK

SEQ ID NO: 83 - protein, 19 aa, Mus musculus
QVQLQQSGPELVKPGASVK

SEQ ID NO: 84 - protein, 19 aa, Mus musculus
  misc_feature (7)..(7): Xaa can be any naturally occurring amino acid
QVQLQQXGAELVKPGASVK

SEQ ID NO: 85 - protein, 7 aa, Mus musculus
SLEWIGR

SEQ ID NO: 86 - protein, 20 aa, Mus musculus
SLEWIGDINPNNGGTSYNQK

SEQ ID NO: 87 - protein, 12 aa, Mus musculus
ASGYTFTDYYMK

SEQ ID NO: 88 - protein, 13 aa, Mus musculus
QIQLVQSGPELKK

SEQ ID NO: 89 - protein, 12 aa, Mus musculus
QIQLVQSGPELK

SEQ ID NO: 90 - protein, 19 aa, Mus musculus
QIQLVQSGPELKKPGETVK

SEQ ID NO: 91 - protein, 11 aa, Mus musculus
SEDTATYFCAR

SEQ ID NO: 92 - protein, 6 aa, Mus musculus
NQFFLK

SEQ ID NO: 93 - protein, 6 aa, Mus musculus
YNPSLK

SEQ ID NO: 94 - protein, 159 aa, Mus musculus
MKTHLLLWGVLAIFVKAVLVTGDDEATILADNKCMCTRVTSRIIPSTEDPNEDIVERNIRIVVPLNNRENISDPTSPLRRNFVYHLSDVCKKCDPVEVELEDQVVTATQSNICNEDDGVPETCYMYDRNKCYTTMVPLRYHGETKMVQAALTPDSCYPD

SEQ ID NO: 95 - DNA, 302 bp, Mus musculus
gaggtgcagcttgttgagtctggtggaggattggtgcagcctaaagggtcattgaaactctcatgtgcagcctctggattcagcttcaatacctacgccatgaactgggtccgccaggctccaggaaagggtttggaatgggttgctcgcataagaagtaaaagtaataattatgcaacatattatgccgattcagtgaaagacagattcaccatctccagagatgattcagaaagcatgctctatctgcaaatgaacaacttgaaaactgaggacacagccatgtattactgtgtgagaca

SEQ ID NO: 96 - DNA, 302 bp, Mus musculus
gaggtgcagcttgttgagtctggtggaggattggtgcagcctacagggtcattgaaactctcatgtgcagcctctggattcatgttcaatacctacgccatgaactgggtccgccaggctccaggaaagggtttggagtggcttgctcgcataagaactaaaagtaataattatgcaacatattatgccgattcagtgaaggaccggttcaccatatccagagatgattcacagaccatgctctatctgcaaatgaacaacttgaaaactgaggacacagccttgtattactgtgtgagaca

SEQ ID NO: 97 - DNA, 279 bp, Mus musculus
tggaggattggtgcagcctaaagggtcattgaaactctcatgtgcagcctctggattcaccttcaatacctacgccatgaactgggtccgccaggctccaggaaagggtttggaatgggttgctcgcataagaagtaaaagtaataattatgcaacatattatgccgattcagtgaaagacaggttcaccatctccagagatgattcacaaagcatgctctatctgcaaatgaacaacttgaaaactgaggacacagccatgtattactgtgtgagaca

SEQ ID NO: 98 - DNA, 296 bp, Mus musculus
gaggtgcagctggtggagtctgggggaggcttagtgcagcctggagggtccctgaaactctcctgtgcagcctctggattcactttcagtagctatggcatgtcttgggttcgccagactccagacaagaggctggagttggtcgcaaccattaatagtaatggtggtagcacctattatccagacagtgtgaagggccgattcaccatctccagagacaatgccaagaacaccctgtacctgcaaatgagcagtctgaagtctgaggacacagccatgtattactgtgcaagaga

SEQ ID NO: 99 - DNA, 296 bp, Mus musculus
gaggtgcagttggtggagtctgggggaggcttagtgcagcctggagggtccctgaaactctcctgtgcagcctctggattcacttttagtagttatggcatgtcttgggttcgccagactccagacaagaggctggagtgggtcgcaagcatcaatagtaatggtggtagcaccgactatacagacagtgtgaagggccgattcaccatctccagagacaatgccaagaacaccctgtatctgcaaatgaggagtctgaagtctgaggacacagccagatattactgtgcaagagc

SEQ ID NO: 100 - DNA, 294 bp, Mus musculus
caggtccagctgcagcagtctggacctgagctggtgaagcctggggcttcagtgaagatatcctgcaaggcttctggctacagcttcacaagctactatatacactgggtgaagcagaggcctggacagggacttgagtggattggatggatttatcctggaagtggtaatactaagtacaatgagaagttcaagggcaaggccacactgacggcagacacatcctccagcactgcctacatgcagctcagcagcctaacatctgaggactctgcggtctattactgtgcaaga

SEQ ID NO: 101 - DNA, 294 bp, Mus musculus
caggtccagctgcagcagtctggacctgagctggtgaagcctggggcttcagtgaagatatcctgcaaggcttctggctacagcttcacaagctactatatacactgggtgaagcagaggcctggacagggacttgagtggattggatggatttttcctggaagtggtaatactaagtacaatgagaagttcaagggcaaggccacactgacggcagacacatcctccagcacagcctacatgcagctcagcagcctgacatctgaggactctgcagtctatttctgtgcccga

SEQ ID NO: 102 - DNA, 296 bp, Mus musculus
gaggtgcagctggtggagtctgggggagacttagtgaagcctggagggtccctgaaactctcctgtgcagcctctggattcactttcagtagctatggcatgtcttgggttcgccagactccagacaagaggctggagtgggtcgcaaccattagtagtggtggtagttacacctactatccagacagtgtgaaggggcgattcaccatctccagagacaatgccaagaacaccctgtacctgcaaatgagcagtctgaagtctgaggacacagccatgtattactgtgcaagaca

SEQ ID NO: 103 - DNA, 273 bp, Mus musculus
gggagacttagtgaagcctggagggtccctgaaactctcctgtgcagcctctggattcactttcagtagctatggcatgtcttgggttcgccagactccagacaagaggctggagtgggtcgcaaccattagtagtggtggtcgttacacctactatccagacagtgtgaagggacgattcaccatctccagagacaatgccaagaacaccctgtacctgcaaatgagcagtctgaagtctgaggacacagccatgtattactgtgcaagaca

SEQ ID NO: 104 - DNA, 300 bp, Mus musculus
gaagtgaagcttgaggagtctggaggaggcttggtgcaacctggaggatccatgaaactctcctgtgttgcctctggattcactttcagtaactactggatgaactgggtccgccagtctccagagaaggggcttgagtgggttgctgaaattagattgaaatctaataattatgcaacacattatgcggagtctgtgaaagggaggttcaccatctcaagagatgattccaaaagtagtgtctacctgcaaatgaacaacttaagagctgaagacactggcatttattactgtaccagg

SEQ ID NO: 105 - DNA, 277 bp, Mus musculus
aggaggcttggtgcaacctggaggatccatgaaactctcctgtgttgcctctggattcactttcagtaactactggatgaactgggtccgccagtctccagagaaggggcttgagtgggttgctgaaattagattgaaatctaataattatgcaacacattatgcggagtctgtgaaagggaggttcaccatctcaagagatgattccaaaagtagtgtctacctgcaaatgaacaacttaagagctgaagacactggcatttattactgtaccagg

SEQ ID NO: 106 - DNA, 294 bp, Mus musculus
cagatccagttggtgcagtctggacctgagctgaagaagcctggagagacagtcaagatctcctgcaaggcttctgggtataccttcacaaactatggaatgaactgggtgaagcaggctccaggaaagggtttaaagtggatgggctggataaacaccaacactggagagccaacatatgctgaagagttcaagggacggtttgccttctctttggaaacctctgccagcactgcctatttgcagatcaacaacctcaaaaatgaggacacggctacatatttctgtgcaaga

SEQ ID NO: 107 - DNA, 271 bp, Mus musculus
acctgagctgaagaagcctggagagacagtcaagatctcctgcaaggcttctgggtataccttcacaaactatggaatgagctgggtgaagcagactccaggaaagggtttaaagtggatgggctggataaacaccaacactggagagcgaacatatgctgaagatttcaagggacggtttgccttctctttggaaacctctgccagcactgcctatttgcagatcaacaacctcaaaaatgaggacacggctacatatttctgtgcaaga

SEQ ID NO: 108 - DNA, 294 bp, Mus musculus
gaggttcagctgcagcagtctggggcagagcttgtgaggtcaggggcctcagtcaagttgtcctgcacagcttctggcttcaacattaaagactactatatgcactgggtgaagcagaggcctgaacagggcctggagtggattggatggattgatcctgagaatggtgatactgaatatgccccgaagttccagggcaaggccactatgactgcagacacatcctccaacacagcctacctgcagctcagcagcctgacatctgaggacactgccgtctattactgtaatgca

SEQ ID NO: 109 - DNA, 271 bp, Mus musculus
ggcagagcttgtgaggtcaggggcctcagtcaagttgtcctgcacagcttctggcttcaacattaaagaccactatatgcactgggtgaagcagaggcctgaacagggcctggagtggattggatggattgatcctgagaatggtgataatgaatatgccccgaagttccagggcaaggccactatgactgcagacacatcctccaacacagcctacctgcagctcagcagcctgacatctgaggacactgccgtctattactgtaatgca

SEQ ID NO: 110 - DNA, 294 bp, Mus musculus
caggttcagctgcagcagtctggagctgagctgatgaagcctggggcctcagtgaagctttcctgcaaggctactggctacacattcactggctactggatagagtgggtaaagcagaggcctggacatggccttgagtggattggagagattttacctggaagtggtagtactaactacaatgagaagttcaagggcaaggccacattcactgcagatacatcctccaacacagcctacatgcaactcagcagcctgacaactgaggactctgccatctattactgtgcaaga

SEQ ID NO: 111 - DNA, 271 bp, Mus musculus
agctgagctgatgaagcctggggcctcagtgaagatatcctgcaaggctactggctacacattcagtagctactggatagagtgggtaaagcagaggcctggacatggccttgagtggattggagagattttacctggaagtggtagtactaactacaatgagaagttcaagggcaaggccacattcactgcagatacatcctccaacacagcctacatgcaactcagcagcctgacatctgaggactctgccgtctattactgtgcaaga

SEQ ID NO: 112 - DNA, 640 bp, Mus musculus
cagccaaaacaacacccccatcagtctatccactggcccctgggtgtggagatacaactggttcctccgtgactctgggatgcctggtcaagggctacttccctgagtcagtgactgtgacttggaactctggatccctgtccagcagtgtgcacaccttcccagctctcctgcagtctggactctacactatgagcagctcagtgactgtcccctccagcacctggccaagtcagaccgtcacctgcagcgttgctcacccagccagcagcaccacggtggacaaaaaacttggtgagaggacattcaggggaggagggattcaccagagttgaggcaaaggtattagcctatctaaaccagccaggctgggatccatcaccaaggaggtgaccttagcccagggaagagggagatactgtctctgcctccctcctgggaacatctagctatgaccacctacactcaaggacatgatcctctgggataggtgtgcttgtcatttccaggatcatcctggactaagcccataccagggacaaactttcctctctctggtttggtgcttctctccttcaaaaaccagtaacatccagccttctctctgcagagcccagcgggcccatttcaacaatcaacc

SEQ ID NO: 113 - DNA, 566 bp, Mus musculus
ctgggatgcctggtcaagggctacttccctgagtcagtgactgtgacttggaactctggatccctgtccagcagtgtgcacaccttcccagctctcctgcagtctggactctacactatgagcagctcagtgactgtcccctccagcacctggccaagtcagaccgtcacctgcagcgttgctcacccagccagcagcaccacggtggacaaaaaacttggtgagaggacattcaggggaggagggattcaccagagttgaggcaaaggtattagcctatctaaaccagccaggctgggatccatcaccaaggaggtgaccttagcccagggaagagggagatactgtctctgcctccctcctgggaacatctagctatgaccacctacactcaaggacatgatcctctgggataggtgtgcttgtcatttccaggatcatcctggactaagcccataccagggacaaactttcctctctctggtttggtgcttctctccttcaaaaaccagtaacatccagccttctctctgcagagcccagcgggcccatttcaacaatcaacc

SEQ ID NO: 114 - DNA, 188 bp, Mus musculus
tgaccacctacactcaaggacatgatcctctgggataggtgtgcttgtcatttccaggatcatcctggactaagcccataccagggacaaactttcctctctctggtttggtgcttctctccttcaaaaaccagtaacatccagccttctctctgcagagcccagcgggcccatttcaacaatcaacc

SEQ ID NO: 115 - DNA, 137 bp, Mus musculus
ttccaggatcatcctggactaagcccataccagggacaaactttcctctctctggtttggtgcttctctccttcaaaaaccagtaacatccagccttctctctgcagagcccagcgggcccatttcaacaatcaacc

SEQ ID NO: 116 - DNA, 40 bp, Mus musculus
agaattgagaaagaatagagacctgcagttgaggccagca

SEQ ID NO: 117 - DNA, 40 bp, Mus musculus
agaattgagaaaaaatagaaatagcaactaggagggagct

SEQ ID NO: 118 - DNA, 40 bp, Mus musculus
aggaatatgagggaccagtctcagcagctaggagggagct

SEQ ID NO: 119 - DNA, 40 bp, Mus musculus
gtgaggtaccagtcctagcagctatggggcagctgggtat

SEQ ID NO: 120 - DNA, 40 bp, Mus musculus
gtgaggtaccagtcctagcagctgggctggactgagttga

SEQ ID NO: 121 - DNA, 40 bp, Mus musculus
atccaagctaggctgcctgagctgggctgggctgagctga

SEQ ID NO: 122 - DNA, 40 bp, Mus musculus
gtgtgagctgggctaggctgagctgagctggaatgagctg

SEQ ID NO: 123 - DNA, 40 bp, Mus musculus
gtgtgaactgggctaggctgatatactgatttgctaggac

SEQ ID NO: 124 - DNA, 40 bp, Mus musculus
cagcaaaggagaaaaggagaatatactgatttgctaggac

SEQ ID NO: 125 - DNA, 32 bp, Mus musculus
gcaagagggaactactatggttacgggtatgc

SEQ ID NO: 126 - DNA, 11 bp, Mus musculus
ctcaggtccta

SEQ ID NO: 127 - DNA, 37 bp, Mus musculus
caagatctcccccgtattactacggtagtagctactg

SEQ ID NO: 128 - DNA, 10 bp, Mus musculus
ctcaggtcct

SEQ ID NO: 129 - DNA, 16 bp, Mus musculus
gcaagagagggggact

SEQ ID NO: 130 - DNA, 11 bp, Mus musculus
ctcagtgaaca

SEQ ID NO: 131 - DNA, 32 bp, Mus musculus
aagacagggctatgatggttactacgtctggt

SEQ ID NO: 132 - DNA, 23 bp, Mus musculus
tgcaaggaaaggggtactctatg

SEQ ID NO: 133 - DNA, 33 bp, Mus musculus
gcaagagaggggtctatgattacgacggggttt

SEQ ID NO: 134 - DNA, 11 bp, Mus musculus
tgcaggtccta

SEQ ID NO: 135 - DNA, 21 bp, Mus musculus
gcaagagggattacccggggt

SEQ ID NO: 136 - DNA, 27 bp, Mus musculus
aatgcatggcatgatccgtcccacttt

SEQ ID NO: 137 - DNA, 30 bp, Mus musculus
aagactcgggctcgggctacggaggtatgt

SEQ ID NO: 138 - DNA, 35 bp, Mus musculus
aagacatgggaattactacggtagtagcctctatg

SEQ ID NO: 139 - DNA, 34 bp, Mus musculus
tactgttatatttattactacggcagggactact

SEQ ID NO: 140 - DNA, 32 bp, Mus musculus
ctgtgcagggggcttactacggcagtcctttg

SEQ ID NO: 141 - DNA, 21 bp, Mus musculus
gcaaaatggggagaatttgct

SEQ ID NO: 142 - DNA, 21 bp, Mus musculus
gcaagatggggggaatttcct

SEQ ID NO: 143 - DNA, 28 bp, Mus musculus
aagacataggactacggggctccctatg

SEQ ID NO: 144 - DNA, 33 bp, Mus musculus
gtgagacactactatgattacgggggttatgct

SEQ ID NO: 145 - DNA, 28 bp, Mus musculus
caagactagtgctcgggctacgtgctat

SEQ ID NO: 146 - DNA, 28 bp, Mus musculus
aagacatgatttattactaccttttgct

SEQ ID NO: 147 - DNA, 32 bp, Mus musculus
aagagattcctataggtacgctccagggggcc

SEQ ID NO: 148 - DNA, 294 bp, Mus musculus
caggtccaactgcagcagcctggggctgaactggtgaagcctggggcttcagtgaagctgtcctgcaaggcttctggctacaccttcaccagctactggatgcactgggtgaagcagaggcctggacaaggccttgagtggattggagagattaatcctagcaacggtcgtactaactacaatgagaagttcaagagcaaggccacactgactgtagacaaatcctccagcacagcctacatgcaactcagcagcccgacatctgaggactctgcggtctattactgtgcaaga

SEQ ID NO: 149 - DNA, 271 bp, Mus musculus
ggctgagctggtgaggcctggggtttcagtgaagctgtcctgcaaggcttctggctacacattcaccagttactgggtccactggattaagcggaggcctgaccaaggccttgagaggattggagagattaatccttacactggtgatactaactacaatgagaagttcaagaacaaggccacactgactgtagacaaatcctccagcacagcctacatgcaactcaacggcctggcatctgcggactctgcggtctattactgtgcgaga

SEQ ID NO: 150 - DNA, 271 bp, Mus musculus
ggctgagctggtgaggcctggggtttcagtgaaactgtcctgcaaggcttctggctacacattcaccagctactggatgcactggattaagcagaggcctgagcaaggccttgagaggattggagagattaatcctagcactggtggtgctaactacaatgagaagttcaagagcaaggccacactgactgtagacaaatcctccagcacagcctacatgcaactcagcagcctgacatctgaggactctgcggtctattactgtgcaaga

SEQ ID NO: 151 - DNA, 294 bp, Mus musculus
caggtccagctgcagcaatctggacctgagctggtgaagcctggggcttcagtgaagatatcctgcaaggcttctggctataccttcacaagctactatatacactgggtgaagcagaggcctggacagggccttgagtggattggatatatttatcctagagatggtagtactaattacaatgagaagttcaagggcaaggccacactgactgcagacacatcctccagcacagcctacatgcagctcagcagcctgacatctgaggactctgcagtctatttctgtgcaaga

SEQ ID NO: 152 - DNA, 271 bp, Mus musculus
ggctgagctggtgaggcctgggtcctcagtgaagatttcctgcaaggcttctggctatacattcagtagctactggatgaactgggtgaagcagaggcctggacagggtcttgagtggattggacagatttatcctggagatggtgatactaactacaatggaaagttcaggggtaaagccacactgactgcagacaaatcctccagcacagcctacatgcagctcagcagcctaacatctgaggactctgcggtctatttctgtgcaaga

SEQ ID NO: 153 - DNA, 271 bp, Mus musculus; identical to SEQ ID NO: 152

SEQ ID NO: 154 - DNA, 294 bp, Mus musculus
gaggtccagctgcaacaatctggacctgagctggtgaagcctggggcttcagtgaagatatcctgtaaggcttctggatacacgttcactgactactacatgaactgggtgaagcagagccatggaaagagccttgagtggattggagatattaatcctaacaatggtggtactagctacaaccagaagttcaagggcaaggccacattgactgtagacaagtcctccagcacagcctacatggagctccgcagcctgacatctgaggactctgcagtctattactgtgcaaga

SEQ ID NO: 155 - DNA, 396 bp, Mus musculus
cggccagtgaattgtaatacgactcactatagggcgaattgggccctctagatgcatgctcgagcggccgccagtgtgatggatatctgcagaattcggcttgaggtggagctgaaggagtcaggacctgagctggtgaagcctggggcttcagtgaagatgtcctgtaaggcttctggatacacattcactgactactacatgaagtgggtgaagcagagtcatggaaagagccttgagtggattggagatattaatcctaacaatggtggtactagctacaaccagaagttcaagggcaaggccacattgactgtagacaaatcctccagtacagcctacatgcagctcaacagcctgacatctgaggactctgcagtctattactgtgcaaga

SEQ ID NO: 156 - DNA, 296 bp, Mus musculus
gaagtgaagctggtggagtctgggggagacttagtgaagcctggagggtccctgaaactctcctgtgcagcctctggattcactttcagtagctatggcatgtcttgggttcgccagactccagacaagaggctggagtgggtcgcaaccattagtagtggtggtagttacacctactatccagacagtgtgaaggggcgattcaccatctccagagacaatgccaagaacaccctgtacctgcaaatgagcagtctgaagtctgaggacacagccatgtattactgtgcaagaca

SEQ ID NO: 157 - DNA, 274 bp, Mus musculus
ggggagacttagtgaagcctggagggtccctgaaactctcctgtgcagcctctggattcactttcagtagctatggcatgtcttgggttcgccagactccagacaagaggctggagtgggtcgcaaccattagtagtggtggtagttacacctactatccagacagtgtgaaggggcgattcaccatctccagagacaatgccaagaacaccctgtacctgcaaatgagcagtctgaagtctgaggacacagccatgtattactgtgcaagaca

SEQ ID NO: 158 - DNA, 293 bp, Mus musculus
gaagtgcagctggtggagtctgggggaggcttagtgaagcctggagggtccctgaaactctcctgtgcagcctctggattcactttcagtgactattacatgtattgggttcgccagactccggaaaagaggctggagtgggtcgcaaccattagtgatggtggtagttacacctactatccagacagtgtgaaggggcgattcaccatctccagagacaatgccaagaacaacctgtacctgcaaatgagcagtctgaagtctgaggacacagccatgtattactgtgcaag

SEQ ID NO: 159 - DNA, 272 bp, Mus musculus
gggggaggcttagtgaagcctggagggtccctgaaactctcctgtgcagcctctggattcactttcagtgactattacatgtattgggttcgccagactccggaaaagaggctggagtgggtcgcaatcattagtgatggtggtagtcacacctactatccagacagtgtgaaggggcgattcaccatctccagagacaatgccaagaacaacctgtacctgcaaatgagcagtctgaagtctgaggacacagccatgtattactgtgcaag

SEQ ID NO: 160 - DNA, 264 bp, Mus musculus
ggcttagtgaagcctggagggtccctgaaactctcctgtgcagcctctggattcactttcagtagctatgccatgtcttgggttcgccagactccagagaagaggctggagtgggtcgcatccattagtagtggtggtagcacctactatccagacagtgtgaagggccgattcaccatctccagagataatgccaggaacatcctgtacctgcaaatgagcagtctgaggtctgaggacacggccatgtattactgtgcaaga

SEQ ID NO: 161 - DNA, 278 bp, Mus musculus
tggagtctgggggaggcttagtgaagcctggagggtccctgaaactctcctgtgcagcctctggattcactttcagtagctatgccatgtcttgggttcgccagactccagagaagaggctggagtgggtcgcatccattagtagtggtggtagcacctactatccagacagtgtgaagggccgattcaccatctccagagataatgccaggaacatcctgtacctgcaaatgagcagtctgaggtctgaggacacggccatgtattactgtgcaaga

SEQ ID NO: 162 - DNA, 295 bp, Mus musculus
gaggtgcagctggtggagtctgggggaggcttagtgcagcctggagggtccctgaaactctcctgtgcagcctctggattcactttcagtagctatggcatgtcttgggttcgccagactccagacaagaggctggagttggtcgcaaccattaatagtaatggtggtagcacctattatccagacagtgtgaagggccgattcaccatctccagagacaatgccaagaacaccctgtacctgcaaatgagcagtctgaagtctgaggacacagccatgtattactgtgcaagag

SEQ ID NO: 163 - DNA, 276 bp, Mus musculus
ctgggggaggcttagtgcagcctggagggtccctgaaactctcctgtgcagcctctggattcactttcagtagctatggcatgtcttgggttcgccagactccagacaagaggctggagttggtcgcaaccattaatagtaatggtggtagcacctattatccagacagtgtgaagggccgattcaccatctccagagacaatgccaagaacaccctgtacctgcaaatgagcagtctgaagtctgaggacacagccatgtattactgtgcaagag

SEQ ID NO: 164 - DNA, 294 bp, Mus musculus; identical to SEQ ID NO: 108

SEQ ID NO: 165 - DNA, 278 bp, Mus musculus
agtctggggcagagcttgtgaggtcaggggcctcagtcaagttgtcctgcacagcttctggcttcaacattaaagactactatgtacactgggtgaagcagaggcctgcacagggcctggagtggattggatggattgatcctgagaatggtgatactgaatatgccccgaggttccagggcaaggccactatgactgcagacacatcctccaacacagcctacctgcagctcagcagcctgacatctgaggacactgccgtcttttactgtaatgca

SEQ ID NO: 166 - DNA, 296 bp, Mus musculus
gaggtgaagcttctcgagtctggaggtggcctggtgcagcctggaggatccctgaatctctcctgtgcagcctcaggattcgattttagtagatactggatgagttgggctcggcaggctccagggaaagggcaggaatggattggagaaattaatccaggaagcagtacgataaactatacgccatctctaaaggataaattcatcatctccagagacaacgccaaaaatacgctgtacctgcaaatgagcaaagtgagatctgaggacacagccctttattactgtgcaagact

SEQ ID NO: 167 - DNA, 273 bp, Mus musculus
aggtggcctggtgcagcctggaggatccctgaatctctcctgtgcagcctcaggattcgattttagtagatactggatgagttgggctcggcaggctccagggaaagggcaggaatggattggagaaattaatccaggaagcagtacgataaactatacgccatctctaaaggataaattcatcatctccagagacaacgccaaaaatacgctgtacctgcaaatgagcaaagtgagatctgaggacacagccctttattactgtgcaagact

SEQ ID NO: 168 - DNA, 1360 bp, Mus musculus
tctggacctctccgaaaccaggcaccgcaaatggtaagccagaggcagccacagctgtggctgctgctcttaaagcttgtaaactgtttctgcttaagagggactgagtcttcagtcattgctttagggggagaaagagacatttgtgtgtcttttgagtaccgttgtctgggtcactcacatttaactttccttgaaaaactagtaaaagaaaaatgttgcctgttaaccaataatcatagagctcatggtattttgaggaaatcttagaaaacgtgtatacaattgtctggaattatttcagttaagtgtattagttgaggtactgatgctgtctctacttcagttatacatgtgggtttgaattttgaatctattctggctcttcttaagcagaaaatttagataaaatggatacctcagtggtttttaatggtgggtttaatatagaaggaatttaaattggaagctaatttagaatcagtaaggagggacccaggctaagaaggcaatcctgggattctggaagaaaagatgtttttagtttttatagaaaacactactacattcttgatctacaactcaatgtggtttaatgaatttgaagttgccagtaaatgtacttcctggttgttaaagaatggtatcaaaggacagtgcttagatccaaggtgagtgtgagaggacaggggctggggtatggatacgcagaaggaaggccacagctgtacagaattgagaaagaatagagacctgcagttgaggccagcaggtcggctggactaactctccagccacagtaatgacccagacagagaaggccagactcataaagcttgctgagcaaaattaagggaacaaggttgagagccctagtaagcgaggctctaaaaagcatggctgagctgagatgggtgggcttctctgagcgcttctaaaatgcgctaaactgaggtgattactctgaggtaagcaaagctgggcttgagccaaaatgaagtagactgtaatgaactggaatgagctgggccgctaagctaaactaggctggcttaaccgagatgagccaaactggaatgaacttcattaatctaggttgaatagagctaaactctactgcctacactggactgttctgagctgagatgagctggggtgagctcagctatgctacgctgtgttggggtgagctgatctgaaatgagctactctggagtagctgagatggggtgagatggggtgagctgagctgggctgagctggactgagctgagctaggggggtgagctgagctgggtgagctgagctgagctggggtgagctgagctgagctgagctgagctggggtgagctgagctga

SEQ ID NO: 169 - DNA, 835 bp, Mus musculus
gctgctcttaaagcttgtaaactgtttctgcttaagagggactgagtcttcagtcattgctttagggggagaaagagacatttgtgtgtcttttgagtaccgttgtctgggtcactcacatttaactttccttgaaaaactagtaaaagaaaaatgttgcctgttaaccaataatcatagagctcatggtactttgaggaaatcttagaaagcgtgtatacaattgtctggaattatttcagttaagtgtattagttgaggtactgatgctgtctctacttcagttatacatgtgggtttgaattttgaatctattctggctcttcttaagcagaaaatttagataaaatggatacctcagtggtttttaatggtgggtttaatatagaaggaatttaaattggaagctaatttagaatcagtaaggagggacccaggctaagaaggcaatcctgggattctggaagaaaagatgtttttagtttttatagaaaacactactacattcttgatctacaactcaatgtggtttaatgaatttgaagttgcccgtaaatgtacttcctggttgttaaagaatggtatcaagggacagtatttagatccgaggtgagtgtgggaggacaggggctggggtatggatacgcagaaggaaggccacagctgtacagaattgagaaagaatagagacctgcagttgaggccagcaggtcggctggactaactctccagccacagtaatgacccagacagagaaagccagactcataaagcttgctgagcaaaattaagggaacaaggttgagagccctagtaagcgaggctctaaaaagca

SEQ ID NO: 170 - DNA, 935 bp, Mus musculus
cagctgtggctgctgctcttaaagcttgtaaactgtttctgcttaagagggactgagtcttcggtcattgctttagggggagaaagagacatttgtgtgtcttttgagtaccgttgtctgggtcactcacatttaactttccttgaaaaactagtaaaagaaaaatgttgcctgttaaccaataatcatagagctcatggtattttgaggaaatcttagaaaacgtgtatacaattgtctggaattatttcagttaagtgtattagttgaggtactgatgctgtctctacttcagttatacatgtgggtttgaattttgaatctattctggctcttcttaagcagaaaatttagataaaatggatacctcagtggtttttaatggtgggtttaatatagaaggaatttaaattggaagctaatttagaatcaataaggagggacccaggctaagaaggcaatcctgggattctggaagaaaagatgtttttagtttttatagaaaacactactacattcttgatctacaactcaatgtggtttaatgaatttgaagttgccagtaaatatacttcctggttgttaaagaatggtatcaaaggacagtgcttagatccaaggtgagtgtgagaggacaggggctggggtatggatacgcagaaggaaggccacagctgtacagaattgagaaagaatagagacctgcagttgaggccagcaggtcggctggactaactctccagccacagtaatgacccagacagagaaggccagactcataaagcttgctgaacaaaattaagggaacaaggttgagagccctagtaagcgaggctctaaaaagcacggctgagttgaggtgggtgggcttctctgagcgcttctaaaatgcgctaaactgagtgattactctgaggtaaacaaagctgggcttg

SEQ ID NO: 171 - DNA, 554 bp, Mus musculus
gtggctgctgctcttaaagcttgtaaactgtttctgcttaagagggactgagtcttcagtcattgctttagggggagaaagagacatttgtgtgtcttttgagtaccgttgtctgggtcactcacatttaactttccttgaaaaactagtaaaagaaaaatgttgcctgttaaccaataatcatagagctcatggtattttgaggaaatcttagaaaacgtgtatacaattgtctggaattatttcagttaagtgtattagttgaggtactgatgctgtctctacttcagttatacatgtgggtttgaattttgaatctattctggctcttcttaagcagaaaatttagataaaatggatacctcagtggtttttaatggtgggtttaatatagaaggaatttaaattggaagctaatttagaatcagtaaggagggacccaggctaagaaggcaatcctgggattctggaagaaaagatgtttttagtttttatagaaaacactactacattcttgatctacaactcaatgtggtttaatgaatttgaagttg

SEQ ID NO: 172 - DNA, 648 bp, Mus musculus
cagctgtggctgctgctcttaaagcttgtaaactgtttctgcttaagagggactgagtcttcagtcattgctttagggggagaaagagacatttgtgtgtcttttgagtaccgttgtctgggtcactcacatttaactttccttgaaaaactagtaaaagaaaaatgttgcctgttaaccaataatcatagagctcatggtactttgaggaaatcttagaaagcgtgtatacaattgtctggaattatttcagttaagtgtattagttgaggtactgatgctgtctctacttcagttatacatgtgggtttgaattttgaatctattctggctcttcttaagcagaaaatttagataaaatggatacctcagtggtttttaatggtgggtttaatatagaaggaatttaaattggaagctaatttagaatcagtaaggagggacccaggctaagaaggcaatcctgggattctggaagaaaagatgtttttagtttttatagaaaacactactacattcttgatctacaactcaatgtggtttaatgaatttgaagttgccagtaaatgtacttcctggttgttaaagaatggtatcaaaggacagtgcttagatccgaggtgagtgtgagaggacaggggctggggt

SEQ ID NO: 173 - DNA, 838 bp, Mus musculus
cagctgtggctgctgctcttaaagcttgtaaactgtttctgcttaagagggactgagtcttcagtcattgctttagggggagaaagagacatttgtgtgtcttttgagtaccgttgtctgggtcactcacatttaactttccttgaaaaactagtaaaagaaaaatgttgcctgttaaccaataatcatagagctcatggtactttgaggaaatcttagaaagcgtgtatacaattgtctggaattatttcagttaagtgtattagttgaggtactgatgctgtctctacttcagttatacacgtgggtttgaattttgaatctattctggctcttcttaagcagaaaatttagataaaatggatacctcagtggtttttaatggtgggtttaatatagaaggaatttaaattggaagctaatttagaatcagtaaggagggacccaggctaagaaggcaatcctgggattctggaagaaaagatgtttttagtttttatagaaaacactactacattcttgatctacaactcaatgtggtttaatgaatttgaagttgccagtaaatgtacttcctggttgttaaagaatggtatcaaaggacagtgcttagatccgaggtgagtgtgagaggacaggggctggggtatggatacgcagaaggaaggccacaactgtacagaattgagaaagaatagagacctgcagttgaggccagcaggtcggctggactaactctccagccacagtaatgacccagacagggaaagccagactcataaagattgctgagcaaaattaagggaacaaggttgagagccctagtaagcgaggctct

SEQ ID NO: 174 - DNA, 1143 bp, Mus musculus
gctcttaaagcttgtaaactgtttctgcttaagagggactgagtcttcagtcattgctttagggggagaaagagacatttgtgtgtcttttgagtaccgttgtctgggtcactcacatttaactttccttaaaaaactagtaaaagaaaaatgttgcctgttaaccaataatcatagagctcatggtactttgaggaaatcttagaaagcgtgtatccaattgtctggaattatttcagttaagtgtattagttgaggtactgatgctgtctctacttcagttatacatgtgggtttgaattttgaatctattctggctcttcttaagcagaaaatttagataaaatggatacctcagtggtttttaatggtgggtttaatatagaaggaatttaaattggaagctaatttagaatcagtaaggagggacccaggctaagaaggcaatcctgggattctggaagaaaagacatttttagtttttatagaaaataccattacattcttgatctacaactcaatgtggtttaaagaatttgaagttgccagtaaatgtacttcctggttgttaaagaatggtatcaaaggacagtgcttagatccgaggtgagtgtgagaggacaggggctggggtatggatacgcagaaggaaggccacagctatacagaattgagaaagaatagagacctgcagttgaggccagcaggtcggctggactaactctccagccacagtaatgacccagacagagaaagccagactcataaagcttgctgagcaaaattaagggaacaaggttgagagccctactaagcgagactctaaaaaacacagctgagctgagatgggtgggcttctctgagtgcttctaaaatgcgctaaactgaggtgattactctgaggtaagcaaagctgggcttgagccaagatgaagtagactgtaatgaactggaatgagctgggccgctaagctaaactaggctggcttaaccgagatgagccaaagaggaatgaacttcattaatctgggttgaatggagctaaactctactgcctacactggactgttttgatctgagatgacctggggtgagctcagctatgctacgtgtgttggggtgagctgatctgaaatgagatac

SEQ ID NO: 175 - DNA, 563 bp, Mus musculus
gctgctcttaaagcttgtaaactgtttctgcttaagagggactgagtcttcagtcattgctttagggggagaaagagacatttgtgtgtcttttgagtaccgttgtctgggtcactcacatttaactttccttgaaaaactagtaaaagaaaaatgttgcctgttaaccaatactcatagagctcatggtattttgaggaaatcttagaaaacgtgtatacaattgtctggaattatttcagttaagtgtattagttgaggtactgatgctgtctctacttcagttatgcatgtgggtttgaattttgaatctattctggctcttcttaagcagaaaatttagataaaatggatacctcagtggtttttaatggtgggtttaatatagaaggaatttaaattggaagctaatttagaatcagtaaggagggacccaggctaagaaggcaatcctgggattctggaagaaaagatgtttttagtttttatagaaaacactactacattcttgatctacaactcaatgtggtttaatgaatttgaagttgccagtaaatgtactt

SEQ ID NO: 176 - DNA, 763 bp, Mus musculus
gctgctcttaaagctggtaaactgtttctgcttaagagggactgagtcttcagtcattgctttagggggagaaagagacatttgtgtgtcttttgagtaccgttgtctgggtcactcacatttaactttccttgaaaaactagtaaaagaaaaatgttgcctgttaaccaataatcatagagctcatggtattttgaggaaatcttagaaaacgtgtatacaattgtctggaattatttcagttaagtgtattagttgaggtactgatgctgtctctacttcagttatacatgtgggtttgaattttgaatctattctggctcttcttaagcagaaaatttagataaaatggatacctcagtggtttttaatggtgggtttaatatagaaggaatttaaattggaagctaatttagaatcagtaaggagggacccaggctaagaaggcaatcctgggattctggaagaaaagatgtttttagtttttatagaaaacactactacattcttgatctacaactcaatgtggtttaatgaatttgaagttgccagtaaatgtacttcctggttgttaaagaatggtatcaaaggacagtgcttagatccaaggtgagtgtgagaggacaggggctggggtatggatacgcagaaggaaggccacagctgtacagaattgagaaagaatagagacctgcagttgaggccagcaggtcgtctggactaactctccagccacagtaatgacccagacagagaaggccag

SEQ ID NO: 177 - DNA, 1239 bp, Mus musculus
cgactgtggctgctgctcttaaagcttgtaaactgtttctgcttaagagggactgagtcttcagtcattgctttagggggagaaagagacatttgtgtgtcttttgagtaccgttgtctgggtcactcacatttaactttccttgaaaaactagtaaaagaaaaatgttgcctgttaaccaataatcatagagctcatggtattttgaggaaatcttagaaaacgtgtatacaattgtctggaattatttcagttaagtgtattagttgaggtactgatgctgtctctacttcagttatacatgtgggtttgaattttgaatctattctggctcttcttaagcagaaaatttagataaaatggatacctcagtggtttttaatggtgggtttaatatagaaggaatttaaattggaagctaatttagaatcagtaaggagggacccaggctaagaaggcaatcctgggattctggaagaaaagatgtttttagtttttatagaaaacactactacattcttgatctacaactcaatgtggtttaatgaatttgaagttgccagtaaatgtacttcctggttgttaaagaatggtatcaaaggacagtgcttagatccaaggtgagtgtgagaggacaggggctggggtatggatacgcagaaggaaggccacagctgtacagaattgagaaagaatagagacctgcagttgaggccagcaggtcggctggactaactctccagccacagtaatgacccagacagagaaggccagactcataaagcttgctgagcaaaattaagggaacaaggttgagagccctagtaagcgaggctctaaaaagcatggctgagctgagatgggtgggcttctctgagcgcttctaaaatgcgctaaactgaggtgattactctgaggtaagcaaagctgggcttgagccaaaatgaagtagactgtaatgaactggaatgagctgggccgctaagctaaactaggctggcttaaccgagatgagccaaactggaatgaacttcattaatctaggttaaatagagctaaactctactgcctaccctggactgttctgagctgagatgagctggggtgagctcagctatgctacgctgtgttggggtgagctgatctgaaatgagctactctggagtagctgagatggggtgagatggggtgagccgagctgggctgagatggactgagctgagctagggtgagctgagc

SEQ ID NO: 178 - DNA, 866 bp, Mus musculus
tcttaaagctcgtaaactgtttctgcttaagagggactgagtcttcagtcattgctttagggggagaaagagacatttgtgtgtcttttgagtaccgttgtctgggtcactcacatttaactttccttgaaaaactagtaaaagaaaaatgttgcctgttaaccaataatcatagagctcatggtattttgaggaaatcttagaaaacgtgtatacaattgtctggaattatttcagttaagtgtattagttgaggtactgatgctgtctctacttcagttatacatgtgggtttgaattttgaatctattctggctcttcttaagcagaaaatttagataaaatggatacctcagtggtttttaatggtgggtttaatatagaaggaatttaaattggaagctaatttagaatcagtaaggagggacccaggctaagaaggcaatcctgggattctggaagaaaagatgtttttagtttttatagaaaacactactacattcttgatctacaactcaatgtggtttaatgaatttgaagttgccagtaaatgtacttcctggttgttaaagaatggtatcaaaggacagtgtttagatccaaggtgagtgtgagaggacaggggctggggtatggatacgcagaaggaaggccacagctgtacagaattgagaaagaatagagacctgcagttgaggccagcaggtcggctggactaactctccagccacagtaatgacccagacagagaaggccagactcataaagcttgctgagcaaaattaagggaacaaggttgagagccctagtaagcgaggctctaaaaagcatggctgagctgagatgggtgggcttctctgagcgct

SEQ ID NO: 179 - DNA, 314 bp, Mus musculus
cgaaaccaggcaccgcaaatggtaagccagaggcagccacagctgtggctgctgctcttaaagcttgtaaactgtttctgcttaagagggactgagtcttcagtcattgctttagggggagaaagagacatttgtgtgtcttttgagtaccgttgtctgggtcactcacatttaactttccttgaaaaactagtaaaagaaaaatgttgcctgttaaccaataatcatagagctcatggtattttgaggaaatcttagaaaacgtgtatacaattgtctggaattatttcagttaagtgtattagttgaggtac

SEQ ID NO: 180 - DNA, 517 bp, Mus musculus
cagctgtggctgctgctcttaaagcttgtaaactgtttctgcttaagagggactgagtcttcagtcattgctttagggggagaaagagacatttgtgtgtcttttgagtaccgttgtctgggtcactcacatttaactttccttgaaaaactagtaaaagaaaaatgttgcctgttaaccaataatcatagaactcatggtattttgaggaaatcttagaaaacgtgtatgcaattgtctggaattatttcagttaagtgtattagttgaggtactgatgctgtctctacttcagttatacatgtgggtttgaattttgaatctattctggctcttcttaagcagaaaatttagataaaatggatacctcagtggtttttaatggtgggtttaatatagaaggaatttaaattggaagctaatttagaatcagtaaggagggacccaggctaagaaggcaatcctgggattctggaagaaaagatgtttttagtttttatagaaaacactagtacat

SEQ ID NO: 181 - DNA, 475 bp, Mus musculus
gaaaccaggcaccgcaaatggtaagccagaggcagccacagctgtggctgctgctcttaaagcttgtaaactgtttctgcttaagagggactgagtcttcagtcattgctttagggggagaaagagacatttgtgtgtcttttgagtaccgttgtctgggtcactcacatttaactttccttgaaaaactagtaaaagaaaaatgttgcctgttaaccaataatcatagagctcatggtattttgaggaaatcttagaaaacgtgtatacaattgtctggaattatttcagttaagtgtattagttgaggtactgatgctgtctctacttcagttatacatgtgggtttgaattttgaatctattctggctcttcttaagcagaaaatttagataaaatggatacctcagtggtttttaatggtgggtttaatatagaaggaatttaaattggaagctaatttagaatcagtaat

SEQ ID NO: 182 - DNA, 130 bp, Mus musculus
agagggactgagtcttcagtcattgctttagggggagaaagagacatttgtgtgtcttttgagtaccgttgtctgggtcactcacatttaactttccttgaaaaactagtaaaagaaaaatgttgcctat

SEQ ID NO: 183 - DNA, 888 bp, Mus musculus
gggactgagtcttcagtcattgctttagggggagaaagagacatttgtgtgtcttttgagtaccgttgtctgggtcactcacatttaactttccttgaaaaactagtaaaagaaaaatgttgcctgttaaccaataatcatagagctcatggtattttgaggaaatcttagaaaacgtgtatacaattgtctggaattatttcagttaagtgtattagttgagatactgatgctgtctctacttcagttatacatgtgggtttgaattttgaatctattctggctcttcttaagcagaaaatttagataaaatggatacctcagtggtttttaatggtgggtttaatatagaaggaatttaaattggaagctaatttagaatcagtaaggagggacccaggctaagaaggcaatcctgggattctggaagaaaagatgtttttagtttttatagaaaacactactacattcttgatctacaactcaatgtgatttaatgaatttgaagttgccagtaaatgtacttcctggttgttaaagaatggtatcaaaggacagtgcttagatccaagatgagtgtgagaggacaggggctggggtatggatccgcagaaggaaggccacaactgtacagaattgagaaagaatagagacttgcagttgaggccagcaggtcggctggactaactctccagccacagtaatgacccagacagagaaggccagactcataaagtttgctgagcaaagtaagcgaggctctaaaaggctgagctgagatgggtgggcttctctgagcgcttctaaaatgcgctaaactgaggtgattactctgaggtaaacaaagctgggcttgagccaaaatgaagtagactgtaatgaactgga

SEQ ID NO: 184 - DNA, 445 bp, Mus musculus
gggactgagtcttcagtcattgctttagggggagaaagagacatttgtgtgtcttttgagtaccgttgtctgggtcactcacatttaactttccttgaaaaactagtaaaagaaaaatgttgcctgttaaccaataatcatagagctcatggtattttgaggaaatcttagaaaacgtgtatacaattgtctggaattatttcagttaagtgtattagttgaggtactgatgctgtctctacttcagttatacatgtgggtttgaattttgaatctattctggctcttcttaagcagaaaatttagataaaatggatacctcagtggtttttaatggtgggtttaatatagaaggaatttaaattggaaactaatttagaatcagtaaggaaggacccaggctaagaaggcaatcctgggattctggaagaaaagatgtttttag

SEQ ID NO: 185 - DNA, 698 bp, Mus musculus
gagggactgagtcttcagtcattgctttagggggagaaagagacatttgtgtgtcttttgagtaccgttgtctgggtcactcacatttaactttccttgaaaaactagtaaaagaaaaatgttgcctgttaaccaataatcatagagctcatggtattttgaggaaatcttagaaaacgtgtatacaattgtctggaattatttcagttaagtgtattagttgaggtactgatgctgtctctacttcagttatacatgtgggtttgaattttgaatctattctggctcttcttaagcagaaaatttagataaaatggatacctcagtggtttttaatggtgggtttaatatagaaggaatttacccaggctaagaaggcaatcctgggattctggaagaaaagatgtttttagtttttatagaaaacactactacattcttgatctacaactcaatgtggtttaatgaatttgaagttgccagtaaatgtacttcctggttgttaaagaatggtatcaaaggacagtgcttagatctaaggtgagtgtgagaggacaggggctggggtatggatacgcagaaggaaggccacagctgtacagaattgagaaagaatagagacctgcagttgaggccagcaggtcggctggactaactctccagccacagtaatgacccagacagagaaggccagactc

SEQ ID NO: 186 - DNA, 592 bp, Mus musculus
tgagtccgtgtctgggtcactcacatttaactttccttgaaaaactagtaaaagaaaaatgttgcctgttaaccaataatcatagagctcatggtattttgaggaaatcttagaaaacgtgtatacaattgtctggaattatttcagttaagtgtattagttgaggtactgatgctgtctctacttcagttatacatgtgggtttgaattttgaatctattctggctcttcttaagcagaaaatttagataaaatggatacctcagtggtttttaatggtgggtttaatatagaaggaatttaaattggaagctaatttagaatcagtaaggagggacccaggctaagaaggcaatcctgggattctggaagaaaagatgtttttagtttttatagaaaacactaatacattcttgatctacaactcaatgtggtttaatgaatttgaagttgccagtaaatgtacttcctggttgttaaagaatggtatcaaaggacagtacttagatccaaggtgagtgtgagaggacaggggctggggtatggatacgcagaaggaaggccacagctgtacagaattgagaaaaaatag

SEQ ID NO: 187 - DNA, 992 bp, Mus musculus
tgagtccgtgtctgggtcactcacatttaactttccttgaaaaactagtaaaagaaaaatgttgcctgttaaccaataatcatagagctcatggtattttgaggaaatcttagaaaacgtgtatacaattgtctggaattatttcagttaagtgtattagttgaggtactgatgctgtctctacttcagttatacatgtgggtttgaattttgaatctattctggctcttcttaagcagaaaatttagataaaatggatacctcagtggtttttaatggtgggtttaatatagaaggaatttaaattggaagctaatttagaatcagtaaggagggacccaggctaagaaggcaatcctgggattctggaagaaaagatgtttttagtttttatagaaaacactaatacattcttgatctacaactcaatgtggtttaatgaatttgaagttgccagtaaatgtacttcctggttgttaaagaatggtatcaaaggacagtacttagatccaaggtgagtgtgagaggacaggggctggggtatggatacgcagaaggaaggccacagctgtacagaattgagaaaaaatagtgtgctagactgagctgtactggatgatctggtgtagggtgatctggactcaactgggctggctgatgggatgccccaggttgaactaggctcagataagttaggctgagtagggcctggttgagatggttcgggatgagctgggaaaagatggactgggaccatgaactgggctgagctgggttgggagaccatgaattgagctgaactgagtgcagctgggataaactgggttgagctaagaatagactacctgaattgtgccaaactgggctgggatcaattggaaattatcaggatttagatgagccggactaaactatgctgagctggactggttggatgtgttgaactggcctgctgctgggctggcatagctgagttgaacttaaatgaggaa

SEQ ID NO: 188 - DNA, 357 bp, Mus musculus
tgagttgtactggatgatctggtgtagggtgatctggactcaactgggctggctgatgggatgccccaggttgaactaggctcagataagttaggctgagtagggcctggttgagatggttcgggatgagctgggaaaagatggactgggaccatgaactgggctgagttgggttgggagaccatgaattgagctgaactgagtgcagctgggataaactgggttgagctaagaatagactacctgaattgtgccaaactgggctgggatcaattggaaattatcaggatttagatgagccggactaaactatgctgagctggactggttggatgtgttgaactggcctactgctgg

SEQ ID NO: 189 - DNA, 80 bp, Mus musculus
ctgggcagctctggggagctagggtgggtgggatgtggggaccaggctgggcagctctgaggagctggggtaggtggggt

SEQ ID NO: 190 - DNA, 57 bp, Mus musculus
gggagctagggtgggtgggatgtggggaccaggctgggcagctctgaggagctgggg

SEQ ID NO: 191 - DNA, 160 bp, Mus musculus
gagggaccagtctcagcagctaggagggagctggggcaggtgggagtgtgagggaccagacctagcagctgtgggtgacttgcagatgttggaaatgtgaggtaccagtcctagcagctatggggcagctgggtatagttggaatatgggggaccagatc

SEQ ID NO: 192 - DNA, 102 bp, Mus musculus
aaatagcaactaggagggagctggggcaggtgggagtgtgagggaccagacctagctgtgggtgacttgcagatgttggaaatgtgaggtaccagtcctagc

SEQ ID NO: 193 - DNA, 80 bp, Mus musculus
aaacaggcagcacaggtgcaggtgacctaatgagaggtgagtagtacagagccatggtacctccaaaagctcagaaaccc

SEQ ID NO: 194 - DNA, 43 bp, Mus musculus
aatgagaggtgagtagtacagagtcatggtacctccaaaagct

SEQ ID NO: 195 - DNA, 80 bp, Mus musculus
gagctaccctagggactactggacaaatcagagcagctaaagggtaggatctgggaatgagaggaatgtggggggccagt

SEQ ID NO: 196 - DNA, 39 bp, Mus musculus
taccctagggactactggacaaatcagagtagttaaaga

SEQ ID NO: 197 - DNA, 240 bp, Mus musculus
ggtaggagtgtgaggccaaggcctaaaagctattggggagctggggatagtaggaagtgggcaaccagcccaagcagctatggggtagctggtgatggtaggaatggggaaaataatgtttgcagctacagaggagctagagacagtagtacaaaatcctagcagttatgggggaggtggatatggtaggaaagagagtaaatgctcccagcagctgtgggacagatggggaaagtagga

SEQ ID NO: 198 - DNA, 208 bp, Mus musculus
agaccgtgtgaggccaaggcctaaaagttattggggagctggggatagtaggaagtgggcaaccagcccaagcagatatggggtaactggtgatggtaggaatggggaaaataatgtttgcagctacagaggagctagagacagtagtacaaaatcctagcagttatgggggaggtggatatggtaggaaagagagtaaatgctccca

SEQ ID NO: 199 - DNA, 240 bp, Mus musculus; identical to SEQ ID NO: 197

SEQ ID NO: 200 - DNA, 143 bp, Mus musculus
ggaaagtagtgatgtgagaatctatatttaccaggtataggggaactgggtcatgtagaaatatgagaggacaaaacctgcagccatggggaaactctgaagtataaaaaagtttaatgaggagctaaagtagagctgaaaaa

SEQ ID NO: 201 - DNA, 80 bp, Mus musculus
actggctgggctggaatttgctgggctgtgctgagctgggataaactagagtaagtagactggccaaaataggctgggat

SEQ ID NO: 202 - DNA, 32 bp, Mus musculus
gggataaagtagagtaagtagactggccaaaa

SEQ ID NO: 203 - DNA, 160 bp, Mus musculus
aactgagctagactggtctgaggcgggctaatctgggatgaggtggactgagctgggctaagctaaattaagctgagatgagctaggctagacttactgagctaggctggaataggctaggctaagctaggctgcctgagctaagcttggctgagatgaa

SEQ ID NO: 204 - DNA, 83 bp, Mus musculus
ctagactggtctgaggcggactaatcttactgagctaggctggaataggctaggctaaactaggctgcctgagctaagcttgg

SEQ ID NO: 205 - DNA, 320 bp, Mus musculus
gaataaactggactggactagctaaactagattggcatggtctgtgctgacctggactgggctagggttggatgggctcaataactgggctaatccaagctaggctgcctgagctgggctgggctgagctgagctaggctggaataggctgggctgggctgggctggtgtgagctgggctaggctgagctgagctggaatgagctgggatgggctgaactaggctggaataggctgggctggctggtgtgagctgggctaggctgagctgagctggaatgagctgggattggctagaataggctgggctggactagtgtt

SEQ ID NO: 206 - DNA, 243 bp, Mus musculus
cagctgaccgagtgggctagggttggatgggctcaataactgggctaatccaagctaggttgcctgagctgggctaggctgagctgagctaggctggaataggctgggctaggctcatgtgagctgggctaggctgagctgagctggaatgagctgggatgagctgatctaggctggaataggctgggctggctggtgtgagctgaactaggctgagctgagctggaatgaactgggattggc

SEQ ID NO: 207 - DNA, 75 bp, Mus musculus
agctgggctggactgagttgagctaggctggaataggctgggctgggctgggctggtgtgaactgggctaggctg

SEQ ID NO: 208 - DNA, 320 bp, Mus musculus
aggctggactgggctggtgtgtgagctaggttaggctgggctgagctggaatgagctgggttgaactgagcaaggctggatggaataggctgggctgggctggtgtgagctgggctaggctgagctgagctgggatgagctgagctaggctagaataggctgggctggactagtgttagctgggttaggctgggctgagctggaatgagctgggatgagcagagctaggctggaataggctgggctgggctggtgtgagctgggttaggctgagctgagctgagctggaatgagctgggatgagctgagctaggctggaa

SEQ ID NO: 209 - DNA, 240 bp, Mus musculus
ctgagcaaggctggatggaataaactgggctgggctggtgtgagctgggctaggctgagctgaactgggatgagctgagctaggctagaataggctgggctggactagtgttagctgggttaggctgggctgagtttgaatgagctgggatgagtagagttaggctggaataggctgggctgggctggtgtgaggtggattaggctgcgctgagctgaggtggaatgagctgggatgagg

SEQ ID NO: 210 - DNA, 1598 bp, Mus musculus
tgatccgagctgaaatgagctgagataagattagctaggctggaataggctgggctgggctggtgtgtgctaggttggtctgagctgagctggaatgagctgggatgggctgagctaggctggaataggttgggctgggctggtgtgagctgggttaggctgagctgagctggaatgagctaggatgagctgagctaggctggaataggctgggtttggctggtgtgagctgagttaggctgatccgagctgaaatgagctgagataagattagctaggctggaataggctgggctgggctggtgtgtgctaggttggtctgagctgagctggaatgagctgggatgggctgagctaggctggaataggttgggctgggctggtgtgagctgggttaggctgagttgagctggaatgagattagatgagctgagctaggctggagtaggctgggctgggctggtgcgagctgggttaggctgagctgagctggaatgagatggaataggctgggctggctggtgtgagctgggctaggctgagctgagctaaactaggctgaaatgggctgagcagagctggacaaagctaggctacactgcactgtctggctaggctgtactggaatgagctgagctgagctgggctaagctgggatggactaggataaactaagctgggatgagacaggctggactgcaggaggaagactggaagggctggctgagctagactaggctgggctgagctggaatgagctgggttgagctgaactagtataaacttggctaggctacaatggattgagctgagctagacttagggtggaatgggctgaacaaggctgagcttacctagaccgggcagacctagatagagttgcactgaggtaggttagacagggttgtctgagctgagctgacctaggcaagctgtgctgtctgagctggcctaagatggacttagttgaggtgaagtgagaactaggctggaatgggctttctgaactgggctgaaatggcctgagctgggctggactggactggaatgaattagtctgggctaggctgagttagtctgggctaggctgagttagtctggactaggctgagttagtctgggctaggctgagttagtctgggctaggctgagttagtctgggctaggctgagttagtctaggctggaccaaattaggctggatgggctaaactgagctgaactaggatgggatgggatgggatgggatgggatgggatgaactgactgggctggactcagttgaccttgctcgtctgagctggtctagatggtctagttgggctggccaggatagtcagaactaggctggaattaggcgaaacttggcttggctggttacaatgagctaacataaattcagctggctgaaccaaacttgacagtgagctagcctggggtgaattagcatgactggacttattcacagttctagcctgagctttgctggattgttaaactcactgctgaggtaactgaaaagactttggatgaaatgtgaaccaactcttgttttatggtccagattgtggcctctggtttgctttgtgtgaatggagc

SEQ ID NO: 211 - DNA, 51 bp, Mus musculus
tggatgggctaaactgagctgaactaggatgggatgggatgggatgggatg

SEQ ID NO: 212 - DNA, 336 bp, Mus musculus
atgggatgggctgagctaggctggaataggttgggctgggctggtgtgagctgggttaggctgagctgagctggaatgagctaggatgagctgaactaggctggaataggctgggtttggctggtgtgagctgagttaggctgatccaagcatgaaatgagctgagataagattagctaggctggaataggctgggctggactggtgtgtgctaggttggtctgaactgagctgggatgggctgagctaggctggaataggttgggctgggctggtgtgagctgggttaggctgagttgagctggaatgagattagatgagctgagctaggctgga

SEQ ID NO: 213 - DNA, 325 bp, Mus musculus
ggatgagatgggatgaactgactgggctgggctcagttgaccttgctcgtttgagctggtctagatggtctagttgggctggccagaatagtcagagctaggctggaattaggcgaaacttggcttggctggttacaatgagctaacataaattcagctggctgaaccaaacttgacagtgagctagcctggggtgaattagcatgactggacttattcacagttctagcctgactttgctggattgttaaactcatgctgaggtaactgaaaagactttggatgaaatgtgaaccaactctgttttatggtccagattgaggcc

SEQ ID NO: 214 - DNA, 402 bp, Mus musculus
tgggctggtgcgagctgggttaggctgagctgaactggaatgagatgaaataggctgggctggctggtgtgagctgggctaggctgagctgaactaaactaggctgaaatggcctgagcagagctggacaaagctaggctacactgcactgtctggctaggctgtactggaatgagctgagctgagctgggctaagctgggatggactaggataaactaagctgggatgagacaggctggactgcaggaggaagactggaagggctggctgagctagactagactgggctgagctggaatgagctgggttgagttgaactagtataaacttggctaggctacaatggattgagctgagctagacttagggtggaatgggctgaacaaggctgagcttaccta

SEQ ID NO: 215 - DNA, 246 bp, Mus musculus
agttgaccttgctcgtctgagctggtctagatggtctagttgggctggccaggatagtcagaactaggctggaattaggcaaacttggcttggctggttacaatgagctaacataaattcagctggctgaaccaaacttgacagtgagctagcctggggtgaattagcatgactggacttattcacagttctagcctgagctttgctggattgttaaactcactgctgaggtaactgaaaagactt

SEQ ID NO: 216 - DNA, 46 bp, Mus musculus
tgggatgggatgggatgggatgggatgaactgactgggctggactc

SEQ ID NO: 217 - DNA, 73 bp, Mus musculus
ctaaaataaattcagctggctgaaccaaacttgacagtgagctagcctggggtgaattagcatgactggactt

SEQ ID NO: 218 - DNA, 394 bp, Mus musculus
ccgggcagacctagatagagttgcactgaggtaggttagacagggttgtctgagctgagctgacctaggcaaactgtgctgtctgagttggcctaagtatggacttatttgaggtgaagtgagaaccaggctggaatgggctttctgaactggactgaaatggcctgatctgggctggactggactagaatgaattagtctgggctagactgagttagtctgggctaggctgagttagtctagactaggctgagttagtctgggctaggctgagttagtctgggctaggctgagttagtctgggctaggctgagtgagtctaggctggaccaaattaggctggatggactaaactgagctgaactaagatgggatgggatgggatgggatggga

SEQ ID NO: 219 - DNA, 220 bp, Mus musculus
taggctggagtaggctgggctgggctggtgcgagctgggttaggctgaaatgagctggaatgagatggaataggctgggctggccaggatagtcagaactaggctggaattaggcaaacttggcttgactggttacaatgagttaacataaattcagctggctgaaccaaacttgacagtgagctagcctggggtgaattagcatgactggacttattca

SEQ ID NO: 220 - DNA, 133 bp, Mus musculus
taggctggaataggctgggctgggctggtgtgtgctaggttggtctgagctgagctggaatgagctgggatgggctgagctaggctggaataggttgggctgggctggtgtgagctgggttaggctgagctga

SEQ ID NO: 221 - DNA, 1280 bp, Mus musculus
gtgaaaacacaacaaaatggccttctccaccccacacacaaccccctgccccgccagtgtaacttccaagccagctcttccccaaccactcctaccccgtgctggctgatcttcagtctcaggttggccacccctgcccagacccaccagttctggtcctcctaaccctgggactgagaacatagctctatccactgcccaaacaggagtggggcttagaaatctggagcgctagactgctcaggggtgtagtcatgtttacaaacgcacagtatgtgcaaagccctgctagagtcatttggctagatccttgtgaaagactacctgcaggtcatgttcaaagtctatacagccagaactgttggtcagctccgactgcgggtacacaatgcagcagctatgtgatactgggctaggttctcctgtataaagaagagaaaggcatggtcctttttcctgaaattgtctcgagatgggcagtgtgaagactactatctcatgcatgttttatgttccagagtctgcgagaaatcccaccatctacccactgacactcccaccagctctgtcaagtgacccagtgataatcggctgcctgattcacgattacttcccttccggcacgatgaatgtgacctggggaaagagtgggaaggatataaccaccgtaaacttcccacctgccctggcctctgggggacggtacaccatgagcagccagttgaccctgccagctgtcgagtgcccagaaggagaatccgtgaaatgttccgtgcaacatgactctaaccccgtccaagaattggatgtgaattgctctggtaaagaacgttagggggtcagctaggggtgggataagtcctaccttatctagatccatatatccctctgaggcacaccctcacagggaccctcagaaacctcccatggggttgggggaagggaagcgtaaacaggccagaaggagctgaggcctcagaacatccagaaaaggggacagcaaaggagaaaaggagaatatactgatttgctaggacttctctgttacaggtcctactcctcctcctcctattactattccttcctgccagcccagcctgtcactgcagcggccagctcttgaggacctgctcctgggttcagatgccagcatcacatgtactctgaatggcctgagaaatcctgagggagctgtcttcacctgggagccctccactgggaaggatgcagtgcagaagaaagctgtgcagaattcctgcggctgctacagtgtgtccagc

SEQ ID NO: 222 - DNA, 1122 bp, Mus musculus
gatcttcagtctcaggttggccacccctgcccagacccaccagttctggtcctcctaaccctgggactgagaacatagctctatccactgcccaaacaggagtggggctcagaaatctggagcgctagactgctcaggggtgtagtcatgtttacaaacgcacagtatgtgcaaagccctgctggagtcatttggctagatccttgtgaaagactacctgcaggtcatgttcaaagtctatacagccagaactgttggtcagctccgactgcaggtacacgatgcagcagctgtgtgatactgggctaggttctcctgtataaagaagagaaaggcatggtcctttttcctgaaattgtctcgagatgggcagtgtgaagactactatctcatgcatgttttatgttccagagtctgcgagaaatcccaccatctacccactgacactcccacgagctctgtcaagtgacccagtgataatcggctgcctgattcacgattacttcccttccggcacgatgaatgtgacctggggaaagagtgggaaggatataaccaccgtaaacttcccacctgccctggcctctgggggggggtacaccatgagcagccagttgaccctgccagctgtcgagtgcccagaaggagaatccgtgaaatgttccgtgcaacatgactctaacgccgtccaagaattggatgtgaagtgctctggtaaagaacgttagggggtcagctgggggtgggataagttctaccttatctagatccatatatccctctgaggcacaccctcacagggaccctcagaaacctcccatgggggtgggggaagggaagcgtaaacaggccataaggaactgaggcctcagaacatccagaaaaggggacagcaaaggagaaaaggagaatatactgatttgctaggacttctctgttacaggtcctcctcctccttgtcctccttgtcctccttcctgccatcccagcctgtcactgcagcggccagctcttgaggacctgctcctgggttcagatgccagcctcacatgtactctgaatggcctgagaaatcctgagggagctgtcttcacctgggagccctccactgggaaggatgca

SEQ ID NO: 223 - DNA, 1168 bp, Mus musculus
caaccccctgccccgccagggtaacttccaagccagctcttccccaaccactcctaccccgtgctggctgatcttcagtctcaggttggccacccctgcccagacccaccagttctggtcctcctaaccctgggactgagaacatagctctatccactgcccaaacaggagtggggcttagaaatctggagcgctagactgctcaggggtgtagtcatgtttacaaacgcacagtatgtgcaaagccctgctagagtcatttggctagatccttgtgaaagactacctgcaggtcatgttcaaagtctatacagccagaactgttggtcagctccgactgcgggtacacaatgcagcagctatgtgatactgggctaggttctcctgtataaagaagagaaaggcatggtcctttttcctgaaattgtctcgagatgggcagtgtgaagactactatctcatgcatgttttatgttccagagtctgcgagaaatcccaccatctacccactgacactcccaccagctctgtcaagtgacccagtgataatcggctgcctgattcacgattacttcccttccggcacgatgaatgtgacctggggaaagagtgggaaggatataaccaccgtaaacttcccacctgccctggcctctgggggacggtacaccatgagcagccagttgaccctgccagctgtcgagtgcccagaaggagaatccgtgaaatgttccgtgcaacatgactctaaccccgtccaagaattggatgtgaattgctctggtaaagaacgttagggggtcagctaggggtgggataagtcctaccttatctagatccatatatccctctgaggcacaccctcacagggaccctcagaaacctcccatggggttgggggaagggaagcgtaaacaggccagaaggagctgaggcctcagaacatccagaaaaggggacagcaaaggagaaaaggagaatatactgatttgctaggacttctctgttacaggtcctactcctcctcctcctattactattccttcctgccagcccagcctgtcactgcagcggccagctcttgaggacctgctcctgggttcagatgccagcatcacatgtactctgaatggcctgagaaatcctgagggagctgtcttcacctggg

SEQ ID NO: 224 - DNA, 1100 bp, Mus musculus
gatcttcagtctcaggttggccacccctgcccagacccaccagttctggtcctcctaaccctgggactgagaacatagctctatccactgcccaaacaggagtggggcttagaaatctggagcgctagactgctcaggggtgtagtcatgtttacaaacgcacagtatgtgcagagccctgctagagtcatttggctagatccttgtgaaagactacctgcaggtcatgttcaaagtctatacagccagaactgttggtcagctccgactgcgggtacacaatgcagcagctatgtgatactgggctaggttctcctgtataaagaagagaaaggcatggtcctttttcctgaaattgtctcgagatgggcagtgtgaagactactatctcatgcatgttttatgttccagagtctgcgagaaatcccaccatctacccactgacactcccaccagctctgtcaagtgacccagtgataatcggctgcctgattcacgattacttcccttccggcacgatgaatgtgacctggggaaagagtgggaaggatataaccaccgtaaacttcccacctgccctggcctctgggggacggtacaccatgagcagccagttgaccctgccagctgtcgagtgcccagaaggagaatccgtgaaatgttccgtgcaacatgactctaaccccgtccaagaattggatgtgaattgctctggtaaagaacgttagggggtcacctaggggtgtgataagtcctaccttatctagatccatatatccctctgaggcacaccctcacagggaccctcagaaacctcccatggggttgggggaagggaagcgtaaacaggccagaaggagctgaggcctcagaacatccagaaaaggggacagcaaaggagaaaaggagaatatactgatttgctaggacttctctgttacaggtcctactcctcctcctcctattactattccttcctgccagcccagcctgtcactgcagcggccagctcttgaggacctgctcctgggttcagatgccagcatcacatgtactctgaatggcctgagaaatcctgagggagctgtcttcacctggga

SEQ ID NO: 225 - DNA, 1170 bp, Mus musculus
caaccccctgtcccgccagggtaacttccaagccagctcttccccaaccactcctaccccgcgctggctgatcttcagtctcaggttggccacccctgcccagacccaccagttctggtcctcctaaccctgggactgagaaccactgcccaaacaggagtggggctcagaaatctggagcgctagactgctcaggggtgtagtcatgtttacaaacgcacagtatgtgcaaagccctgctggagtcatttggctagatccttgtgaaagactacctgcaggtcatgttcaaagtctatacagccagaactgttggtcagctccgactgcaggtacacgatgcagcagctgtgtgatactgggctaggttctcctgtataaagaagagaaaggcatggtcctttttcctgaaattgtctcgagatgggcagtgtgaagactactatctcatgcatgttttatgttccagagtctgcgagaaatcccaccatctacccactgacactcccacgagctctgtcaagtgacccagtgataatcggctgcctgattcacgattacttcccttccggcacgatgaatgtgacctggggaaagagtgggaaggatataaccaccgtaaacttcccacctgccctggcctctgggggagggtacaccatgagcagccagttgaccctgccagctgtcgagtgcccagaaggagaatccgtgaaatgttccgtgcaacatgactctaacgccgtccaagaattggatgtgaagtgctctggtaaagaacgttagggggtcagctgggggtgggataagttctaccttatctagatccatatatccctctgaggcacaccctcacagggaccctcagaaacctcccatgggggtggggggagggaagcgtaaacaggccataaggaactgaggcctcagaacatccagaaaaggggacagcaaaggagaaaaggagaatatactgatttgctaggacttctctgttacaggtcctcctcctccttgtcctccttgtcctccttcctgccatcccagcctgtcactgcagcggccagctcttgaggacctgctcctgggttcagatgccagcctcacatgtactctgaatggcctgagaaatcctgagggagctgtcttcacctgggagccctccactgg

SEQ ID NO: 226 - DNA, 916 bp, Mus musculus
gggtcatttggctagatccttgtgaaagactacctgcaggtcatgttcaaagtctatacagccagaactgttggtcagctccgactgcaggtacacgatgcagcagctgtgtgatactgggctaggttctcctgtataaagaagagaaaggcatggtcctttttcctgaaattgtctcgagatgggcagtgtgaagactactatctcatgcatgttttatgttccagagtctgcgagaaatcccaccatctacccactgacactcccacgagctctgtcaagtgacccagtgataatcggctgcctgattcacgattacttcccttccggcacgatgaatgtgacctggggaaagagtgggaaggatataaccaccgtaaacttcccacctgccctggcctctgggggagggtacaccatgagcagccagttgaccctgccagctgtcgagtgcccagaaggagaatccgtgaaatgttccgtgcaacatgactctaacgccgtccaagaattggatgtgaagtgctctggtaaagaacgttagggggtcagctgggggtgggataagttctaccttatctagatccatatatccccctgaggcacaccctcacagggaccctcagaaacctcccatgggggtgggggaagggaagcgtaaacaggccataaggaactgaggcctcagaacatccagaaaaggggacagcaaaggagaaaaggagaatatactgatttgctaggacttctctgttacaggtcctcctcctccttgtcctccttgtcctccttcctgccatcccagcctgtcactgcagcggccagctcttgaggacctgctcctgggttcagatgccagcctcacatgtactctgaatggcctgagaaatcctgagggagctgtcttcacctggga

SEQ ID NO: 227 - DNA, 474 bp, Mus musculus
acaccatgagcagccagttgaccctgccagctgtcgagtgcccagaaggagaatccgtgaaatgttccgtgcgacatgactctaacgccgtccaagaattggatgtgaagtgctctggtaaagaacgttagggggtcagctgggggtgggataagttctaccttatctagatccacatatccctctgaggcacaccctcacagggaccctcagaaacctcccatgggggtgggggaagggaagcgtaaacaggccataaggaactgaggcctcagaacatccagaaaaggggacagcaaaggagaaaaggagaatatactgatttgctaggacttctctgttacaggtcctcctcctccttgtcctccttgtcctccttcctgccatcccagcctgtcactgcagcggccagctcttgaggacctgctcctgggttcagatgccagcctcacatgtactctgaatggcctgaga

SEQ ID NO: 228 - DNA, 825 bp, Mus musculus
tgcagcagctatgtgatactgggctaggttctcctgtataaagaagagaaaggcatggtcctttttcctgaaattgtctcgagatgggcagtgtgaagactactatctcatgcatgttttatgttccagagtctgcgagaaatcccaccatctacccactgacactcccaccagctctgtcaagtgacccagtgataatcggctgcctgattcacgattacttcccttccggcacgatgaatgtgacctggggaaagagtgggaaggatataaccaccgtaaacttcccacctgccctggcctctgggggacggtacaccatgagcagccagttgaccctgccagctgtcgagtgcccagaaggagaatccgtgaaatgttccgtgcaacatgactctaaccccgtccaagaattggatgtgaattgctctggtaaagaacgttagggggtcagctagggatgggataagtcctaccttatctagatccatatatccctctgaggcacaccctcacagggaccctcagaaacctcccatggggttgggggaagggaagcgtaaacaggccagaaggggctgaggcctcagaacatccagaaaaggggacagcaaaggagaaaaggagaatatactgatttgctaggacttctctgttacaggtcctactcctcctcctcctattactattccttcctgccagcccagcctgtcactgcagcggccagctcttgaggacctgctcctgggttcagatgccagcatcacatgtactctgaatggcctgagaaatcctgagggagctgtcttcacctgggagccctcc

SEQ ID NO: 229 - DNA, 1129 bp, Mus musculus
tcctaccccgtgctggctgatcttcagtctcaggttggccacccctgcccagacccaccagttctggtcctcctaaccctgggactgagaacatagctctatccactgcccaaacaggagtggggcttagaaatctggagcgctagactgctcagaggtgtagtcatgtttacaaacgcacagtatgtgcaaagccctgctagagtcatttggctagatccttgtgaaagactacctgcaggtcatgttcaaagtctatacagccagaactgttggtcagctccgactgcgggtacacaatgcagcagctatgtgatactgggctaggttctcctgtataaagaagagaaaggcatggtcctttttcctgaaattgtctcgagatgggcagtgtgaagactactatctcatgcatgttttatgttccagagtctgcgagaaatcccaccatctacccactgacactcccaccagctctgtcaagtgacccagtgataatcggctgcctgattcacgattacttcccttccggcacgatgaatgtgacctggggaaagagtgggaaggatataaccaccgtaaacttcccacctgccctggcctctgggggacggtacaccatgagcagccagttgaccctgccagctgtcgagtgcccagaaggagaatccgtgaaatgttccgtgcaacatgactctaaccccgtccaagaattggatgtgaattgctctggtaaagaacgttagggggtcagctagggtgggataagtcctaccttatctagatccatatatccctctgaggcacaccctcacagggaccctcagaaacctcccatggggttgggggaagggaagcgtaaacaggccagaaggagctgaggcctcagaacatccagaaaaggggacagcaaaggagaaaaggagaatatactgatttgctaggacttctctgttacaggtcctactcctcctcctcctattactattccttcctgccagcccagcctgtcactgcagcggccagctcttgaggacctgctcctgggttcagatgccagcatcacatgtactctgaatggcctgagaaatcctgagggagctgtcttcacctgggagccctccactgg

SEQ ID NO: 230 - DNA, 515 bp, Mus musculus
tgggggacggtacaccatgagcagccagttgaccctgccagctgtcgagtgcccagaaggagaatccgtgaaatgttccgtgcaacatgactctaaccccgtccaagaattggatgtgaattgctctggtaaagaacgttagggggtcagctaggggtgggataagtcctaccttatctagatccatatatccctctgaggcacaccctcacagggaccctcagaaacctcccatggggttgggggaagggaagcgtaaacaggccagaaggagctgaggcctcagaacatccagaaaaggggacagcaaaggagaaaaggagaatatactgatttgctaggacttctctgttacaggtcctactcctcctcctcctattactattccctcctgccagcccagcctgtcactgcagcggccagctcttgaggacctgctcctgggttcagatgccagcatcacatgtactctgaatggcctgagaaatcctgagggagctgtcttcacctgggag

SEQ ID NO: 231 - DNA, 236 bp, Mus musculus
tccagaaaaggggacagcaaaggagaaaaggagaatatactgatttgctaggacttctctgttacaggtcctactcctcctcctcctattactattccttcctgccagcccagcctgtcactgcagcggccagctcttgaggacctgctcctgggttcagatgccagcatcacatgtactctgaatggcctgagaaatcctgagggagctgtcttcacctgggagccctccactgg

SEQ ID NO: 232 - DNA, 661 bp, Mus musculus
accagctctgtcaagtgacccagtgataatcggctgcctgattcacgattacttcccttccggcacgatgaatgtgacctggggaaagagtgggaaggatataaccaccgtaaacttcccacctgccctggcctctgggggacggtacaccatgagcagccagttgaccctgccagctgtcgagtgcccagaaggagaatccgtgaaatgttccgtgcaacatgactctaaccccgtccaagaattggatgtgaattgttctggtaaagaacgttagggggtcagctaggggtgggataagtcctaccttatctagatccatatatccctctgaggcacaccctcacagggaccctcagaaacctcccatggggttgggggaagggaagcgtaaacaggccagaaggagctgaggcctcagaacatccagaaaaggggacagcaaaggagaaaaggagaatatactgatttgctaggacttctctgttacaggtcctactcctcctcctcctattactattccttcctgccagcccagcctgtcactgcagcggccagctcttgaggacctgctcctgggttcagatgccagcatcacatgtactctgaatggcctgagaaatcctgagggagctgtcttcacctgggagccctccactgg

SEQ ID NO: 233 - DNA, 316 bp, Mus musculus
aatgttccgtgcaacatgactctaaccccgtccaagaattggatgtgaattgctctggtaaagaacgttagggggtcagctaggggtgggataagtcctaccttatctagatccatatatccctctgagaggacttctctgttacaggtcctactcctcctcctcctattactattccttcctgccagcccagcctgtcactgcagcggccagctcttgaggacctgctcctgggttcagatgccagcatcacatgtactctgaatggcctgagaaatcctgagggagctgtcttcacctgggagccctccactgg

SEQ ID NO: 234 - DNA, 251 bp, Mus musculus
tgaggcctcagaacatccagaaaaggggacagcaaaggagaaaaggagaatatactgatttgctaggacttctctgttacaggtcctactcctcctcctcctattactattccttcctgccagcccagcctgtcactgcagcggccagctcttgaggacctgctcctgggttcagatgccagcatcacatgtactctgaatggcctgagaaatcctgagggagctgtcttcacctgggagccctccactgg

SEQ ID NO: 235 - DNA, 948 bp, Mus musculus
aacactagactgctcagggtgtagtcatgtttacaaacgcacagtatgtgcaaagccctgttagagtcatttggctagatccttgtgaaagactacctgcaggtcatgttcaaagtctatacagccagaactgttggtcagcttcgactgcgggtacacaatgcagcagctatgtgatactgggctaggttctcctgtataaagaagagaaaggcatggtcctttttcctggaattgtctcgagatgggcagtgtgaggactactatctcatgcatgttttatgttccagagtctccgagaaatcccaccatctacccactgacactcccaccagctctgtcaagtgacccagtgataatcggcagcctgattcacgcttacttcccttccggcacgatgactgtgacctggggaaagagtgggaaggatataaccaccgtaaacttcccacctgccctggcctctgggggacggtacaccatgagcagccagttgaccctgccagctgtcgagtgcccagaaggagaatccgtgaaatgttccgtgcaacatgactctaaccccgtccaagaattggatgtgaattgctctggtaaagaacgttagggggtcagataggggtgggataagtcctaccttatctagatccatatatccctctgaggcacaccctcacagggaccctcagaaacctcccatggggttgggggaagggaagcgtaaacaggccagaaggagctgaggcctcagaacatccagaaaaggggacagcaaaggagaaaaggagaatatactgatttgctaggacttctctgttacaggtcctactcctcctcctcctattactattccttcctgccagcccagcctgtcactgcagcggccagctcttgaggacctgctcctgggttcagatgccagcatcacatgtactctgaatggcctga

SEQ ID NO: 236 - DNA, 616 bp, Mus musculus
cccaaccactcctaccccgtgctggctgatcttcagtctcaggttggccacccctgcccagacccaccagttctggtcctcctaaccctgggactgagaacatagctctatccactgcccaaacaggacctctgggggacggtacaccatgagcagccagttgaccctgccagctgtcgagtgcccagaaggagactccgtgaaatgttccgtgcaacatgactctaaccccgtccaagaattggatgtgaattgctctggtaaagaacgttagggggtcaactaggggtgggataagtcctaccttatctagatccatatatccctctgaggcacaccctcacagggaccctcagaaacctcccatggggttgggggaagggaagcgtaaacaggccagaaggagctgaggcctcagaacatccagaaaaggggacagcaaaggagaaaaggagaatatactgatttgctaggacttctctgttacaggtcctactcctcctcctcctattactattccttcctgccagcccagcctgtcactgcagcggccagctcttgaggacctgctcctgggttcagatgccagcatcacatgtactctgaatggcctgag

SEQ ID NO: 237 - DNA, 322 bp, Mus musculus
taagtcctaccttatctagatccatatatccctctgaggcacaccctcacagggaccctcagaaacctcccatggggttgggggaagggaagcgtaaacaggccagaaggagctgaggcctcagaacatccagaaaaggggacagcaaaggagaaaaggagaatatactgatttgctaggacttctctgttacaggtcctactcctcctcctcctattactattccttcctgccagcccagcctgtcactgcagcggccagctcttgaggacctgctcctgggttcagatgccagcatcacatgtactctgaatggcctgag

SEQ ID NO: 238 - DNA, 258 bp, Mus musculus
aacctcccatggggttgggggaagggaagcgtaaacaggccagaaggagctgaggcctcagaacatccagaaaaggggacagcaaaggagaaaaggagaatatactgatttgctaggacttctctgttacaggtcctactcctcctcctcctattactattccttcctgccagcccagcctgtcactgcagcggccagctcttgaggacctgctcctgggttcagatgccagcatcacatgtactctgaatggcctga

SEQ ID NO: 239 - DNA, 150 bp, Mus musculus
atatactgatttgctaggacttctctgttacaggtcctactcctcctcctcctattactattccttcctgccagcccagcctgtcactgcagcggccagctctttgaggacctgctcctgggttcagatgccagcatcacatgtactctg

SEQ ID NO: 240 - protein, 45 aa, Mus musculus
GAELVRPGVSVKLSCKASGYTFTSYWMHWIKQRPEQGLERIGEIN

SEQ ID NO: 241 - protein, 9 aa, Mus musculus
PSTGGANYN

SEQ ID NO: 242 - protein, 45 aa, Mus musculus
GAELVRPGVSVKLSCKASGYTFTSYWVHWIKRRPDQGLERIGEIN

SEQ ID NO: 243 - protein, 9 aa, Mus musculus
PYTGDTNYN

SEQ ID NO: 244 - protein, 46 aa, Mus musculus
SGAELVRPGSSVKISCKASGYTFSSYWMNWVKQRPGQGLEWIGQIY

SEQ ID NO: 245 - protein, 9 aa, Mus musculus
PGDGDTNYN

SEQ ID NO: 246 - protein, 52 aa, Mus musculus
EVELKESGPELVKPGASVKMSCKASGYTFTDYYMKWVKQSHGKSLEWIGDIN

SEQ ID NO: 247 - protein, 9 aa, Mus musculus
PNNGGTSYN

SEQ ID NO: 248 - protein, 44 aa, Mus musculus
GDLVKPGGSLKLSCAASGFTFSSYGMSWVRQTPDKRLEWVATIS

SEQ ID NO: 249 - protein, 9 aa, Mus musculus
SGGSYTYYP

SEQ ID NO: 250 - protein, 45 aa, Mus musculus
GGGLVKPGGSLKLSCAASGFTFSDYYMYWVRQTPEKRLEWVAIIS

SEQ ID NO: 251 - protein, 9 aa, Mus musculus
DGGSHTYYP

SEQ ID NO: 252 - protein, 43 aa, Mus musculus
GLVKPGGSLKLSCAASGFTFSSYAMSWVRQTPEKRLEWVASIS

SEQ ID NO: 253 - protein, 3 aa, Mus musculus
SGG

SEQ ID NO: 254 - protein, 5 aa, Mus musculus
STYYP

SEQ ID NO: 255 - protein, 45 aa, Mus musculus
GGGLVQPGGSLKLSCAASGFTFSSYGMSWVRQTPDKRLELVATIN

SEQ ID NO: 256 - protein, 9 aa, Mus musculus
SNGGSTYYP

SEQ ID NO: 257 - protein, 47 aa, Mus musculus
ESGAELVRSGASVKLSCTASGFNIKDYYVHWVKQRPAQGLEWIGWID

SEQ ID NO: 258 - protein, 9 aa, Mus musculus
PENGDTEYA

SEQ ID NO: 259 - protein, 45 aa, Mus musculus
GGGLVQPGGSLNLSCAASGFDFSRYWMSWARQAPGKGQEWIGEIN

SEQ ID NO: 260 - protein, 9 aa, Mus musculus
PGSSTINYT

SEQ ID NO: 261 - protein, 44 aa, Mus musculus; identical to SEQ ID NO: 248

SEQ ID NO: 262 - protein, 9 aa, Mus musculus
SGGSYTYYP

SEQ ID NO: 263 - protein, 45 aa, Mus musculus
GSELVRPGASVKLSCKASGYTFTSYWMHWVKQRHGQGLEWIGNIY

SEQ ID NO: 264 - protein, 9 aa, Mus musculus
PGSGSTKYD

SEQ ID NO: 265 - protein, 45 aa, Mus musculus
GAELVKPGASVKLSCTASGFNIKDTYMHWVKQRPEQGLEWIGRID

SEQ ID NO: 266 - protein, 9 aa, Mus musculus
PANGNTKYD

SEQ ID NO: 267 - protein, 45 aa, Mus musculus
GAELARPGASVKMSCKASGYTFTSYAIHWVKQRPGQGLEWIGYIN

SEQ ID NO: 268 - protein, 9 aa, Mus musculus
PSSGYTNYN

SEQ ID NO: 269 - protein, 47 aa, Mus musculus
QSGAELARPGASVKMSCKASGYTFSAYAVHWVKQRPGQGLEWIGYIN

SEQ ID NO: 270 - protein, 9 aa, Mus musculus
PSSGYTNYN

SEQ ID NO: 271 - protein, 47 aa, Mus musculus
ESGGDLVKPGGSLKLSCAASGFTFSRYGMSWVRQTPDKRLEWVATIS

SEQ ID NO: 272 - protein, 9 aa, Mus musculus
SGGNYTYYP

SEQ ID NO: 273 - protein, 56 aa, Mus musculus
GGGLVQPKGSLKLSCAASGFTFNTYAMNWVRQAPGKGLEWVARIRSKSNDYSTYYA

SEQ ID NO: 274 - protein, 49 aa, Mus musculus
LVESGGGLVKPGGSLKLSCAASGFAFSSYDMSWVRQTPEKRLEWVATIS

SEQ ID NO: 275 - protein, 9 aa, Mus musculus
SGGSYTYYP

SEQ ID NO: 276 - protein, 50 aa, Mus musculus
QLVESGGGLVKPGGSLKLSCAASGFAFSSYDMSWVRQTPEKRLEWVAYIS

SEQ ID NO: 277 - protein, 9 aa, Mus musculus
SGGGSTYYP

SEQ ID NO: 278 - protein, 45 aa, Mus musculus; identical to SEQ ID NO: 255

SEQ ID NO: 279 - protein, 9 aa, Mus musculus
SNGGSTYYP

SEQ ID NO: 280 - protein, 45 aa, Mus musculus
EKFKSKATLTVDKSSSTAYMQLSSLTSEDSAVYYCARGNYYGYGY

SEQ ID NO: 281 - protein, 15 aa, Mus musculus
AMDYWGQGTSVTVSS

SEQ ID NO: 282 - protein, 45 aa, Mus musculus
EKFKNKATLTVDKSSSTAYMQLNGLASADSAVYYCARGNYYGYGY

SEQ ID NO: 283 - protein, 15 aa, Mus musculus
AMDFWGQGTSVTVSS

SEQ ID NO: 284 - protein, 63 aa, Mus musculus
GKFRGKATLTADKSSSTAYMQLSSLTSEDSAVYFCARSPPYYYGSSYCAMDYWGQGTSVTVSS

SEQ ID NO: 285 - protein, 41 aa, Mus musculus
QKFKGKATLTVDKSSSTAYMQLNSLTSEDSAVYYCAREGDY

SEQ ID NO: 286 - protein, 10 aa, Mus musculus
WGQGTSVTVS

SEQ ID NO: 287 - protein, 42 aa, Mus musculus
DSVKGRFTISRDNAKNTLYLQMSSLKSEDTAMYYCARQGYDG

SEQ ID NO: 288 - protein, 19 aa, Mus musculus
YYVWYFDVWGAGTTVTVSS

SEQ ID NO: 289 - protein, 39 aa, Mus musculus
DSVKGRFTISRDNAKNNLYLQMSSLKSEDTAMYYCARKG

SEQ ID NO: 290 - protein, 18 aa, Mus musculus
VLYAMDYWGQGTSVTVSS

SEQ ID NO: 291 - protein, 40 aa, Mus musculus
DSVKGRFTISRDNARNILYLQMSSLRSEDTAMYYCAREGS

SEQ ID NO: 292 - protein, 19 aa, Mus musculus
MITTGFAYWGQGTLVTVSA

SEQ ID NO: 293 - protein, 38 aa, Mus musculus
DSVKGRFTISRDNAKNTLYLQMSSLKSEDTAMYYCARG

SEQ ID NO: 294 - protein, 3 aa, Mus musculus
ITR

SEQ ID NO: 295 - protein, 10 aa, Mus musculus
GQGTSVTVSS

SEQ ID NO: 296 - protein, 43 aa, Mus musculus
PRFQGKATMTADTSSNTAYLQLSSLTSEDTAVFYCNAWHDPSH

SEQ ID NO: 297 - protein, 14 aa, Mus musculus
FDYWGQGTPLTVSS

SEQ ID NO: 298 - protein, 41 aa, Mus musculus
PSLKDKFIISRDNAKNTLYLQMSKVRSEDTALYYCARLGLG

SEQ ID NO: 299 - protein, 18 aa, Mus musculus
LRRYVMDYWGQGTSVTVS

SEQ ID NO: 300 - protein, 43 aa, Mus musculus
DSVKGRFTISRDNAKNTLYLQMSSLKSEDTAMYYCARHGNYYG

SEQ ID NO: 301 - protein, 19 aa, Mus musculus
SSLYAMDYWGQGTSVTVSS

SEQ ID NO: 302 - protein, 36 aa, Mus musculus
EKFKNKGTLTVDTSSSTAHMHLSSLTSEDSAVYYCY

SEQ ID NO: 303 - protein, 6 aa, Mus musculus
IYYYGR

SEQ ID NO: 304 - protein, 13 aa, Mus musculus
DYWGQGTTLTVSS

SEQ ID NO: 305 - protein, 43 aa, Mus musculus
PKFQGKATITADTSSNTAYLQLSSLTSEDTAVYYCAGGLLRQS

SEQ ID NO: 306 - protein, 14 aa, Mus musculus
FDYWGQGTTLTVSS

SEQ ID NO: 307 - protein, 43 aa, Mus musculus
QQFKDKATLTADKSSSTAYMQLSSLTSEDSAVYYCAKWGEFAY

SEQ ID NO: 308 - protein, 11 aa, Mus musculus
WGQGTLVTVSA

SEQ ID NO: 309 - protein, 43 aa, Mus musculus
QQFKDKATLTADKSSSTAYMQLTSLTSEDSAVYYCARWGEFPY

SEQ ID NO: 310 - protein, 11 aa, Mus musculus
WGQGTLVTVSA

SEQ ID NO: 311 - protein, 39 aa, Mus musculus
DSVKGRFTISRDNAKNTLYLQMSSLKSEDTAMYYCARHR

SEQ ID NO: 312 - protein, 19 aa, Mus musculus
TTGLPMDYWGQGTSVTVSS

SEQ ID NO: 313 - protein, 41 aa, Mus musculus
DSVKDRFTISRDDSQSMLYLQMNNLKTEDTAIYYCVRHYYD

SEQ ID NO: 314 - protein, 19 aa, Mus musculus
YGGYALDYWGQGTSVTVSS

SEQ ID NO: 315 - protein, 40 aa, Mus musculus
DSVKGRFTISRDNARNTLYLQMSSLRSEDTALYYCARLVL

SEQ ID NO: 316 - protein, 3 aa, Mus musculus
GLR

SEQ ID NO: 317 - protein, 15 aa, Mus musculus
AMDYWGQGTSVTVSS

SEQ ID NO: 318 - protein, 40 aa, Mus musculus
DTVKGRFTISRDNAKNTLYLQMSSLKSEDTAMYYCARHDL

SEQ ID NO: 319 - protein, 3 aa, Mus musculus
LLP

SEQ ID NO: 320 - protein, 14 aa, Mus musculus
FAYWGQGTLVTVSA

SEQ ID NO: 321 - protein, 38 aa, Mus musculus
DSVKGRFTISRDNAKNTLYLQMSSLKSEDTAMYYCARD

SEQ ID NO: 322 - protein, 17 aa, Mus musculus
SYRYAPGGQGTLVTVSA

SEQ ID NO: 323 - DNA, 40 bp, Mus musculus
gagccctagtaagcgaggctctaaaaagcatggctgagct

SEQ ID NO: 324 - DNA, 40 bp, Mus musculus
gcagctctgaggagctggggtaggtggggtataggaacca

SEQ ID NO: 325 - DNA, 40 bp, Mus musculus
gagccctagtaagcgaggctctggggagctagggtgggtg

SEQ ID NO: 326 - DNA, 40 bp, Mus musculus
gcagctctgaggagctgggggggtcatttggctagatcct

SEQ ID NO: 327 - DNA, 40 bp, Mus musculus
tgggaacaggctgggcagctctggggagctagggtgggtg

SEQ ID NO: 328 - DNA, 40 bp, Mus musculus
agttgccagtaaatgtacttcctggttgttaaagaatggt

SEQ ID NO: 329 - DNA, 40 bp, Mus musculus
tgagctggactgagctgagctagggtgagctgagctgggt

SEQ ID NO: 330 - DNA, 40 bp, Mus musculus
agttgccagtaaatgtacttggggtgaactgatctgaaat

SEQ ID NO: 331 - DNA, 40 bp, Mus musculus
tgagctggactgagctgagctgcagcagctatgtgatact

SEQ ID NO: 332 - DNA, 40 bp, Mus musculus
cagctatgctacgctgtgttggggtgagctgatctgaaat

SEQ ID NO: 333 - DNA, 40 bp, Mus musculus
ctccgactgcgggtacacaatgcagcagctatgtgatact

SEQ ID NO: 334 - DNA, 40 bp, Mus musculus
tatagaaaacactactacattcttgatctacaactcaatg

SEQ ID NO: 335 - DNA, 40 bp, Mus musculus
gaattagcatgactggacttattcacagttctagcctgag

SEQ ID NO: 336 - DNA, 40 bp, Mus musculus
gatccatatatccctctgaggcacaccctcacagggaccc

SEQ ID NO: 337 - DNA, 40 bp, Mus musculus
tatagaaaacactagtacatctaaaataaattcagctggc

SEQ ID NO: 338 - DNA, 40 bp, Mus musculus
gaattagcatgactggacttaatgttccgtgcaacatgac

SEQ ID NO: 339 - DNA, 40 bp, Mus musculus
gatccatatatccctctgagaggacttctctgttacaggt

SEQ ID NO: 340 - DNA, 40 bp, Mus musculus
gcttggctggttacaatgagctaacataaattcagctggc

SEQ ID NO: 341 - DNA, 40 bp, Mus musculus
cccagaaggagaatccgtgaaatgttccgtgcaacatgac

SEQ ID NO: 342 - DNA, 40 bp, Mus musculus
ggagaatatactgatttgctaggacttctctgttacaggt

SEQ ID NO: 343 - DNA, 40 bp, Mus musculus
agctagggtgagctgagctgggtgagctgagctaagctgg

SEQ ID NO: 344 - DNA, 40 bp, Mus musculus
tgaggtaactgaaaagactttggatgaaatgtgaaccaac

SEQ ID NO: 345 - DNA, 40 bp, Mus musculus
agctagggtgagctgagctgggatgggatgggatgggatg

SEQ ID NO: 346 - DNA, 40 bp, Mus musculus
tgaggtaactgaaaagactttgggggacggtacaccatga

SEQ ID NO: 347 - DNA, 40 bp, Mus musculus
agctgaactaggatgggatgggatgggatgggatgggatg

SEQ ID NO: 348 - DNA, 40 bp, Mus musculus
ttcccacctgccctggcctctgggggacggtacaccatga

SEQ ID NO: 349 - DNA, 40 bp, Mus musculus
aagaaaagatgtttttagtttttatagaaaacactactac

SEQ ID NO: 350 - DNA, 40 bp, Mus musculus
aaagagagtaaatgctcccagcagctgtgggacagatggg

SEQ ID NO: 351 - DNA, 40 bp, Mus musculus
gctaaagcagagctgaaacagatgctatggacaagttaaa

SEQ ID NO: 352 - DNA, 40 bp, Mus musculus
ctggtctgaggcgggctaatctgggatgaggtggactgag

SEQ ID NO: 353 - DNA, 40 bp, Mus musculus
gctgcctgagctaagcttggctgagatgaaccataatgag

SEQ ID NO: 354 - DNA, 40 bp, Mus musculus
tggaatgagctgggatgagctgagctaggctggaataggc

SEQ ID NO: 355 - DNA, 40 bp, Mus musculus
gctgggttaggctgagctgagctggaatgagctaggatga

SEQ ID NO: 356 - DNA, 40 bp, Mus musculus
aagaaaagatgtttttagagaccgtgtgaggccaaggcct

SEQ ID NO: 357 - DNA, 40 bp, Mus musculus
aaagagagtaaatgctcccaggaaagtagtgatgtgagaa

SEQ ID NO: 358 - DNA, 40 bp, Mus musculus
gctaaagtagagctgaaaaactagactggtctgaggcgga

SEQ ID NO: 359 - DNA, 40 bp, Mus musculus
ctggtctgaggcggactaatcttactgagctaggctggaa

SEQ ID NO: 360 - DNA, 40 bp, Mus musculus
gctgcctgagctaagcttggctgagcaaggctggatggaa

SEQ ID NO: 361 - DNA, 40 bp, Mus musculus
tggaatgagctgggatgaggtaggctggaataggctgggc

SEQ ID NO: 362 - DNA, 40 bp, Mus musculus
gctgggttaggctgagctgataagtcctaccttatctaga

SEQ ID NO: 363 - DNA, 40 bp, Mus musculus
tgggggacctgaggatggtaggagtgtgaggccaaggcct

SEQ ID NO: 364 - DNA, 40 bp, Mus musculus
aacaactatgaaaaacttaaggaaagtagtgatgtgagaa

SEQ ID NO: 365 - DNA, 40 bp, Mus musculus
actgggctgggctaactgagctagactggtctgaggcggg

SEQ ID NO: 366 - DNA, 40 bp, Mus musculus; identical to SEQ ID NO: 359

SEQ ID NO: 367 - DNA, 40 bp, Mus musculus; identical to SEQ ID NO: 360

SEQ ID NO: 368 - DNA, 40 bp, Mus musculus; identical to SEQ ID NO: 361

SEQ ID NO: 369 - DNA, 40 bp, Mus musculus; identical to SEQ ID NO: 362

SEQ ID NO: 370 - DNA, 40 bp, Mus musculus
gtagactgtaatgaactggaatgagctgggccgctaagct

SEQ ID NO: 371 - DNA, 40 bp, Mus musculus
ccatggtacctccaaaagctcagaaaccctggcagagcag

SEQ ID NO: 372 - DNA, 40 bp, Mus musculus
caaatcagagcagctaaagggtaggatctgggaatgagag

SEQ ID NO: 373 - DNA, 40 bp, Mus musculus
agtagactggccaaaataggctgggatggtctgtactggg

SEQ ID NO: 374 - DNA, 40 bp, Mus musculus
atgagatggaataggctgggctggctggtgtgagctgggc

SEQ ID NO: 375 - DNA, 40 bp, Mus musculus
agcatgactggacttattcacagttctagcctgagctttg

SEQ ID NO: 376 - DNA, 40 bp, Mus musculus
tatccactgcccaaacaggagtggggcttagaaatctgga

SEQ ID NO: 377 - DNA, 40 bp, Mus musculus
gtagactgtaatgaactggaaatgagaggtgagtagtaca

SEQ ID NO: 378 - DNA, 40 bp, Mus musculus
tcatggtacctccaaaagcttaccctagggactactggac

SEQ ID NO: 379 - DNA, 40 bp, Mus musculus
caaatcagagtagttaaagagggataaagtagagtaagta

SEQ ID NO: 380 - DNA, 40 bp, Mus musculus
agtagactggccaaaataggctggagtaggctgggctggg

SEQ ID NO: 381 - DNA, 40 bp, Mus musculus
atgagatggaataggctgggctggccaggatagtcagaac

SEQ ID NO: 382 - DNA, 40 bp, Mus musculus
agcatgactggacttattcacgccagtgtaacttccaagc

SEQ ID NO: 383 - DNA, 40 bp, Mus musculus
tatccactgcccaaacaggacctctgggggacggtacacc

SEQ ID NO: 384 - DNA, 40 bp, Mus musculus
agcacaggtgcaggtgacctaatgagaggtgagtagtaca

SEQ ID NO: 385 - DNA, 40 bp, Mus musculus
cagagtctgcacctcagagctaccctagggactactggac

SEQ ID NO: 386 - DNA, 40 bp, Mus musculus
ttgctgggctgtgctgagctgggataaactagagtaagta

SEQ ID NO: 387 - DNA, 40 bp, Mus musculus
attagatgagctgagctaggctggagtaggctgggctggg

SEQ ID NO: 388 - DNA, 40 bp, Mus musculus
gtctagatggtctagttgggctggccaggatagtcagaac

SEQ ID NO: 389 - DNA, 40 bp, Mus musculus
ccacacacaaccccctgccccgccagtgtaacttccaagc

SEQ ID NO: 390 - DNA, 40 bp, Mus musculus
aaacttcccacctgccctggcctctgggggacggtacacc

SEQ ID NO: 391 - DNA, 40 bp, Mus musculus
tttaatatagaaggaatttaaattggaagctaatttagaa

SEQ ID NO: 392 - DNA, 40 bp, Mus musculus
agacagagaaggccagactcataaagcttgctgagcaaaa

SEQ ID NO: 393 - DNA, 40 bp, Mus musculus
ttgaactggcctgctgctgggctggcatagctgagttgaa

SEQ ID NO: 394 - DNA, 40 bp, Mus musculus
tttaatatagaaggaatttacccaggctaagaaggcaatc

SEQ ID NO: 395 - DNA, 40 bp, Mus musculus
agacagagaaggccagactctgagttgtactggatgatct

SEQ ID NO: 396 - DNA, 40 bp, Mus musculus
ttgaactggcctactgctggaacctcccatggggttgggg

SEQ ID NO: 397 - DNA, 40 bp, Mus musculus
ttagaatcagtaaggagggacccaggctaagaaggcaatc

SEQ ID NO: 398 - DNA, 40 bp, Mus musculus
tggactcagatgtgctagactgagctgtactggatgatct

SEQ ID NO: 399 - DNA, 40 bp, Mus musculus
ccctcacagggaccctcagaaacctcccatggggttgggg

(19) United States
(12) Patent Application Publication
(10) Pub. No.: US 2010/0122358 A1
(43) Pub. Date: May 13, 2010

(54) H-Chain-only antibodies

(75) Inventors: Marianne Brüggemann, Cambridge (GB); Xiangang Zou, Cambridge (GB); Louise Matheson, Cambridge (GB); Michael Osborn, Haverhill (GB)

Correspondence Address: BELL & ASSOCIATES, 58 West Portal Avenue No. 121, SAN FRANCISCO, CA, 94127, US

(73) Assignee: Crescendo Biologics Limited, Cambridge (GB)

(21) Appl. No.: 12/455,913

(22) Filed: Jun. 8, 2009

Related U.S. Application Data
(60) Provisional application No. 61/131,195, filed on Jun. 6, 2008.
(60) Provisional application No. 61/137,502, filed on Jul. 30, 2008.

(51) Int. Cl. C12P 21/00; A01K 67/027; C12N 15/74; C12N 5/10; C12N 5/18; C07K 16/00; A61K 39/395; A61P 35/00

(52) U.S. Cl. 800/4; 800/18; 435/320.1; 435/354; 435/69.6; 435/326; 530/387.2; 424/133.1

(57) ABSTRACT

The invention relates to mice having functionally silenced endogenous lambda (λ) and kappa (κ) L-chain loci, comprising antibody-producing cells in which the C_{H}1 domain is functionally silenced, either via spontaneous processes in somatic antibody-producing cells or due to germline deletion of the C_{H}1 domain. Mice of the invention are capable of producing H-chain-only antibody lacking a functional C_{H}1 domain; transgenic human heavy-chain-only antibodies lacking a functional C_{H}1 domain can be produced following insertion into the mouse of an artificial locus with human heavy chain V, D and J segments and a constant region, which is preferably a modified constant region with alterations in, around or upstream of a C_{H}1 domain and/or removal of a C_{H}1 domain.

### CROSS-REFERENCE TO RELATED APPLICATIONS

This application claims priority from and the benefit of U.S. Provisional Application No. 61/131,195, filed Jun. 6, 2007 and of U.S. Provisional Application No. 61/137,502, filed Jul. 30, 2008, each of the applications identified above is incorporated by reference herein for all purposes.

### COLOR DRAWINGS

The file of this patent contains at least one drawing executed in color. Copies of this patent with color drawing(s) will be provided by the Patent and Trademark Office upon request and payment of the necessary fee.

### FIELD OF THE INVENTION

The present invention relates to heavy (H)-chain-only antibodies, for example produced in light (L)-chain loci-deficient mice, production of the antibodies, and uses thereof. The present invention further relates to a method for the production of a heavy (H)-chain-only immunoglobulin (Ig) A binding molecule in a light (L)-chain loci-deficient mouse, the binding molecule per se, and uses thereof.

### BACKGROUND

Most natural antibodies or immunoglobulins (Ig's) typically comprise two heavy (H-) chains and two light (L-) chains. The H-chains are joined to each other by disulphide bonds located near flexible hinge domains, and each L-chain is associated with the N-terminal part of the C-chain by a disulphide bond. Each L-chain has a variable (V_{L}) and a constant (C_{L}) domain, while each H-chain comprises a variable domain (V_{H}), a first constant domain (C_{H}1), a hinge domain and two or three further constant domains (C_{H}2, C_{H}3 and optionally C_{H}4). In normal dimeric antibodies, interaction of the V_{H} and V_{L} domains forms an antigen binding region, although binding is facilitated by the C_{H}1 domain and parts of the C_{L} domain.

Several different classes of natural Ig have been identified. These classes differ in the constant domains of their H-chains, which in turn affects the function of the Ig. In mammals, the five type of Ig are IgA, IgD, IgE, IgG and IgM. Humans and mice have four IgG subtypes, and humans have two IgA subtypes. IgA comprises three C_{H} domains encoded by C_{α} gene segments, including αC_{H}1 (also termed Cα1), αC_{H}2 (also termed Cα2) and αC_{H}3 (also termed Cα3) genes encoding αC_{H}1, αC_{H}2 and αC_{H}3 domains, respectively. IgA plays a central role in mucosal immunity, which is established after release of IgA from a plasma cell and transport to the mucosal epithelial cell layer. In this layer, polymeric IgA is bound to a polymeric Ig receptor, which, after cleavage, provides the secretory component important for stabilization and conferring resistance to attack by proteases (reviewed in ref. 81). Secretory IgA is dimeric, containing two H₂L₂ units joined by one J chain (82), and is generally abundant in secretions such as milk and colostrum. Serum IgA is present at lower levels in the mouse, mainly in a dimeric form, whereas in humans it is more highly expressed but monomeric.

IgD comprises C_{H} domains encoded by C_{δ} gene segments, is monomeric, and functions as an antigen receptor on B cells, the cells responsible for producing antibodies. IgE has C_{H} domains encoded by C_{ε} gene segments, is also monomeric, and binds to allergens and receptors on mast cells, which triggers the release of cytokines and histamine (allergy response). IgG comprises C_{H} domains encoded by C_{γ} gene segments, is monomeric, and provides most of the antibody-based (humoral) response against pathogens. Finally, IgM has C_{H} domains encoded by C_{μ} gene segments, is pentameric, and is expressed on the surface of B cells and also in a secreted form. Secreted IgM has a role in eliminating pathogens in the early stages of B cell-mediated immunity.

In the mammalian immune system, DNA recombination and surface IgM expression are required for B-lymphocyte development. In bone marrow B cells, D to J_{H} rearrangement is completed at the pre B1 stage. This is followed by V_{H} to DJ_{H} rearrangement in large pre B2 cells, and V_{L} to J_{L} in small pre B2 cells, indicating sequential differentiation events (1-3). At the pre B2 cell stage, replacement of surface-expressed surrogate L-chain by kappa (κ) or lambda (λ) L-chain initiates the process of antibody maturation, which is accompanied by cellular migration and class-switching. Mature B cells undergo further selection and can differentiate into antibody secreting plasma cells or memory B cells bearing different isotypes (IgG, IgA or IgE). Checkpoints during the progression of these regular events ensure that only cells with productive rearrangements advance in differentiation (4). The formation of the B cell receptor (BCR) and its associated chains are regarded as essential to allowing normal B cell development (5). This has been confirmed in mice lacking the H-, L-, Igα or Igβ polypeptide of the BCR (6-8).

Normal Ig expression in B cells involves an ordered succession of gene rearrangements. Exons encoding variable regions of H-chains are constructed in vivo by assembly of V_{H}, diversity (D) and joining (J_{H}) segments, while for L-chains V and J_{L} segments are assembled. During B-cell development, the genes involved in recombinase activity controlling V(D)J rearrangements are specifically expressed at the pre-B cell stage. The rearranged VDJ region is initially transcribed in association with the C_{μ} gene segment, leading to the synthesis of an IgM H-chain. Subsequently, by a process called switch recombination, the C_{μ} gene segment is deleted and the downstream C_{δ} gene segment is used to synthesise an IgD H-chain. The process of isotypic switching continues by bringing further downstream C_{H} (γ, α or ε) gene segments close to the VDJ exon. Switch regions within each of the gene segments are required for switch recombination. In mice, the H-chain gene segment order is 5′-D-J_{H}-C_{μ}-C_{δ}-C_{γ3}-C_{γ1}-C_{γ2b}-C_{γ2a}-C_{ε}-C_{α}-3′. The human H-chain gene segment order is 5′-D-J_{H}-C_{μ}-C_{δ}-C_{γ3}-C_{γ1}-C_{ψε2}-C_{α1}-C_{γ2}-C_{γ4}-C_{ε1}-C_{α2}-3′.

In Tylopoda or camelids (dromedaries, camels and llamas), a major type of Ig, composed solely of paired H-chains (9), is produced in addition to conventional antibodies of paired H- and L-chains (10). The secreted homodimeric H-chain-only antibodies found in these animals use specific V_{H} (V_{H}H) and γ genes which result in a smaller than conventional H-chain, lacking the C(constant)_{H}1 domain. Interestingly, H-chain antibodies are also present in some primitive fish; e.g. the new antigen receptor (NAR) in the nurse shark and the specialized H-chain (COS5) in raffish (11, 12). Again these H-chain Igs lack the C_{H}1-type domain. However, evolutionary analysis has shown that their genes emerged and evolved independently, whereas H-chain genes in camelids evolved from pre-existing genes used for conventional heteromeric antibodies (13). H-chain antibodies can also be found in humans with Heavy Chain Disease (HCD) where the H-chain-only Ig has part of the V_{H} and/or C_{H}1 domain removed (14).

It has also been shown that Tylopoda or camelids (camels, dromedaries and llamas), and recently by us in mice, that H-chain-only IgG antibodies are expressed when the γC_{H}1 exon is removed by splicing of the RNA transcript or DNA deletion, respectively (9, 49, 86). The loss of this exon fits with its putative function of providing a disulphide linkage to the L-chain. Parallels have been drawn to the expression of H-chain-only antibodies in cartilaginous fish, which also lack C_{H}1 or a C_{H}1-type domain (87, 12). These single chains are comprised of a flexible assembly of 3-5 C_{μ} domains, and are part of a large assortment of isotypes of different lengths and function found in lower vertebrates, possibly arising by differential splicing to overcome proteolysis (88).

The synthesis of abnormal Ig has been reported in humans with various immunoproliferative disorders. In the case of heavy-chain disease (HCD) where H-chain-only Ig proteins are produced, lymphoid proliferation is associated with pathological and clinical features. One form of HCD, αHCD, is prevalent in developing countries (84), and accompanied by rapid expansion of B cells producing truncated α H-chain (85). Characterisation of a range of HCD Ig's reveals that most of the abnormal proteins have an isotype not from the most 3′ C_{H} gene segments (such as C_{γ2}-C_{γ4}-C_{α2}) but from the most 5′ gene segments (such as C_{μ}, C_{γ3}, C_{γ1} or C_{α1}). In humans, it is considered that the switch regions of the most 5′ C_{H} gene segments are more susceptible to abnormal deletions than switch regions of the 3′ C_{H} gene segments (43).

Intracellular transport of Ig is dependent on its correct folding and assembly in the endoplasmic reticulum, where single H-chain is chaperoned by non-covalent association with the H-chain binding protein BiP or grp78 (15). The BiP/H-chain complex is formed by virtue of the KDEL sequence at the carboxy terminus of BiP (16) and the C_{H}1 domain of the H-chain. When L-chain displaces BiP Ig can go to the cell surface or be secreted. If C_{H}1, or part of V_{H}, is missing L-chain is no longer required to replace BiP and the H-chain can travel unhindered to the cell surface and be secreted as seen in animals that make H-chain-only antibodies and in HCD.

The present invention arises from the surprising finding (see examples below) that diverse H-chain-only IgG without C_{H}1 is found in the serum of mice deficient in L-chain but without further genetic manipulation, despite compromised B cell development in these mice. We have found that H-chain-only IgGs are produced from naturally- or endogenously-produced transcripts lacking the C_{H}1 exon.

The invention relates to mice having functionally silenced endogenous lambda (λ) and kappa (κ) L-chain loci, comprising antibody-producing cells in which the C_{H}1 domain is functionally silenced, either via spontaneous processes in somatic antibody-producing cells or due to germline deletion of the C_{H}1 domain. Mice of the invention are capable of producing H-chain-only antibody lacking a functional C_{H}1 domain; transgenic human heavy-chain-only antibodies lacking a functional C_{H}1 domain can be produced following insertion into the mouse of an artificial locus with human heavy chain V, D and J segments and a constant region, which is preferably a modified constant region with alterations in, around or upstream of a C_{H}1 domain and/or removal of a C_{H}1 domain. According to a first aspect of the present invention, there is provided a mouse having functionally silenced endogenous lambda (λ) and kappa (κ) L-chain loci, in which the mouse comprises an antibody-producing cell that produces a H-chain-only antibody lacking a functional C_{H}1 domain following in vivo functional silencing of a gene encoding the C_{H}1 domain.

Unlike prior art mice used in the production of H-chain-only antibodies (see for example WO2006/008548), in some embodiments the invention does not require artificial genetic manipulation to functionally silence (for example, by deletion or disruption) the C_{H}1 domain within an H-chain locus prior to insertion of the locus into a mouse for the production of H-chain-only antibodies. Rather, in some embodiments, the invention utilises previously undescribed natural, spontaneous processes in the L-chain deficient mice to functionally silence the C_{H}1 domain.

The mouse having functionally silenced endogenous lambda and kappa L-chain loci may, for example, be made as disclosed in WO03/000737, which is hereby incorporated by reference in its entirety. In WO03/000737 functional silencing of the Igκ locus was achieved by insertion of neo into Cκ (46); functional silencing of the λ locus was by a Cre-IoxP mediated deletion of ˜120 kb encompassing all Cλ genes.

The mouse may also have a functionally silenced endogenous heavy chain locus, for example produced as disclosed in WO04/076618, which is hereby incorporated by reference in its entirety. In WO04/076618 functional silencing of the endogenous heavy chain constant region locus was achieved by Cre-IoxP mediated deletion of the heavy chain constant region genes.

Preferably the mouse is capable of expressing pre-BCR and/or surface display of an endogenous or exogenous IgM.

The mouse of the invention may additionally comprise a nucleic acid construct integrated into the endogenous mouse genome, in which the nucleic acid construct comprises non-murine heavy chain genes from which the H-chain-only antibody is produced.

The non-murine heavy chain genes may be from other vertebrates including mammals such as from a rat or particularly from a human.

One example of a suitable construct comprising human heavy chain genes is the IgH YAC construct disclosed in WO2004/049794 and/or reference 80, which are hereby incorporated by reference in their entirety.

The nucleic acid construct may additionally comprise one or more mouse C_{H} genes, including a C_{H}1 gene. This will allow natural processing mechanisms to functionally silence the C_{H}1 gene to produce a H-chain-only antibody.

The nucleic acid construct may exclude a functional human C_{H}1 gene.

In vivo functional silencing of the C_{H}1 domain gene according to the invention may be achieved by class switch recombination. For reasons elaborated in the specific embodiments, it is understood that the natural mechanism of in vivo functional silencing of C_{H}1 domain genes in L-chain deficient mice is class switch recombination. Class switch recombination has been described in the prior art, for example see references 25, 33 and 52, which are hereby incorporated by reference in their entirety.

According to another aspect of the invention there is provided a transgenic mouse of the invention having a nucleic acid construct integrated in the endogenous mouse genome, in which the nucleic acid construct comprises non-murine vertebrate (for example, human) V, D and J region genes and in which the mouse produces a mouse-non-murine vertebrate (such as mouse-human) chimeric H-chain-only antibody comprising non-murine vertebrate (for example, human) V, D and J domains and one or more mouse C_{H} domains excluding a functional C_{H}1 domain.

In this aspect of the invention, the mouse may recognise mouse C_{H} genes and associated regulatory switch recombination sequences (see below) to allow in vivo functional silencing of the C_{H}1 domain and thereby formation of an H-chain-only antibody.

The non-murine vertebrate (for example, human) V, D and J region genes in the construct may be in non-murine vertebrate (for example, human) or murine (mouse) germline configuration. Non-murine vertebrate (for example, human) germline configuration will be suitable for achieving similar selection and maturation (recombination) events in the V, D and J regions to those found in the non-murine vertebrate (for example, human). Re-positioning of the human V, D and J region genes in the construct to mirror mouse germline configuration will allow efficient selection and maturation (recombination) events of the non-murine vertebrate (for example, human) V, D and J regions within the mouse.

The non-murine vertebrate (for example, human) V, D and J domains of the antibody in this aspect of the invention preferably result from recombination of the non-murine vertebrate (for example, human) V, D and J region genes. The process of somatic hypermutation will allow development of a diverse H-chain-only antibody repertoire.

The nucleic acid construct in the mouse may be integrated upstream of endogenous mouse C_{H} region genes. This will allow in vivo functional silencing of the endogenous C_{H}1 domain gene and formation of the H-chain-only antibody. Alternatively, the nucleic acid construct in the mouse may comprise one or more mouse C_{H} region genes including a C_{H}1 domain gene. Here, in vivo functional silencing of the introduced construct C_{H}1 domain gene allows formation of the H-chain-only antibody. In both cases, the C_{H}1 domain gene may be functionally silenced by class switch recombination.

According to a further aspect of the invention there is provided a transgenic mouse having a nucleic acid construct integrated in the endogenous mouse genome, in which the nucleic acid construct comprises:

(i) non-murine vertebrate (for example, human) heavy chain V, D, J and C genes, for example in non-murine vertebrate (for example, human) or mouse germline configuration, including a non-murine vertebrate (for example, human) C_{H}1 gene; and
(ii) class switch recombination sequences upstream of the non-murine vertebrate (for example, human) C_{H}1 gene.

The class switch recombination sequences may facilitate class switch recombination-mediated functional silencing of the non-murine vertebrate (for example, human) C_{H}1 gene in vivo, thereby allowing production of a non-murine vertebrate (for example, human) H-chain-only antibody in the mouse.

Class switch recombination sequences for use in the invention, for example mouse class switch recombination sequences, are described in the specific embodiments below and are also as known in the art (see for example references 25, 33 and 52, incorporated herein by reference in their entirety). These sequences facilitate the class switch recombination process to allow functional silencing of the C_{H}1 gene by deletion.

The mouse of the invention in one aspect lacks or is deficient in B cell receptor (BCR)-expressing B cells.

The mouse in one aspect does not exhibit lymphoproliferation as seen in human H chain disease (HCD; see reference 14).

The mouse of the invention may be inbred through two or more generations to increase production of the H-chain-only antibodies. We have found in particular that mice with functionally silenced endogenous lambda (λ) and kappa (κ) L-chain loci when bred through successive generations increase the serum level of H-chain-only antibody production, presumably due to selection of antibody transcripts with functionally silenced C_{H}1 domains which produce the expressed antibodies.

In another aspect of the invention there is provided an isolated nucleic acid (for example, a vector such as a BAC, YAC or artificial chromosome) comprising a construct or an antibody as described herein.

According to another aspect of the invention there is provided a host cell comprising the nucleic acid as defined above.

A further aspect of the invention is use of a mouse of the instant invention in the production of an H-chain-only antibody lacking a functional C_{H}1 domain following in vivo functional silencing of a gene encoding the C_{H}1 domain.

Also provided is a method for obtaining an H-chain-only antibody from a mouse, comprising the steps of:

(i) producing a mouse with functionally silenced endogenous lambda and kappa L-chain loci;
(ii) allowing formation in the mouse of an H-chain-only antibody lacking a functional C_{H}1 domain following in vivo functional silencing of a gene encoding the C_{H}1 domain; and
(iii) obtaining the H-chain-only antibody from mouse serum.

A specific embodiment of this aspect is a method for obtaining an IgG-type H-chain-only antibody from a mouse, comprising the steps of:

(i) producing a mouse with functionally silenced endogenous lambda and kappa L-chain loci using the method described in WO03/000737;
(ii) allowing in vivo functional silencing of a gene encoding a C_{H}1 domain in the mouse;
(iii) forming an IgG-type H-chain-only antibody lacking a functional C_{H}1 domain; and
(iv) obtaining the IgG-type H-chain-only antibody from mouse serum.

According to another aspect of the invention there is provided a method for isolating an antibody-producing cell which produces an antigen-specific H-chain-only antibody (for example, an IgG-type H-chain-only antibody), comprising the steps of:

(i) obtaining a mouse with functionally silenced endogenous lambda and kappa L-chain loci;
(ii) immunizing the mouse with an antigen;
(iii) selecting for a cell producing an antigen-specific H-chain-only antibody lacking a functional C_{H}1 domain following in vivo functional silencing of a gene encoding the C_{H}1 domain; and
(iv) isolating the cell selected in step (iii).

In this aspect of the invention, the mouse may be produced using the method described in WO03/000737.

Selection and isolation of the cell may employ flow-cytometry, for example for the identification and isolation of B220^{int/+}, syndecan⁺ spleen-derived plasma cells in which an antigen-specific H-chain-only antibody lacking a functional C_{H}1 domain is produced.

In an alternative aspect, peritoneal cells are selected and isolated.

Another embodiment of this aspect of the invention is a method for isolating an antibody-producing cell which produces an antigen-specific IgG-type H-chain-only antibody, comprising the steps of:

(i) obtaining a mouse with functionally silenced endogenous lambda and kappa L-chain loci using the method described in WO03/000737;
(ii) immunizing the mouse with an antigen;
(iii) isolating sub-populations of cells from secondary lymphoid organs or bone marrow in the mouse;
(iv) screening the sub-populations of cells isolated in step (iii) by RT-PCT using J_{H} to γC_{H}2 amplifications to detect mutant γ H chain transcripts in which the C_{H}1 exon has been deleted; and
(v) selecting those sub-populations of cells screened in step (iv) which have mutant γ H chain transcripts; and
(vi) isolated cells selected in step (v).

The antibody-producing cell of this aspect of the invention may be isolated from a secondary lymphoid organ. For example, the secondary lymphoid organ may be a non-splenic organ, for example any of the group consisting of: lymph node, tonsil, and mucosa-associated lymphoid tissue (MALT), including gut-associated lymphoid tissue (GALT), bronchus-associated lymphoid tissue (BALT), nose-associated lymphoid tissue (NALT), larynx-associated lymphoid tissue (LALT), skin-associated lymphoid tissue (SALT), vascular-associated lymphoid tissue (VALT), and/or conjunctiva-associated lymphoid tissue (CALT). In one embodiment, the antibody-producing cell is a peritoneal cell.

In the methods of the invention, the produced H-chain-only antibody may be non-murine vertebrate (for example, human) or a mouse-non-murine vertebrate (for example, human) chimera. Where a mouse-non-murine vertebrate chimeric (for example, a mouse-human chimeric) H-chain-only antibody is produced, the antigen-specificity-determining regions (defined by the VDJ domains) may be non-murine vertebrate (for example, human), with the C regions being of mouse origin.

Also provided is an isolated antibody-producing cell obtainable using the method of the invention.

Further provided according to the invention is a hybridoma obtainable by fusion of an antibody-producing cell as defined herein with a B-cell tumor line cell. We have found (see Example 2) that H-chain-only antibody production is not dependent on the presence of a mouse spleen, so in certain embodiments of the invention the antibody-producing cell used to form the hybridoma is a non-splenic secondary lymphoid organ cell (see above). Well known methods of generating and selecting single clone hybridomas for the production of monoclonal antibodies may be adapted for use in the present invention.

The invention in another aspect provides an H-chain-only antibody lacking a functional C_{H}1 domain following in vivo functional silencing of a gene encoding the C_{H}1 domain, or a fragment of the antibody.

The antibody of the invention may be produced in mouse having functionally silenced endogenous lambda and kappa L-chain loci.

The antibody of the invention may be in an isolated and purified form. The antibody may be isolated and/or characterised using methods well known in the art. Once characterised, the antibody or the fragment thereof may be manufactured using recombinant or synthetic methods, also well known in the art. For applicable prior art methods, see references listed below.

The antibody may be modified to increase solubility, for example by genetic engineering of one or more genes encoding the antibody.

The antibody of the invention may be specific to an antigen. The antibody may be engineered to be a bi- or multi-valent antibody with one or more specificities.

The antibody of the invention may be a monoclonal antibody.

The antibody of the invention may be an IgG-like antibody or an IgM-like antibody (as exemplified below).

In one aspect of the invention, the H-chain-only antibody has the structure V_{H}DJ_{H}-hinge-C_{H}2-C_{H}3. Each domain of the antibody may be of non-murine vertebrate (for example, human) or of mouse origin, or the antibody may be chimeric (for example where the V_{H}DJ_{H} part of the structure is non-murine vertebrate, such as human, and the hinge-C_{H}2-C_{H}3 part of the structure is murine).

The antibody of the invention in preferred embodiments lacks endogenous gross alteration of the V_{H} regions as seen in human HCD (see reference 14).

The antibody of the invention in certain embodiments does not include one or more or all camelid V_{H}H-specific mutations found in V_{H} to V_{H}H substitutions in framework 2 (i.e. Val37Phe, Gly44Glu, Leu45Arg and Trp47Gly). As shown below, such mutations are not required for production of H-chain-only antibodies in L-chain-deficient mice.

The antibody of the invention may be used as a diagnostic, prognostic or therapeutic imaging agent. The antibody may additionally or alternatively be used as an intracellular binding agent, or an abzyme.

The antibody of the invention may be for use as a medicament in the treatment of a disease.

The antibody of the invention may be for use in the manufacture of a medicament in the treatment of a disease.

Also provided is a medicament comprising an antibody of the invention. The medicament will typically be formulated using well-known methods prior to administration into a patient.

In a further aspect of the invention there is provided a method of treating a disease, comprising the step of administering a medicament of the invention to a patient in need of same.

Diseases which are susceptible to treatment using an antibody include: wound healing, cell proliferative disorders, including neoplasm, melanoma, lung, colorectal, osteosarcoma, rectal, ovarian, sarcoma, cervical, oesophageal, breast, pancreas, bladder, head and neck and other solid tumors; myeloproliferative disorders, such as leukemia, non-Hodgkin lymphoma, leukopenia, thrombocytopenia, angiogenesis disorder, Kaposis' sarcoma; autoimmune/inflammatory disorders, including allergy, inflammatory bowel disease, arthritis, psoriasis and respiratory tract inflammation, asthma, immunodisorders and organ transplant rejection; cardiovascular and vascular disorders, including hypertension, oedema, angina, atherosclerosis, thrombosis, sepsis, shock, reperfusion injury, and ischemia; neurological disorders including central nervous system disease, Alzheimer's disease, brain injury, amyotrophic lateral sclerosis, and pain; developmental disorders; metabolic disorders including diabetes mellitus, osteoporosis, and obesity, AIDS and renal disease; infections including viral infection, bacterial infection, fungal infection and parasitic infection, pathological conditions associated with the placenta and other pathological conditions.

As used herein, the term “antibody” refers to both a naturally produced antibody which may be generated in response to an antigen and also where appropriate to a synthetic binding molecule which mimics the binding ability of a natural antibody, for example with modified binding or other pharmacological properties. Where appropriate, the term also encompasses antibody fragments, for example antigen-binding or effector antibody fragments.

Production of the H-chain only antibody according the invention may include expression from an antibody-producing cell, either by expression on the cell surface or by secretion (i.e. release of antibody from the cell).

As used herein, the term “in vivo” refers to an endogenous or a natural (non-engineered) process. The endogenous or natural process occurs spontaneously.

The term “mouse” used herein encompasses in further aspects of the invention other vertebrates such as mammals, preferably non-human mammals such as other rodents including rats.

As used herein, a non-murine vertebrate includes mammals such as a rat and a human, particularly a human.

The present invention further relates to a new type of H-chain-only binding molecule surprisingly found in L-chain deficient mice.

According to an aspect of the present invention, there is provided a method for producing an H-chain-only IgA binding molecule in a mouse, comprising the steps of:

(i) obtaining an L-chain deficient mouse with functionally silenced endogenous lambda and kappa L-chain loci; and
(ii) allowing formation in the L-chain deficient mouse of an H-chain-only IgA binding molecule lacking a functional αC_{H}1 domain.

There are no published examples of the occurrence H-chain-only IgA binding molecules in healthy animals, only in humans with αHCD. Thus H-chain-only IgA have not been reported in camelids, which produce H-chain-only IgG, nor in elasmobranchs (sharks, skates and rays), where H-chain-only antibodies can comprise a variable number of C_{μ} domains (50, 88). The production of H-chain only IgA binding molecules according to the invention is also unexpected given the 3′ downstream location of the C_{α} gene segment compared with other Ig isotypes in mice. The invention allows production of H-chain-only IgA binding molecules (of murine or other origin) in a mouse, for example in stable and in relatively high amounts.

Furthermore, unlike prior art mice suggested for use in the production of H-chain-only antibodies (see for example WO2006/008548), this aspect of the invention does not require artificial genetic manipulation to functionally silence (for example, by deletion) the αC_{H}1 domain within an αH-chain locus prior to insertion of the locus into a mouse for the production of H-chain-only IgA binding molecules.

The L-chain deficient mouse used in the method is in one aspect relatively healthy compared with a corresponding mouse without functionally silenced endogenous lambda and kappa L-chain loci, when kept under the same conditions (for example, pathogen-free conditions). The L-deficient mouse preferably does not show equivalent pathological and/or clinical symptoms seen in humans with αHCD (see ref. 43). For example, the L-deficient mouse may not exhibit lymphoproliferation.

The mouse having functionally silenced endogenous lambda and kappa L-chain loci may, for example, be made as described in WO03/000737, which is hereby incorporated by reference in its entirety. In WO03/000737 functional silencing of the Igκ locus was achieved by insertion of neo into Cκ((46); functional silencing of the λ locus was by a Cre-IoxP mediated deletion of ˜120 kb encompassing all Cλ genes.

The method of the invention may comprise a further step (iii) of isolating the H-chain-only IgA binding molecule.

In the method, the H-chain-only IgA binding molecule may be formed following in vivo functional silencing of a gene encoding the αC_{H}1 domain. The H-chain-only IgA binding molecule may be formed following in vivo deletion of all or a part of the gene encoding the αC_{H}1 domain. All or a part of the gene encoding the αC_{H}1 domain may be deleted in vivo by imprecise class-switch recombination. Additionally or alternatively, all or a part of the gene encoding the αC_{H}1 domain may be deleted in vivo due to one or more point mutations, out of frame reading, an incorrect stop codon and/or a splice site alteration.

In vivo deletion of all or a part of the gene encoding the αC_{H}1 domain in one aspect of the invention is not accompanied by DNA insertion. In this aspect, the deletion mechanism is distinct from H-chain only antibody formation in αHCD where in-frame DNA insertions have been detected (43).

The H-chain-only IgA binding molecule may be produced in the L-chain deficient mouse at a level 0.2-2 times, for example about 0.25, 0.5, 1.0, 1.25, 1.5 or 1.75 times, that of a normal IgA antibody produced in a corresponding mouse without functionally silenced endogenous lambda and kappa L-chain loci. The level of production of the H-chain-only IgA binding molecule in the L-chain deficient mice is surprisingly high, as demonstrated in the specific embodiments below.

The L-chain deficient mouse may be at least 2.5 months old, for example at least 3 to 14 months old, such as about 5, 6, 9, 11, 12, 13 or 14 months old. We have found that generally older mice produce higher levels of H-chain-only IgA binding molecule.

The H-chain-only IgA binding molecule may be produced in a bone marrow cell, a mucosal cell (for example from a lamina propria or epithelial layer) and/or a spleen lymphocyte cell of the mouse. For example, the molecule may be produced in a spleen lymphocyte IgA⁺B220⁺ cell.

The H-chain-only IgA binding molecule produced according to the method may comprise a functional V_{H} domain. In an aspect of the invention, the H-chain-only IgA binding molecule does not have a deletion and/or an insertion in any V_{H} domains (as found in αHCD H-chain-only IgA antibodies).

The H-chain-only IgA binding molecule produced according to the method may comprise a functional αC_{H}2 domain and/or a functional αC_{H}3 domain.

The H-chain-only IgA binding molecule produced according to the method may comprise functional D and J_{H} domains.

The H-chain-only IgA binding molecule produced according to the method in one aspect has functional domains in the following order: V_{H}-D-J_{H}αC_{H}2-αC_{H}3.

The H-chain-only IgA binding molecule produced according to the method may be a monomer. The monomer may have a single antigen binding site.

Alternatively, the H-chain-only IgA binding molecule produced according to the method may be a multimer for example a dimer or a tetramer. Each H-chain-only IgA binding molecule of the multimer may have a single antigen binding site. The multimer may thus have binding sites for more than one antigen.

The H-chain-only IgA binding molecule of the multimer may be associated with one or more J chains.

The H-chain-only IgA binding molecule produced according to the method may be a non-murine vertebrate binding molecule or a mouse-non-murine vertebrate chimeric binding molecule. The non-murine vertebrate may be a human.

The L-chain deficient mouse used in the method may additionally have all or part of its endogenous H-chain locus functionally silenced (for example produced as disclosed in WO04/076618, which is hereby incorporated by reference in its entirety). In WO04/076618 functional silencing of the endogenous heavy chain constant region locus was achieved by Cre-IoxP mediated deletion of the heavy chain constant region genes.

Preferably the mouse is capable of expressing preBCR and/or surface display of an endogenous or exogenous IgM.

The L-chain deficient mouse used in the method may additionally comprise a nucleic acid construct integrated into the endogenous mouse genome, in which the nucleic acid construct comprises non-murine H-chain genes from which the H-chain-only IgA binding molecule is produced. The non-murine H-chain genes may be from other vertebrates including mammals such as from a rat or particularly from a human. One example of a suitable construct comprising H-chain genes, optionally with modification as suggested herein, is the IgH YAC construct disclosed in WO2004/049794, which is hereby incorporated by reference in its entirety.

The invention accordingly encompasses a method of producing a human H-chain-only IgA molecule in the L-chain deficient mouse.

In one embodiment there is provided a method for obtaining an H-chain-only IgA binding molecule from a mouse, comprising the steps of:

(i) producing a mouse with functionally silenced endogenous lambda and kappa L-chain loci using the method described in WO03/000737;
(ii) allowing in vivo functional silencing of a gene encoding a αC_{H}1 domain in the mouse;
(iii) forming an H-chain-only IgA binding molecule lacking a functional αC_{H}1 domain; and
(iv) obtaining the H-chain-only IgA binding molecule from mouse serum, milk and/or saliva.

The nucleic acid construct may additionally comprise one or more mouse C_{H} genes, including an αC_{H}1 gene. This will allow natural processing mechanisms to functionally silence the αC_{H}1 gene to produce an H-chain-only binding molecule. The nucleic acid construct may exclude a functional human αC_{H}1 gene.

The L-chain deficient mouse used in the method may have integrated into its genome a nucleic acid construct comprising non-murine vertebrate V, D and J region genes and in which the mouse produces a mouse-non-murine vertebrate chimeric H-chain-only IgA binding molecule having non-murine vertebrate V_{H}, D and J_{H} domains and one or more mouse αC_{H} domains other than a functional αC_{H}1 domain.

In this aspect of the invention, the mouse may recognise mouse C_{H} genes and associated regulatory switch recombination sequences (see below) to allow in vivo functional silencing of the C_{H}1 domain and thereby formation of an H-chain-only antibody.

The non-murine vertebrate (for example, human) V, D and J region genes in the construct may be in non-murine vertebrate (for example, human) or murine (mouse) germline configuration. Non-murine vertebrate (for example, human) germline configuration will be suitable for achieving similar selection and maturation (recombination) events in the V, D and J regions to those found in the non-murine vertebrate (for example, human). Re-positioning of the human V, D and J region genes in the construct to mirror mouse germline configuration will allow efficient selection and maturation (recombination) events of the non-murine vertebrate (for example, human) V, D and J regions within the mouse.

The non-murine vertebrate (for example, human) V, D and J domains of the binding molecule in this aspect of the invention preferably result from recombination of the non-murine vertebrate (for example, human) V, D and J region genes. The process of somatic hypermutation will allow development of a diverse αH-chain-only antibody repertoire.

The nucleic acid construct in the L-chain deficient mouse used in the method may be integrated upstream of endogenous mouse αC_{H} region genes. This will allow in vivo functional silencing of the endogenous αC_{H}1 gene and formation of the H-chain-only IgA binding molecule. Alternatively, the nucleic acid construct in the mouse may comprise one or more mouse αC_{H} region genes including an αC_{H}1 gene. Here, in vivo functional silencing of the introduced construct αC_{H}1 gene allows formation of the H-chain-only IgA binding molecule. In both cases, the αC_{H}1 gene may be functionally silenced by class switch recombination.

The L-chain deficient mouse used in the method may have a nucleic acid construct integrated in the endogenous mouse genome, in which the nucleic acid construct comprises:

(i) non-murine vertebrate (for example, human) heavy chain V, D, J and C genes, for example in non-murine vertebrate (for example, human) or mouse germline configuration, including a non-murine vertebrate (for example, human) αC_{H}1 gene; and
(ii) class switch recombination sequences upstream of the non-murine vertebrate (for example, human) αC_{H}1 gene.

The class switch recombination sequences may facilitate class switch recombination-mediated functional silencing of the non-murine vertebrate (for example, human) αC_{H}1 gene in vivo, thereby allowing production of a non-murine vertebrate (for example, human) H-chain-only IgA binding molecule in the mouse.

Class switch recombination sequences for use in the invention, for example mouse class switch recombination sequences, are as known in the art (see for example references 8 and 24, incorporated herein by reference in their entirety). These sequences facilitate the class switch recombination process to allow functional silencing of the αC_{H}1 gene by deletion.

The L-chain deficient mouse used in the method may be inbred through two or more generations to increase production of the H-chain-only IgA binding molecules.

The L-chain deficient mouse used in the method preferably comprises a functional C, gene segments to allow pre-BCR B cell development and/or surface IgM expression.

In another aspect of the invention, there is provided an H-chain-only IgA binding molecule obtainable according to the method as described herein, or a functional fragment or derivative thereof.

The H-chain-only IgA binding molecule (including a functional fragment or derivative thereof) may have features as described above and below.

The H-chain-only IgA binding molecule may be in an isolated and/or substantially pure form. The binding molecule may be isolated and/or characterised using methods well known in the art. Once characterised, the binding molecule may be manufactured using recombinant or synthetic methods, also well known in the art.

The H-chain-only IgA binding molecule may be modified to increase solubility, for example by genetic engineering of one or more genes encoding the H-chain-only IgA binding molecule.

The H-chain-only IgA binding molecule of the invention may be specific to an antigen. The binding molecule may be engineered to be a bi- or multi-valent binding molecule with one or more specificities.

The H-chain-only IgA binding molecule of the invention may be monoclonal.

The H-chain-only IgA binding molecule may be non-human or part-human.

The H-chain-only IgA binding molecule may be obtained from mouse serum or secreted fluid (for example, milk, saliva, tears and/or sweat). Alternatively, the molecule may be obtained from the mouse faeces and/or urine.

The H-chain-only IgA binding molecule of the invention in certain embodiments does not include one or more or all camelid V_{H}H-specific mutations found in V_{H} to V_{H}H substitutions in framework 2 (i.e. Val37Phe, Gly44Glu, Leu45Arg and Trp47Gly). Such mutations are not required for production of the binding molecule in L-chain-deficient mice.

The H-chain-only IgA binding molecule of the invention in certain embodiments does not include extended CDR3 region found in camelid H-chain-only antibodies (97, 40, 41, 42).

The invention encompasses a human H-chain-only IgA molecule obtainable according to methods described herein, other than known human H-chain-only IgA mutant proteins associated with αHCD (as described in references 84, 85, 43, which are incorporated herein by reference in their entirety).

The H-chain-only IgA binding molecule of the invention may be used as a screening agent, a diagnostic agent, a prognostic agent or a therapeutic imaging agent. The binding molecule may additionally or alternatively be used as an intracellular binding agent, or an abzyme. Accordingly, use of H-chain-only IgA binding molecule of the invention as a screening agent, a diagnostic agent, a prognostic agent, a therapeutic imaging agent, an intracellular binding agent or an abzyme is also within the scope of the invention.

Another aspect of the invention provides an H-chain-only IgA binding molecule as defined herein for use as a medicament.

Also provided is an H-chain-only IgA binding molecule as defined herein for use in the manufacture of a medicament for the treatment of a disease.

Further provided is an H-chain-only IgA binding molecule as defined herein for use in the discovery of a medicament. Use of the binding molecule in the discovery of a medicament is also encompassed.

Also provided according to the invention is an H-chain-only IgA binding molecule obtainable according to the invention method and modified, improved and/or evolved using an in vitro display system.

The invention further provides an H-chain-only IgA binding molecule-producing cell obtainable from an L-chain deficient mouse as defined herein. The cell may for example be a bone marrow cell, a mucosa cell, or a spleen lymphocyte cell. The cell may be a spleen lymphocyte IgA⁺ B220⁺ cell. As elaborated in the specific embodiments below, we have found that such a cell exhibits a novel B cell receptor.

According to another aspect of the invention there is provided a method for isolating an antibody-producing cell which produces an antigen-specific H-chain-only IgA binding molecule, comprising the steps of:

(i) obtaining an L-chain deficient mouse with functionally silenced endogenous lambda and kappa L-chain loci;
(ii) immunising the mouse with an antigen;
(iii) selecting for a cell producing an antigen-specific H-chain-only IgA binding molecule lacking a functional αC_{H}1 domain following in vivo functional silencing of a gene encoding the αC_{H}1 domain; and
(iv) isolating the cell selected in step (iii).

In this aspect of the invention, the L-chain deficient mouse may be produced using the method described in WO03/000737.

Selection and isolation of the cell may employ flow-cytometry, for example for the identification and isolation of B220⁺, syndecan⁺ spleen-derived cells in which an antigen-specific H-chain-only IgA binding molecule lacking a functional αC_{H}1 domain is produced.

Another embodiment of this aspect of the invention is a method for isolating an antibody-producing cell which produces an antigen-specific H-chain-only IgA binding molecule, comprising the steps of:

(i) obtaining an L-chain deficient mouse with functionally silenced endogenous lambda and kappa L-chain loci using the method described in WO03/000737;
(ii) immunising the mouse with an antigen;
(iii) isolating sub-populations of cells from spleen, bone marrow and/or mucosa in the mouse;
(iv) screening the sub-populations of cells isolated in step (iii) by RT-PCT using J_{H} to αC_{H}2 amplifications to detect mutant α H chain transcripts in which the αC_{H}1 exon has been deleted; and
(v) selecting those sub-populations of cells screened in step (iv) which have mutant a H chain transcripts; and
(vi) isolated a cell selected in step (v).

Further provided is an H-chain-only IgA binding molecule-producing hybridoma obtainable by fusion of a B-cell tumor line cell with the H-chain-only IgA binding molecule-producing cell as defined herein. Well known methods of generating and selecting single clone hybridomas for the production of monoclonal antibodies may be adapted for use in the present invention.

In another aspect of the invention, there is provided a medicament comprising an H-chain-only IgA binding molecule or functional fragment thereof as defined herein. The medicament may be formulated using well-known methods prior to administration into a patient.

The medicament may for example be formulated with a pharmaceutically or therapeutically acceptable excipient or carrier. Such excipients or carriers include a solid or liquid filler, diluent or encapsulating substance which does not interfere with the effectiveness or the biological activity of the H-chain-only binding molecule and which is not toxic to the host, which may be either humans or animals, to which it is administered. Depending upon the particular route of administration, a variety of pharmaceutically acceptable carriers such as those well known in the art may be used. Non-limiting examples include sugars, starches, cellulose and its derivatives, malt, gelatin, talc, calcium sulfate, vegetable oils, synthetic oils, polyols, alginic acid, phosphate buffered solutions, emulsifiers, isotonic saline, and pyrogen-free water.

Also provided is a method of treating a disease, comprising the step of administering a medicament as defined above to a patient in need of same.

The medicament may be administered by the route selected from the group consisting of orally, intramuscularly, intravenously, intradermally, cutaneously, topically, locally, ocularly and inhalation. Other suitable modes of administration are also contemplated according to the invention. For example, administration of the medicament may be via subcutaneous, direct intravenous, slow intravenous infusion, continuous intravenous infusion, intravenous or epidural patient controlled analgesia (PCA and PCEA), intrathecal, epidural, intracistemal, intraperitoneal, transdermal, transmucosal, buccal, sublingual, transmucosal, intranasal, intra-atricular, intranasal or rectal routes. The medicament may be formulated in discrete dosage units and can be prepared by any of the methods well known in the art of pharmacy.

All suitable pharmaceutical dosage forms are contemplated. Administration of the medicament may for example be in the form of oral solutions and suspensions, tablets, capsules, lozenges, effervescent tablets, transmucosal films, suppositories, buccal products, oral mucoretentive products, topical creams, ointments, gels, films and patches, transdermal patches, abuse deterrent and abuse resistant formulations, sprays, sterile solutions suspensions and depots for parenteral use, and the like, administered as immediate release, sustained release, delayed release, controlled release, extended release and the like.

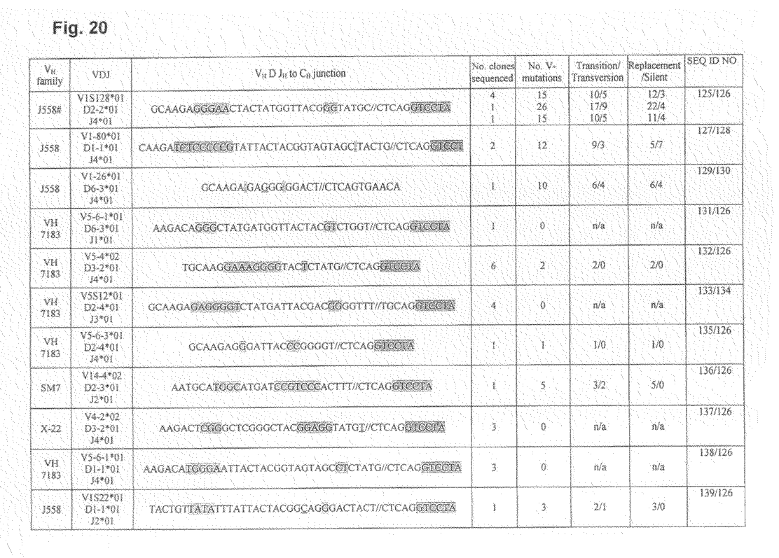

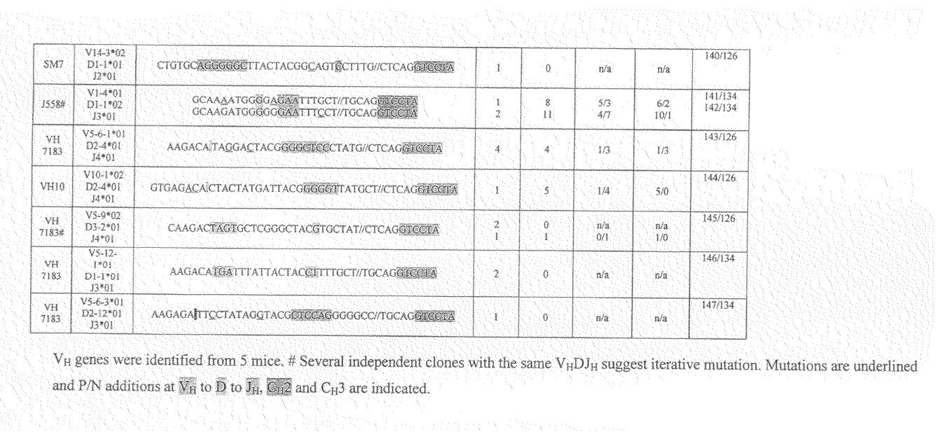

Further provided according to the invention is an isolated nucleic acid (for example, a vector such as an expression vector, a BAC, a YAC or an artificial chromosome) encoding an H-chain-only IgA binding molecule as defined herein. The nucleic acid may comprise or contain any novel sequence disclosed herein (see for example in Tables 5 and 6 and/or FIGS. 20 to 22), or a nucleic acid with at least 50% sequence identity, for example at least 60%, 70%, 80%, 90%, 95%, 96%, 97%, 98% or 99% sequence identity, thereto.

Sequence identity between nucleotide sequences can be determined by comparing an alignment of the sequences. When an equivalent position in the compared sequences is occupied by the same base, then the molecules are identical at that position. Scoring an alignment as a percentage of identity is a function of the number of identical amino acids or bases at positions shared by the compared sequences. When comparing sequences, optimal alignments may require gaps to be introduced into one or more of the sequences to take into consideration possible insertions and deletions in the sequences. Sequence comparison methods may employ gap penalties so that, for the same number of identical molecules in sequences being compared, a sequence alignment with as few gaps as possible, reflecting higher relatedness between the two compared sequences, will achieve a higher score than one with many gaps. Calculation of maximum percent identity involves the production of an optimal alignment, taking into consideration gap penalties.

Suitable computer programs for carrying out sequence comparisons are widely available in the commercial and public sector. Examples include the MatGat program (Campanella et al., 2003, BMC Bioinformatics 4: 29), the Gap program (Needleman & Wunsch, 1970, J. Mol. Biol. 48: 443-453) and the FASTA program (Altschul et al., 1990, J. Mol. Biol. 215: 403-410). MatGAT v2.03 is freely available from the site “http://bitincka.com/ledion/matgat/” and has also been submitted for public distribution to the Indiana University Biology Archive (IUBIO Archive). Gap and FASTA are available as part of the Accelrys GCG Package Version 11.1 (Accelrys, Cambridge, UK), formerly known as the GCG Wisconsin Package. The FASTA program can alternatively be accessed publically from the European Bioinformatics Institute (http://www.ebi.ac.uk/fasta) and the University of Virginia (http://fasta.biotech.virginia.edu/fasta_www/cgi). FASTA may be used to search a sequence database with a given sequence or to compare two given sequences (see http://fasta.bioch.virginia.edu/fasta_www/cgi/search_frm2.cgi). Typically, default parameters set by the computer programs should be used when comparing sequences. The default parameters may change depending on the type and length of sequences being compared. A sequence comparison using the MatGAT program may use default parameters of Scoring Matrix=Blosum50, First Gap=16, Extending Gap=4 for DNA, and Scoring Matrix=Blosum50, First Gap=12, Extending Gap=2 for protein. A comparison using the FASTA program may use default parameters of Ktup=2, Scoring matrix=Blosum50, gap=−10 and ext=−2.

A polypeptide encoded by the nucleic acid as defined herein is also encompassed by the invention.

In another aspect there is provided a host cell comprising the nucleic acid of the invention.

As used herein, the term “binding molecule” refers to an antibody produced in vivo by an animal such as mouse, generated for example in response to an antigen, and also where appropriate to a synthetic binding molecule which mimics the binding ability of an antibody, for example with modified binding or other pharmacological properties. Where appropriate, the term also encompasses functional binding molecule fragments, for example antigen-binding or effector antibody fragments, and/or functional derivates thereof.

As used herein, the term “in vivo” refers to an endogenous or a natural (non-engineered) process. The endogenous or natural process occurs spontaneously.

The term “in vivo functional silencing of a gene encoding the C_{H}1 domain” includes deletion of all or part of the gene encoding the C_{H}1 domain, such that no functional protein can be expressed from the domain. Suitably, “in vivo functional silencing of a gene encoding the C_{H}1 domain” occurs spontaneously by class switch recombination.

Functional silencing of the light chain loci may be achieved by disruption (e.g., by insertion into the locus), or deletion of all or part of the loci, such that no functional protein can be expressed from the loci.

The term “mouse” used herein encompasses in further aspects of the invention other vertebrates such as mammals, preferably non-human mammals such as other rodents including rats.

As used herein, a non-murine vertebrate includes mammals such as a rat and a human, particularly a human.

Somatic alterations leading to C_{H}1 deletion occur at low frequency. This is a limiting step in H-chain-only IgG production, however L-chain deficient mice homozygous in the germline for deletion or disruption of a C_{H}1 exon, such as a γ C_{H}1 exon or alpha C_{H}1 exon, allow H-chain-only antibodies, such as H-chain-only monoclonal antibodies, with defined specificities to be produced.

Accordingly, in an alternative embodiment, the present invention also provides an L-chain deficient mouse having functionally silenced endogenous lambda and kappa L-chain loci and lacking a functional C_{H}1 domain in the germline. Preferably the lambda and kappa L-chain loci are silenced by disruption, such as by an insertion, or by deletion of all or part of the locus such that light chains cannot be expressed from the loci. Preferably the C_{H}1 domain is disrupted (e.g., by insertion), or fully or partially deleted from the germline such that the C_{H}1 domain can not be expressed. Preferably the C_{H}1 domain is a gamma or alpha C_{H}1 domain. Preferably the mouse is homozygous for deletion and/or disruption of the lambda light chain locus, kappa light chain locus and C_{H}1 domain.

The mouse having functionally silenced endogenous lambda and kappa L-chain loci may, for example, be made as disclosed in WO03/000737, which is hereby incorporated by reference in its entirety. In WO03/000737 functional silencing of the Igκ locus was achieved by insertion of neo into Cκ (46); functional silencing of the λ locus was by a Cre-IoxP mediated deletion of ˜120 kb encompassing all Cλ genes.

The mouse may also have a functionally silenced endogenous heavy chain locus, for example produced as disclosed in WO04/076618, which is hereby incorporated by reference in its entirety. In WO04/076618 functional silencing of the endogenous heavy chain constant region locus was achieved by Cre-IoxP mediated deletion of the heavy chain constant region genes.

Preferably the mouse is capable of expressing preBCR and/or surface display of an endogenous or exogenous IgM.

The mouse of the invention in this aspect, lacking a functional C_{H}1 gene in the germline, may additionally comprise a nucleic acid construct integrated into the endogenous mouse genome, in which the nucleic acid construct comprises non-murine heavy chain genes from which the H-chain-only antibody is produced.

The non-murine heavy chain genes may be from other vertebrates including mammals such as from a rodent, like rat or rabbit, or particularly from a human.

One example of a suitable construct comprising human heavy chain genes is the IgH YAC construct disclosed in WO2004/049794 and/or reference 80, which are hereby incorporated by reference in their entirety.

According to this aspect of the invention there is provided a transgenic L-chain deficient mouse having functionally silenced endogenous lambda and kappa L-chain loci and lacking a functional C_{H}1 domain in the germline, preferably lacking a functional αC_{H}1 domain or γC_{H}1 domain, and having a nucleic acid construct integrated in the endogenous mouse genome, in which the nucleic acid construct comprises non-murine vertebrate (for example, human) V, D and J region genes and in which the mouse produces a mouse-non-murine vertebrate (such as mouse-human) chimeric H-chain-only antibody comprising non-murine vertebrate (for example, human) V, D and J domains and one or more mouse C_{H} domains excluding a functional C_{H}1 domain.

The invention further provides a method for producing a H-chain-only immunoglobulin, preferably a H-chain-only immunoglobulin G or A, in an L-chain deficient mouse having functionally silenced endogenous lambda and kappa L-chain loci and lacking a functional C_{H}1 domain, preferably lacking a functional gamma C_{H}1 domain or lacking a functional alpha C_{H}1 domain, comprising the steps of:
(i) providing an L-chain deficient mouse having functionally silenced endogenous lambda and kappa L-chain loci and lacking a functional CH1 domain, and
(ii) allowing formation in said L-chain deficient mouse of a H-chain only immunoglobulin lacking a functional C_{H}1 domain.

The method may comprise the further step (iii) of isolating the H-chain only antibody.

The H-chain only antibody may be produced in response to antigen challenge, such as immunisation with a specific antigen.

In an aspect there is provided a cell from a L-chain deficient mouse having functionally-silenced endogenous lambda and kappa L-chain loci and lacking a functional C_{H}1 domain, said cell being capable of expressing a H-chain-only immunogloblulin, preferably the C_{H}1 domain is a gamma or alpha C_{H}1 domain and the H-chain-only immunogloblulin is IgG or IgA respectively.

In an aspect there is provided a hybridoma cell obtainable by fusion of a B-cell tumor line cell with a cell from a L-chain deficient mouse having functionally silenced endogenous lambda and kappa L-chain loci and lacking a functional C_{H}1 domain, said cell being capable of expressing a H-chain-only immunogloblulin, preferably the H-chain-only immunogloblulin is IgG or IgA, i.e. the C_{H}1 domain is a gamma C_{H}1 domain or an alpha C_{H}1 domain.

Particular non-limiting embodiments of the present invention will now be described below with reference to the following drawings, in which:

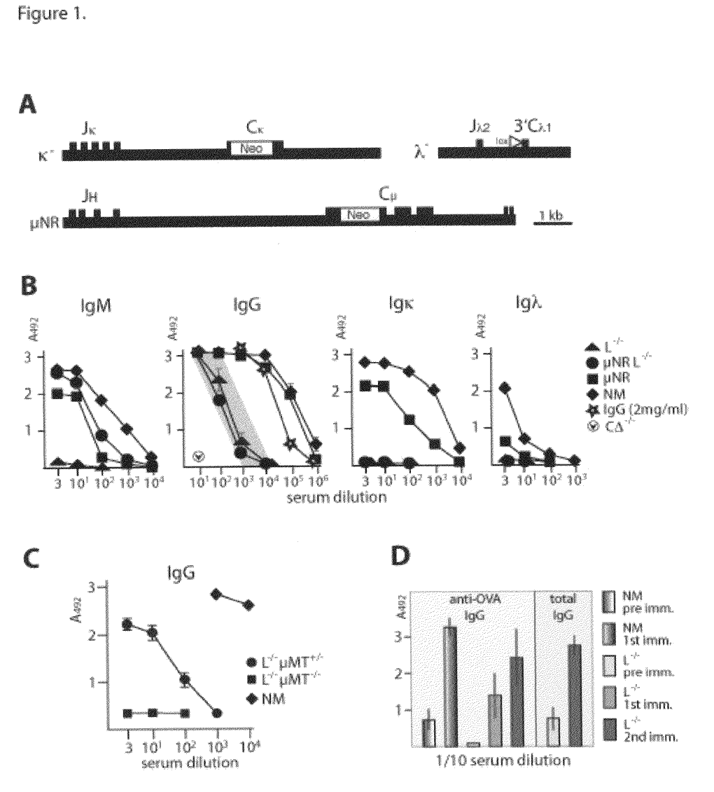

FIG. 1 shows antibody expression in mice without L-chain. (A) κ⁻ mice carry an Igκ locus disabled by insertion of neo into Cκ (46); λ⁻ mice carry a Cre-IoxP mediated deletion of ˜120 kb encompassing all Cλ genes (7); and μNR mice have a neomycin gene (neo) inserted into Cμ exons 1 and 2, and express truncated μ H-chains (17). (B) The level of H-chain Ig in serum from un-immunized mice was titrated in ELISA by binding to antibodies against IgM, IgG, Igκ and Igλ. In L^{-/-} (κ^{-/-}λ^{-/-}) mice 20-100 μg/ml H-chain IgG without L-chain was produced (indicated by the shaded area). μNRL^{-/-} mice produce a similar level of IgG in addition to truncated IgM. In normal mice (NM) ˜10 mg/ml IgG was produced. Purified IgG (DB3, ref 47) served as a standard, and serum from animals with removed C genes, CΔ mice (45) was used as a negative control. (C) L-chain deficient mice homozygous for μMT (L^{-/-}μMT^{-/-}) do not express H-chain IgG in serum whilst in L^{-/-} (heterozygous) μMT^{+/-} mice concentrations were similar to those of L^{-/-} mice. At least 5 mutant mice from separate litters were compared with the standard deviation indicated when >+0.2. (D) Immunizations (1^{st} nd 2^{nd} imm.) with ovalbumin show specific antibody responses and an increase in total IgG (pre imm. compared to post imm.). Groups of mice contained at least 6 animals and standard deviations for IgG are shown when individual serum titrations diverged more than 10%;

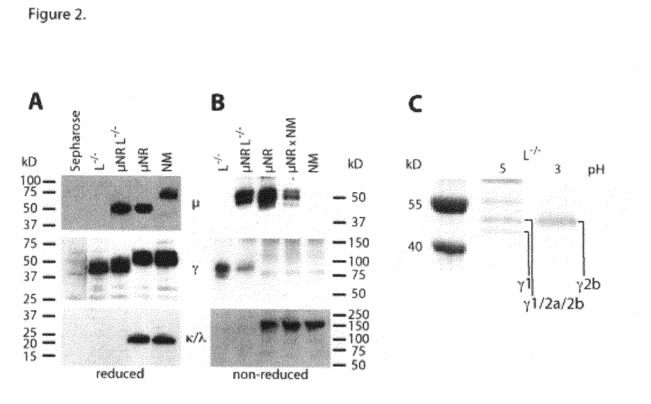

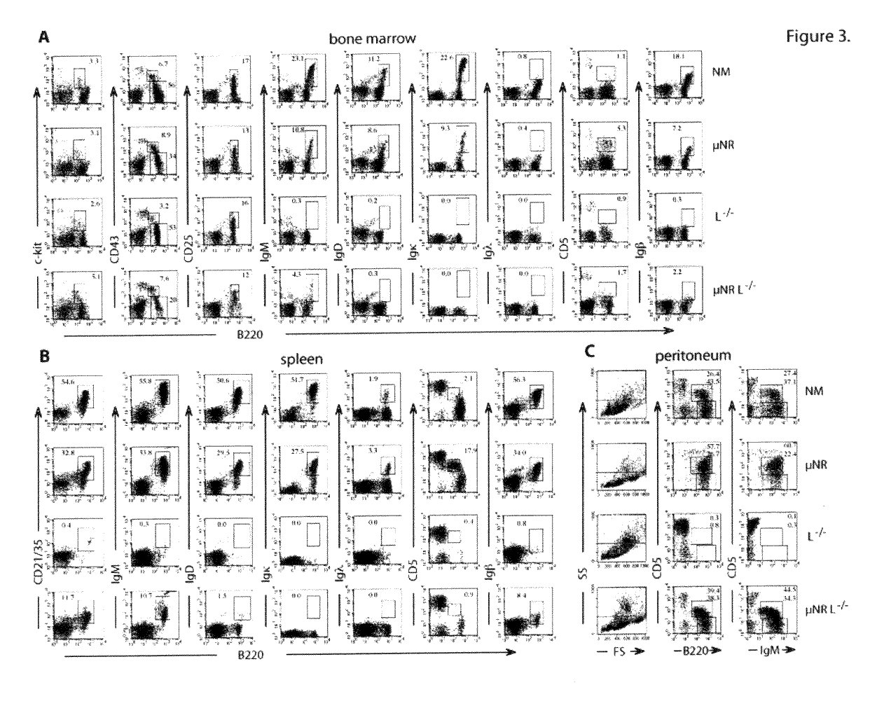

FIG. 2 shows Western blot detection of H-chain-only Ig. Serum antibodies from L^{-/-}, μNRL^{-/-}, μNR, μNR×NM (a heterozygous μNR animal) and normal mice (NM), were purified by incubation with anti-mouse Ig coupled to Sepharose, separated on Ready-Gels and visualized with antibodies against μ, γ and κ and λ L-chain (27, 17). (A) Reducing conditions revealed 44-48 kD bands for γ H-chains in L^{-/-} mice and no μ H-chain or L-chain (κ and λ). μNRL^{-/-} mice showed the same size γ bands in addition to the μ specific band characteristic of the μNR background (17). (B) Under non-reducing conditions γ H-chains from L^{-/-} and μNRL^{-/-} mice associate as dimers of 88-96 kD. Truncated IgM bands, only found in μNRL^{-/-} and μNR mice, are largely monomeric (17). Pentameric IgM (˜900 kD) does not enter the gel and the strong signal of conventional IgG above 150 kD is not shown for μNR, μNR×NM and normal mouse serum (NM). Antibody-coupled Sepharose served as a negative control. (C) For isotype identification of H-chain Ig, serum antibodies from L^{-/-} mice were bound to protein-A, eluted at pH 5 and 3 and size separated on SDS-PAGE. γ1, γ2b and a mixture of γ1/γ2a/γ2b were identified by mass-spectrometry in the bands indicated after trypsin digest. Individual isotypes were identified by between 5 and 9 fragments each with sequences corresponding to hinge, C_{H}2 and C_{H}3 exons but not C_{H}1. For V_{H} sequences framework and CDR regions were identified for genes from the following families: VH7183 (EVQLVESGGDLVKPGGSLK, NTLYLQMSSLK, LVESGGGLVK, NNLYLQMSSLK, EVQLVESGGGLVKPGGSLK), VGAM3.8 and/or J558 (ASGYTFTDYSMHWVK), J558 (EVQLQQSGPELVKPGASVK), J558 and/or SM7 (QSGAELVRPGASVK), SM7 (EVQLQPSGAELVKPGASVK, LSCTASGFNIK) and J606 (LLESGGGLVQPGR). The size of mol wt standards is shown in kD;

FIG. 3 shows generation and maintenance of small numbers of mature B-cells in L^{-/-} mice despite a developmental block at the pre B2 to immature transition stage. Flow cytometry analysis of (A) bone marrow, (B) spleen and (C) peritoneal cells from normal mice (NM), μNR, λ^{-/-} and μNRL^{-/-} mice using antibodies against lymphocyte surface markers: c-kit, CD43, CD25, IgM, IgD, Igκ, Igλ, CD5, Igβ and CD21/35. The profiles are representative for results from at least 5 different animals each using established lymphocyte gate parameters by plotting forward (FS) against side (SS) scatter (20);

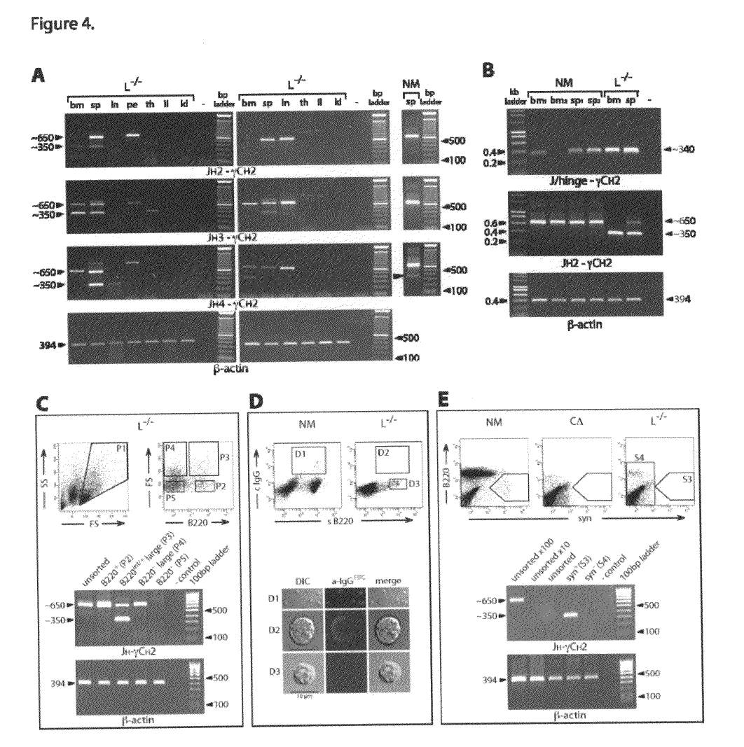

FIG. 4 shows identification of cells that generate H-chain products. (A) RT-PCR amplification from J_{H}2, 43 or J_{H}4 to γC_{H}2 using RNA prepared from total bone marrow (bm), spleen (sp), lymph nodes (ln), peritoneum (pe), thymus (th), ileum (il) and kidney (ki) cells from 2 L^{-/-} mice and spleen from normal mouse (NM). γ H-chain bands of reduced size (˜350 bp, [symbol] indicated in NM) are present in lymphoid tissue, sometimes accompanied by the full size product (˜650 bp). β-actin served as a reference (25 cycles). (B) RT-PCR amplification of bone marrow (bm) and spleen (sp) from normal and L^{-/-} mice using a J/hinge oligo, specific for J to hinge joins that lack C_{H}1 (J_{H}1, J_{H}2 or J_{H}4 and γ2a or γ2b), in combination with the γC_{H}2^{c} oligo. In comparative control reactions J_{H}2 to γC_{H}2^{a} and β-actin (21 cycles) was amplified. (C) For the analysis of spleen cells by FACS and RT-PCR from L^{-/-} mice the lymphocyte gate, established by forward (FS) and side scatter (SS), was set to include large cells (P1). These cells were collected (P2-P4) according to their staining profiles for B220. Large B220⁺ cells (P3) show a γ H-chain RT-PCR band, from J_{H}4 to γC_{H}2, of reduced size (˜350 bp) lacking C_{H}1 whilst other cell fractions contain a normal size H-chain transcript (˜650 bp). PCR reactions were normalized using β-actin. (D) Surface staining for B220 and cytoplasmic staining for IgG showed in confocal images that H-chain antibody producing B-cells are of larger size (D2). DIC denotes Differential Interference Contrast and the size bars are 10 μm. (E) Surface staining for syndecan (syn) (CD138) identified a population (S3) only expressing H-chain transcripts without C_{H}1 in L^{-/-} mice. Syn⁺ cells from NM mice, which are lacking in CΔ mice, established the gate for the cell sort. Normalized RT-PCR reactions (32 cycles) were carried out with the sensitivity and specificity being verified by increased levels of unsorted spleen cells (10×, 100×). Control reactions without DNA (−) are indicated. The data are representations using different mice in at least 3 independent experiments giving very similar results;

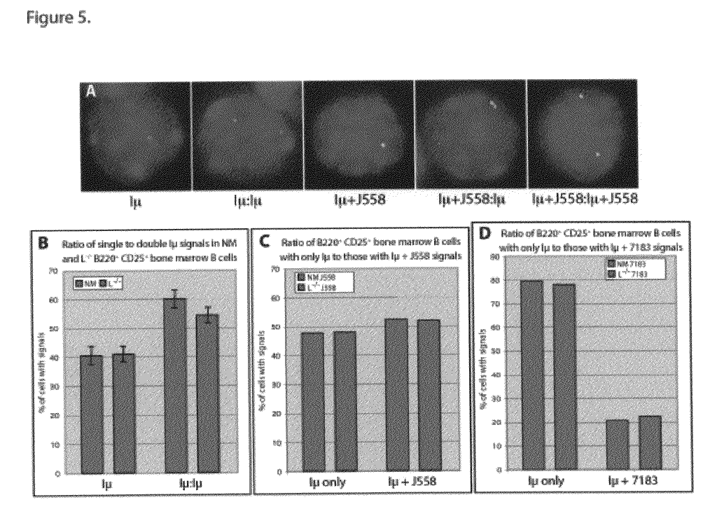

FIG. 5 shows RNA-FISH to assess the transcriptional activity of the H-chain alleles and their V_{H}-gene usage. Detection with an Iμ probe indicated whether one or both of the IgH loci was actively transcribing, and detection with a J558 or, separately, a 7183 probe revealed the V_{H} gene usage of V_{H}DJ_{H} rearranged alleles. Cells from normal (NM) and L^{-/-} mice were analyzed in parallel. (A) Representative signal combinations detected for Iμ (red) and J558 (green) transcripts in sorted B220⁺ CD25⁺ L^{-/-} bone marrow cells. (B-D) Comparison of signal ratios in sorted B220⁺ CD25⁺ bone marrow cells from normal and L^{-/-} mice stained for (B) Iμ, (C) Iμ and J558 and (D) Iμ and 7183 transcripts. Standard deviation was calculated from 4 separate experiments whilst representative plots (C, D) were at least repeated once with similar results;

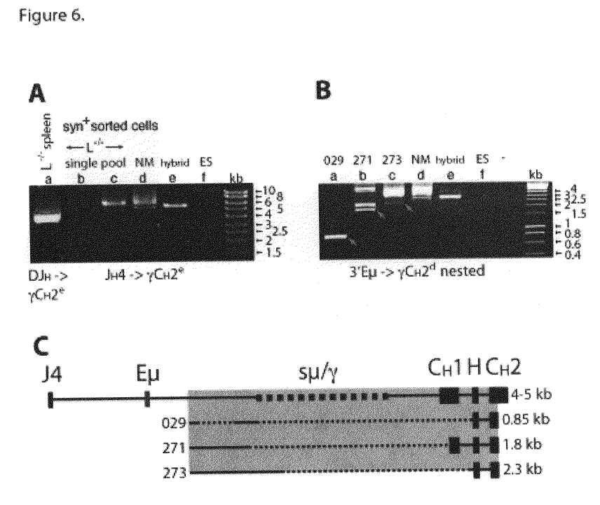

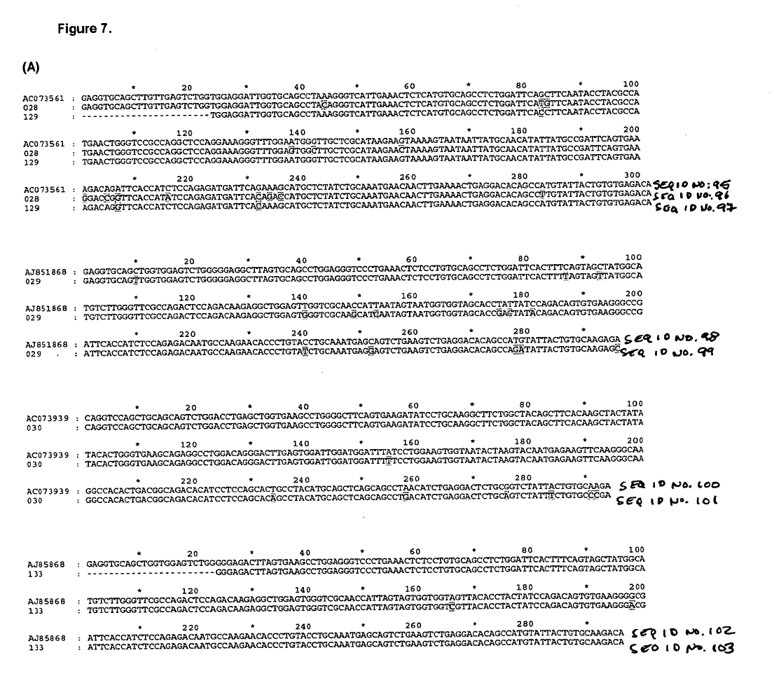

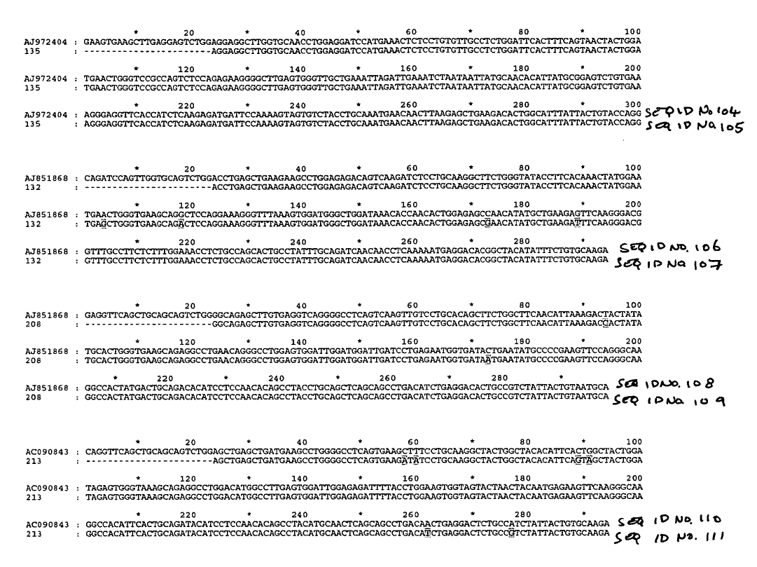

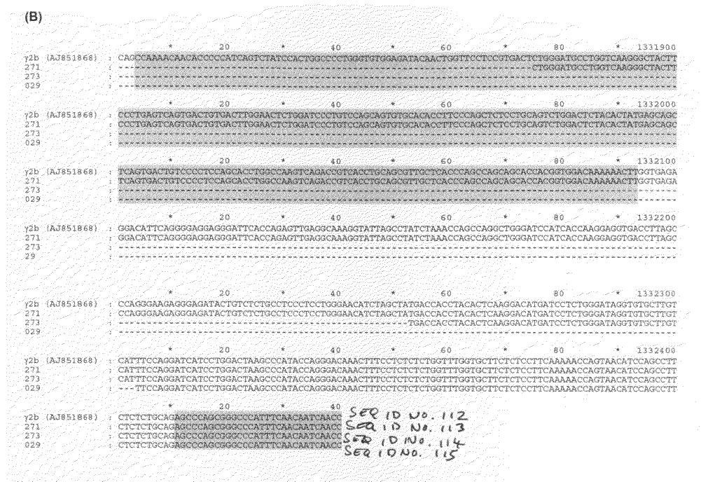

FIG. 6 shows long range PCR identified class-switch deletions in C_{H}1. DNA preparations from one total spleen and sorted syn⁺ spleen cells from four L^{-/-} mice were analyzed. (A) PCR amplifications from DJ_{H} to γC_{H}2^{e} (40 cycles), using a primer (VDJ029) based on the H-chain sequences obtained by RT-PCR (left), or from J_{H}4 to γC_{H}2^{e} (20 cycles) (right). In the reactions cell aliquots from one (single) and three (pool) L^{-/-} mice were used. (B) Nested PCR (28 cycles) of first round products (a-f) from Eμ to γC_{H}2^{d} with cloned products indicated by arrows. Controls were a γ2a hybridoma (hybrid), ES cell DNA and amplification without DNA (−). (C) Map of the amplified genomic region from J_{H}4 to Cγ exons C_{H}1, hinge (H) and C_{H}2. Cloning and sequencing of PCR products showed deletions of large parts of the switch region and some or all of C_{H}1. Clone numbers and sizes [029 (0.85 kb), 271 (1.8 kb) and 273 (2.3 kb)], from 3′Eμ to γC_{H}2^{d}, are indicated (with sequence information compiled in FIG. 7 and Table 4);

FIG. 7 shows V_{H} cDNA and genomic Cg H-chain Ig sequences obtained in Example 1 below. (A) Matches to the closest V_{H} region were performed using IMGT/V-QUEST (79). Numbering according to Kabat. Shading indicates differences. (B) Genomic Cγ H-chain sequences identified by long range PCR shown in FIG. 6 identified break points within γ2b. The shaded boxes mark exon 1 (C_{H}1, top) and hinge (5′ region, bottom);

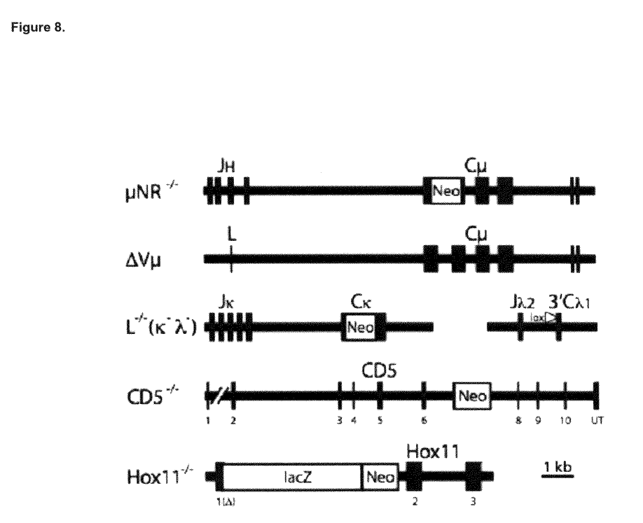

FIG. 8 shows gene alterations used to analyze L-chain independent antibody expression. μNR mice have a targeted insertion of the neomycin gene (neo) in Cμ exons 1 and 2, and express truncated μ H-chains. The ΔVμ construct, expressed as a transgene, was obtained by removal of the rearranged V_{H}DJ_{H} but retention of the leader (L) exon, which permits splicing to Cμ. In L^{-/-} (κ⁻λ⁻) mice the Igκ locus is disabled by insertion of neo into Cκ and λ⁻ mice carry a Cre-IoxP mediated deletion of ˜120 kb encompassing all Cλ genes. The CD5 (Ly-1) antigen has been inactivated by homologous integration of a neo gene replacing exon 7, the transmembrane domain, which prevents surface deposition. The Hox11 homeobox gene, essential for spleen development, was silenced by targeted integration of lacZ-neo into exon 1;

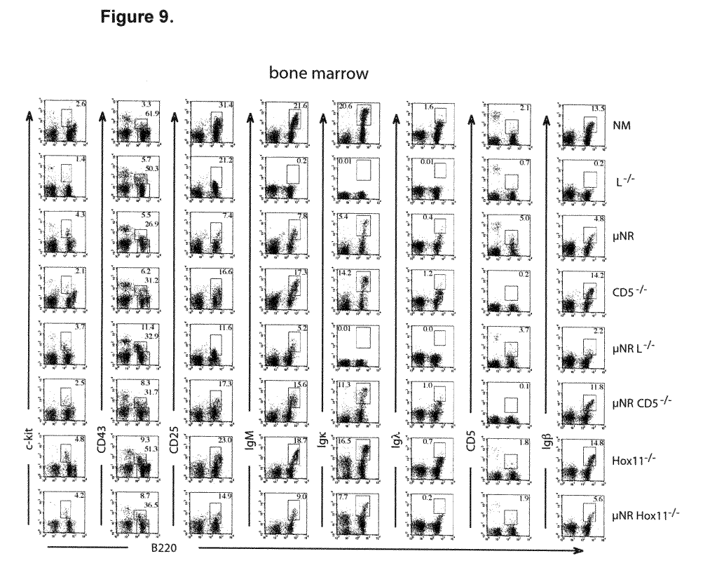

FIG. 9 shows surface expression of L-chain deficient immature B cells with incomplete BCR. Bone marrow cells, prepared from normal (NM), L^{-/-}, μNR, CD5^{-/-}, μNR L^{-/-}, μNR CD5^{-/-}, Hox11^{-/-} and μNR Hox11^{-/-} mice, were stained with antibodies against lymphocyte surface markers c-kit, CD43, CD25, IgM, Igκ, Igλ, CD5 and Igβ and analyzed by flow cytometry. The profiles are representative for results from at least 5 different animals each using established lymphocyte gate parameters;

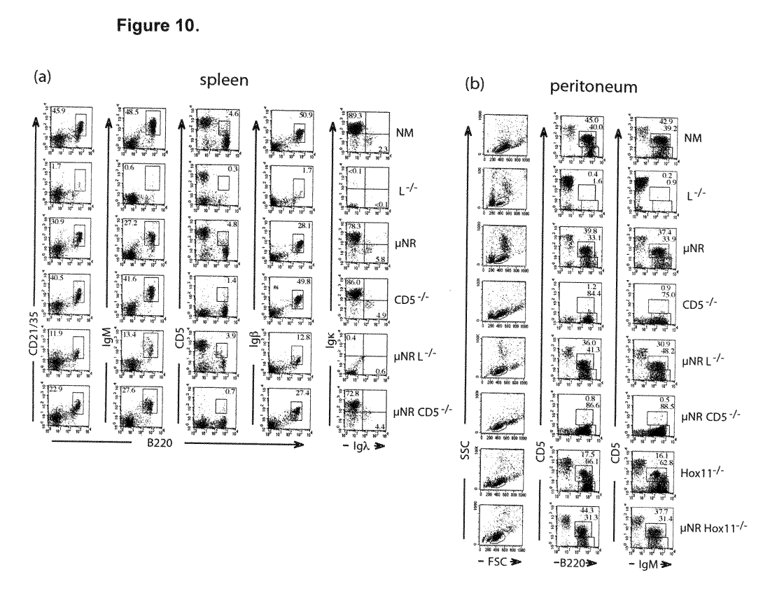

FIG. 10 shows developmental progression of mature lymphocyte populations devoid of IgL. Stainings and flow cytometry analysis of spleen (a) and peritoneal cells (b) were carried out as in FIG. 9, with the addition of an antibody against CD21/35. Plotting forward (FS) against side (SS) scatter shows that the conventional lymphocyte gate is applicable for all the lines analyzed although size, shape and number of accompanying cells may vary;

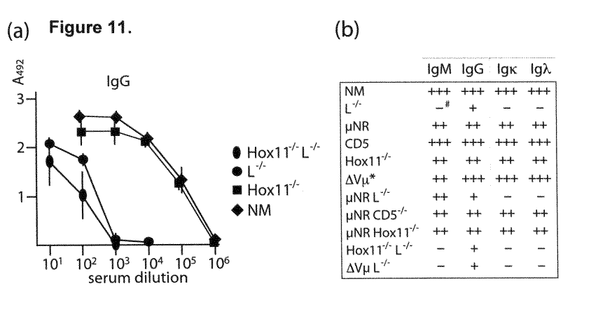

FIG. 11 shows production of serum Ig despite compromised BCR and lymphocyte deficit. (a) Antibody titration by ELISA shows that H-chain IgG levels in L^{-/-} and Hox11^{-/-} L^{-/-} mice are largely independent of spleen development. (b) Titration results using anti-mouse Ig for the detection of IgM, IgG, Igκ and Igλ antibodies: +++, corresponds to conventional levels found for normal mice in our barrier facilities (>1 mg/ml Ig); ++, somewhat reduced (0.3-0.8 mg/ml Ig); +, reduced but easily detectable (10-200 μg/ml Ig); and −, non-detectable levels (<0.1 μg/ml Ig) compared to IgG, IgM and IgL (κ and λ) levels for normal mice kept under the same conditions. *ΔVμ mice produce low levels of human IgM in serum and none was found in ΔVμ L^{-/-} mice. ^{#}L^{-/-} mice sometimes have low levels of truncated μ-chain in the serum, which increases in some older mice. Serum from at least 5 mice (3 mice in the case of ΔVμ), all kept under pathogen-free conditions, were used for the analysis; and

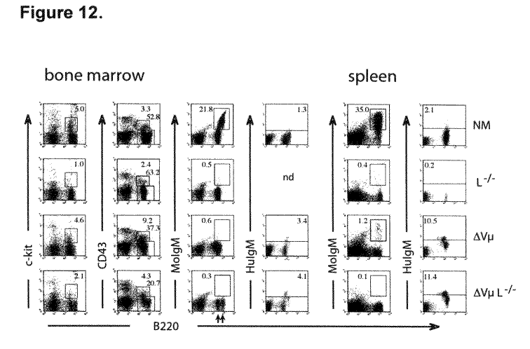

FIG. 12 shows surface expression of μ HCD protein without L-chain. Bone marrow and spleen cells from normal (NM), L^{-/-}, ΔVμ and ΔVμ L^{-/-} mice were stained with antibodies against c-kit, CD43, mouse IgM and human IgM, which identified B cell development and μ expression on the cell surface by flow cytometry. Arrows indicate the two distinct B220⁺ populations.

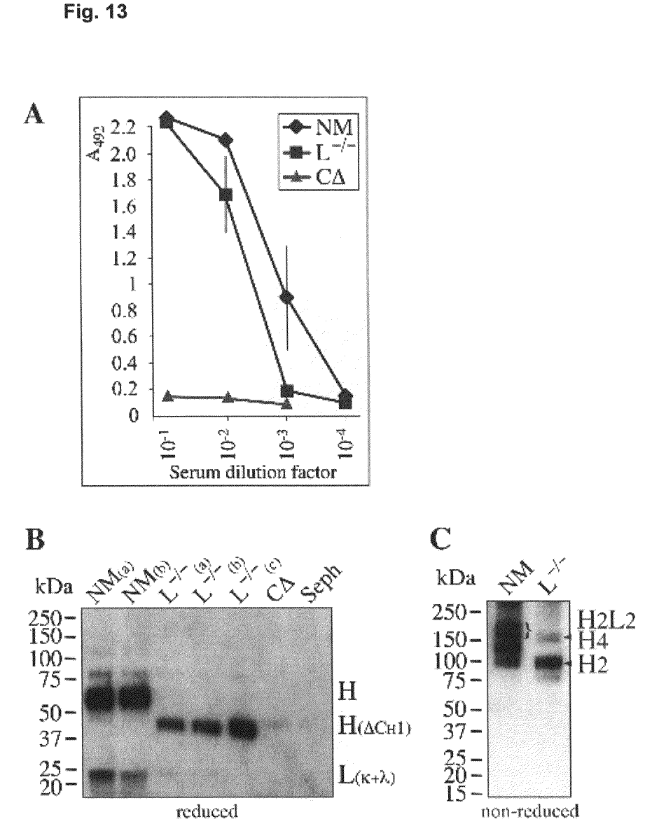

FIG. 13 shows a graph and two Western blots illustrating serum IgA without L-chain is of reduced molecular weight. (A) The level of H-chain-only IgA from 8^{-/-} mice kept under pathogen-free conditions was titrated in ELISA by binding to anti-IgA; mice expressing high titers of IgA were selected to show how similar serum IgA levels can be to normal mice. Plots were obtained by calculating the means and bars indicate the standard deviation when serum titers diverged >10%. Control serum was from 8 normal (NM) and two constant region deletion mice (CΔ) (45). (B,C) Serum antibodies from several mice (a-c) were purified by capturing with anti-Ig coupled to sepharose, separated on Ready-Gels and visualized with biotinylated antibodies against IgA, κ and λ-1, 2 and 3 chains (B) or anti-IgA alone (C). Separation under reducing conditions (B) revealed an α H-chain band of ˜46 kDa in the serum from L^{-/-} mice and no L-chain. Anti-Ig coupled sepharose and serum from constant region deletion (CΔ) mice served as negative controls. Separation under non-reducing conditions (C) revealed that α H-chains can associate as dimers (H2) of ˜92 kDa and H4 multimers. Normal mice (NM) control serum shows the expected size range, including H2L2, with the smaller products due to inherent incomplete disulphide formation of IgA (102).

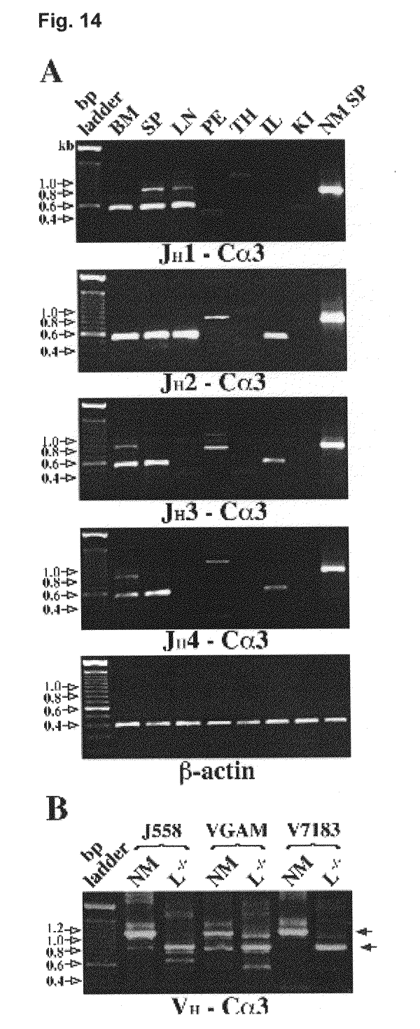

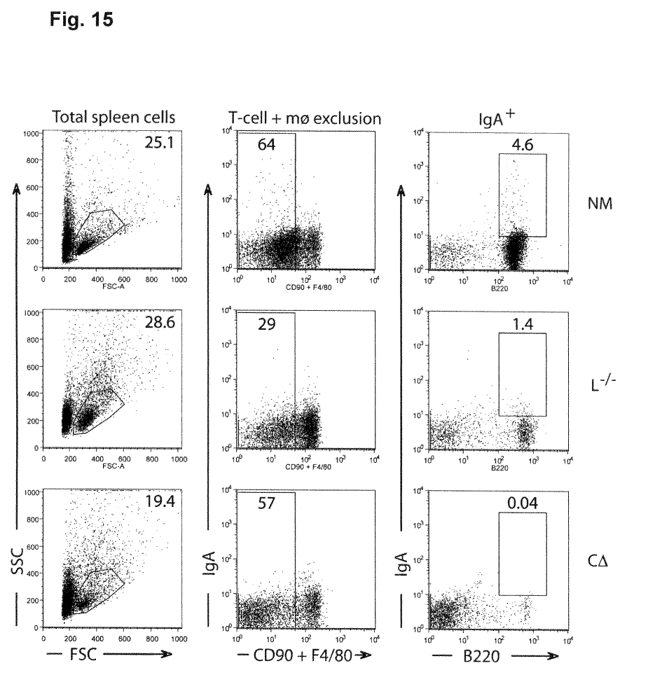

FIG. 14 shows gels allowing identification of reduced size a transcripts in lymphoid tissues. (A) RT-PCR amplification from J_{H}1, J_{H}2, J_{H}3 or J_{H}4 to Cα3 using RNA prepared from total bone marrow (BM), spleen (SP), lymph nodes (LN), peritoneum (PE), thymus (TH), ileum (IL) and kidney (KI) from L^{-/-} mice and spleen from normal mice (NM SP). α H-chain bands of reduced size, ˜550 bp, are present in lymphoid tissues from L^{-/-} mice, sometimes accompanied by the full size product of ˜850 bp, typical for normal mice. β-actin served as a control to ascertain the use of equal amounts cDNA template. (B) V_{H} (J558, VGAM and V7183 oligos) to Cα3 amplification of NM and L^{-/-} spleen c-DNA shows extensive V-gene usage in shorter products (˜880 bp, bottom arrow) compared to normal products (˜1150 bp, top arrow).

FIG. 15 shows flow cytometry analysis of surface IgA⁺ lymphocytes in L^{-/-} spleen. Flow cytometry analysis of spleen cells from L^{-/-} mice compared to normal (NM) mice and constant region deletion (CΔ) animals. Numbers show the percentage of cells in the respective gate. Gating spleen lymphocytes (left) and exclusion of T cells and macrophages (middle) identified for L^{-/-} mice 1.4% of IgA⁺ B220⁺ lymphocytes (right). The analysis is a representative presentation of one L^{-/-} animal with high IgA titer as shown in FIG. 13.

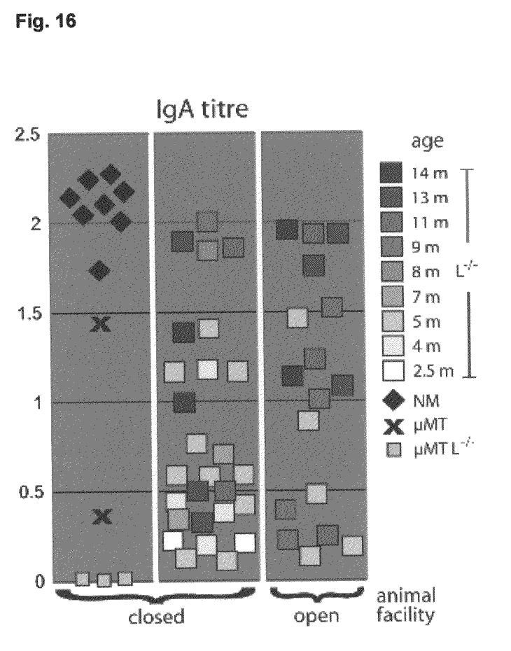

FIG. 16 is a graph showing comparison of age and environment on H-chain-only IgA production. The serum titer of L^{-/-} mice of different age, housed in open or closed (pathogen-free) animal facilities, and control animals (NM, μMT¹⁶ and μMT L^{-/-} homozygous cross-breeds) was assessed by comparative ELISA at 1/100 dilution. Only some older L^{-/-} mice, independent of the housing facility, express high IgA levels. μMT mice produce variable levels as previously identified²¹ and no IgA could be detected in serum from μMT L^{-/-} mice.

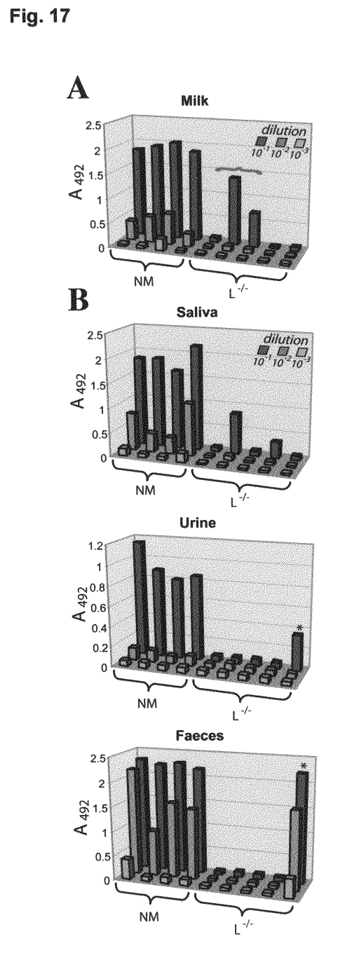

FIG. 17 shows histograms of IgA expression in bodily fluids. Excreted IgA in milk, saliva, urine and faeces from several normal and L^{-/-} mice was titrated by ELISA. The shading of the bar indicates the dilution as shown (10⁻¹, 10⁻² and 10⁻³). (A) Milk was taken from different pups from the same litter, indicated by a bracket, whilst other pups were from different mothers. (B) The experiments shown are representative for the finding that only one or two animals of a group of at least five tested in parallel excrete H-chain-only IgA. Animals are not matched except were indicated by an asterisk (*) and this animal is also the mother of the three siblings in (A).

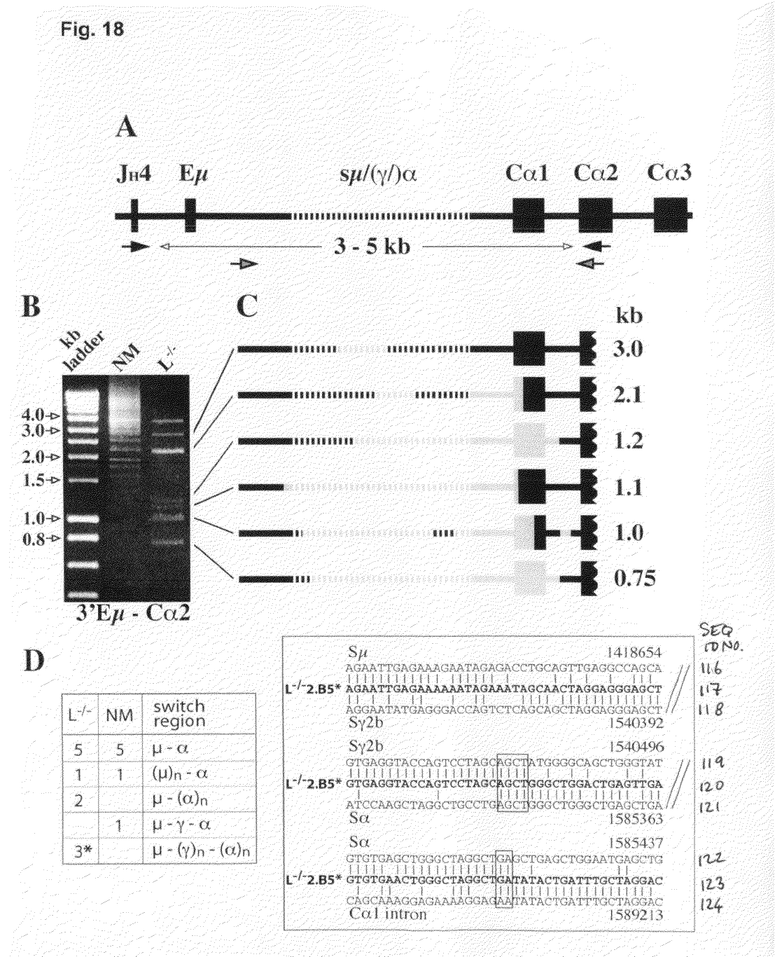

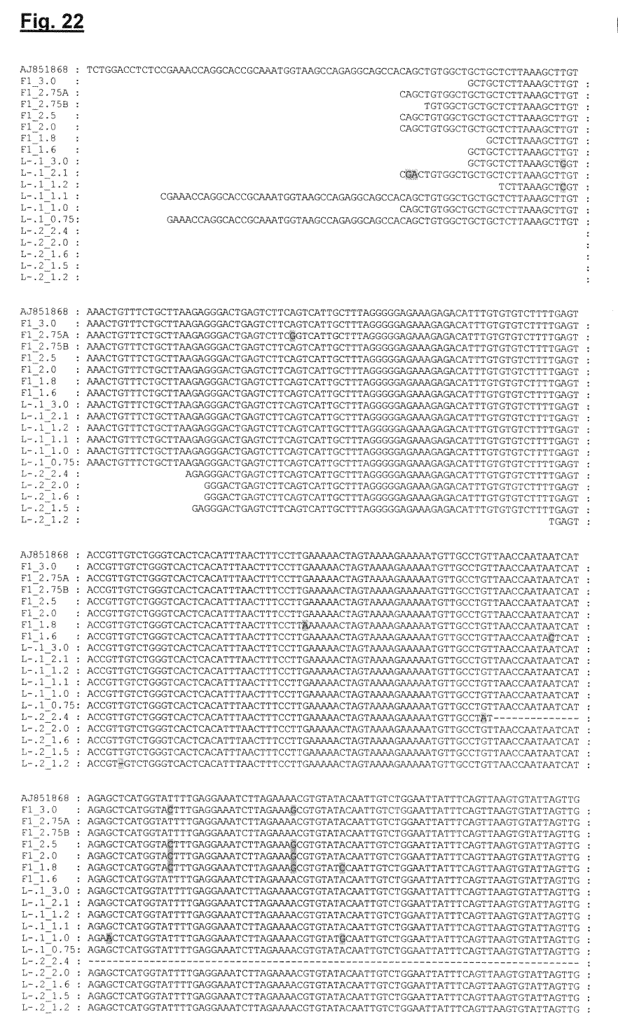

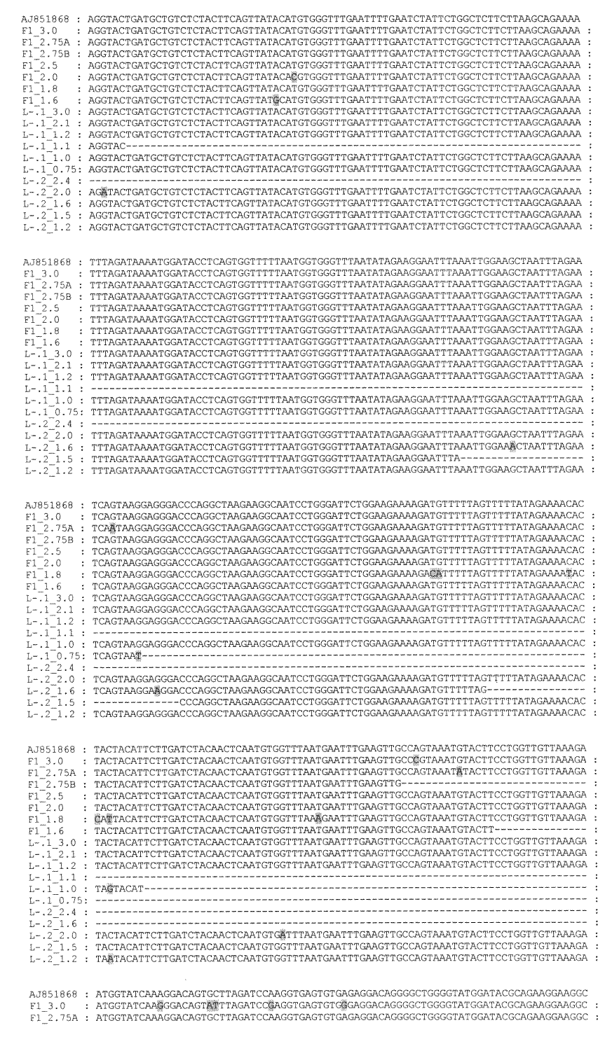

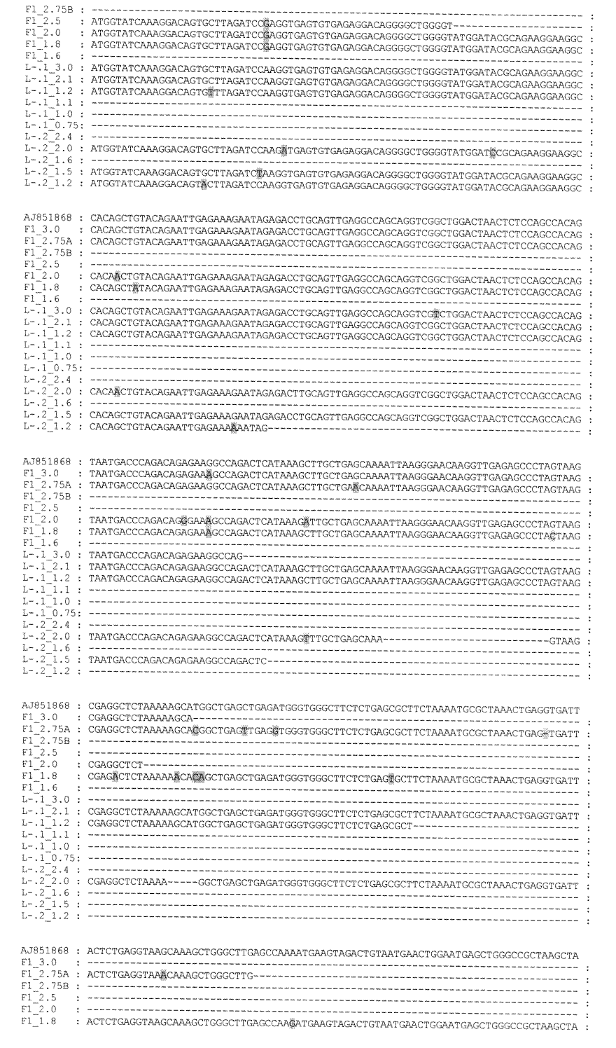

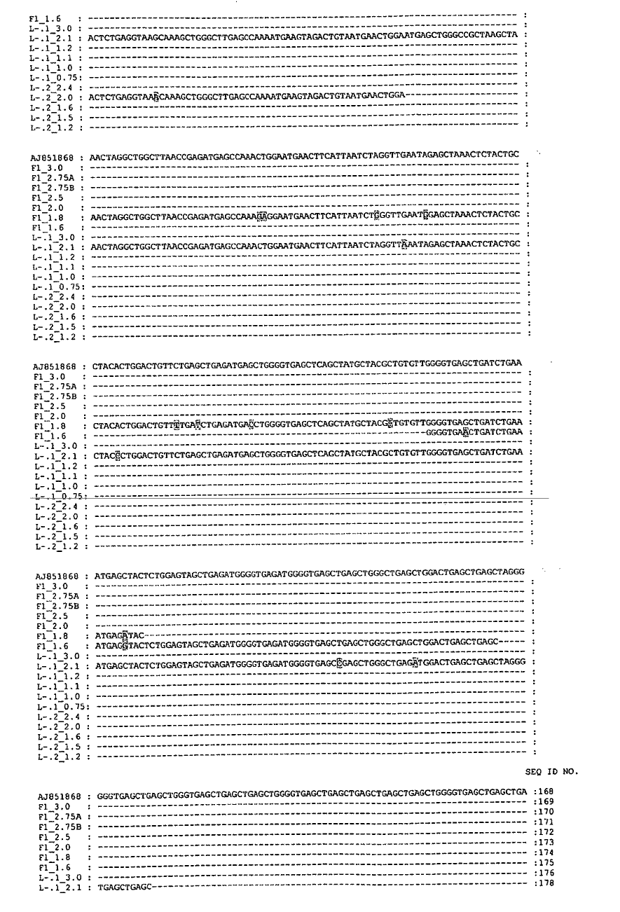

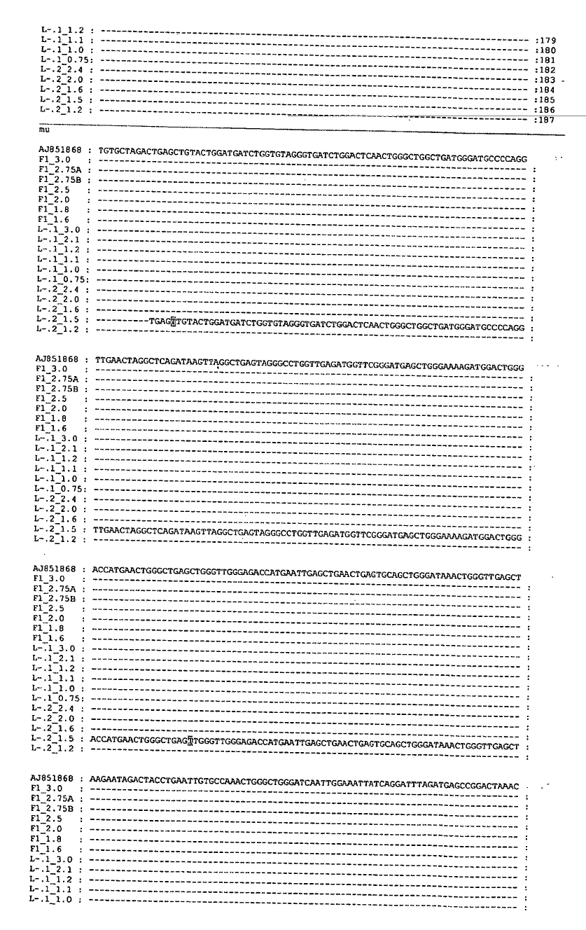

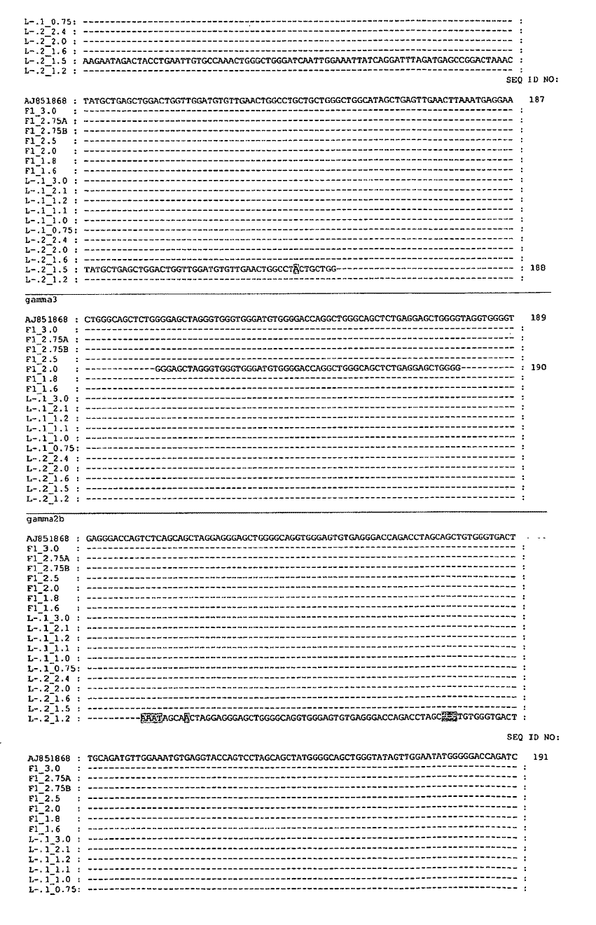

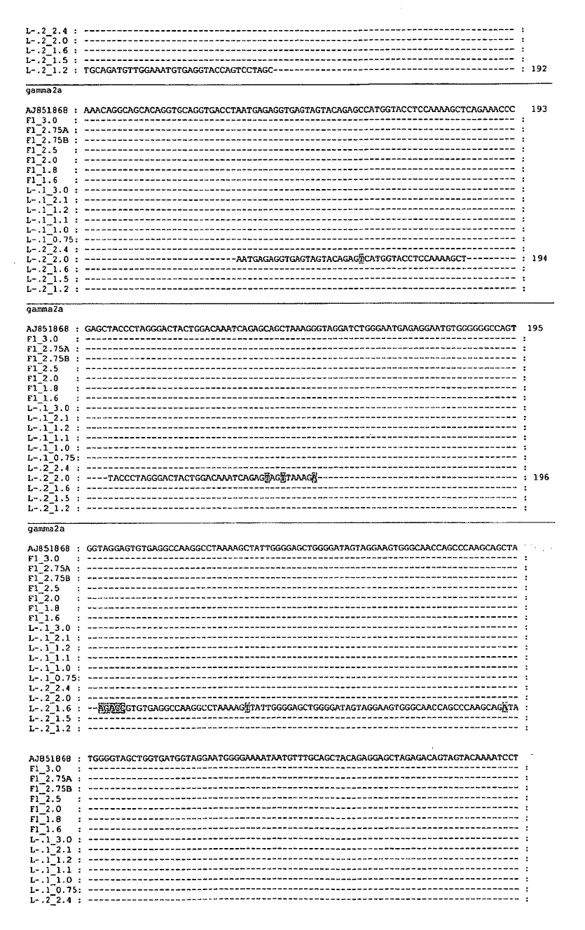

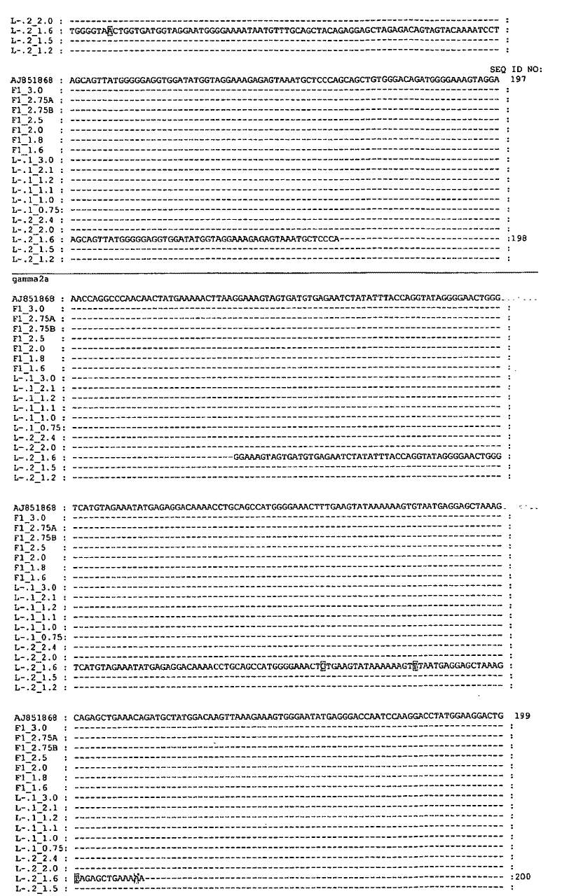

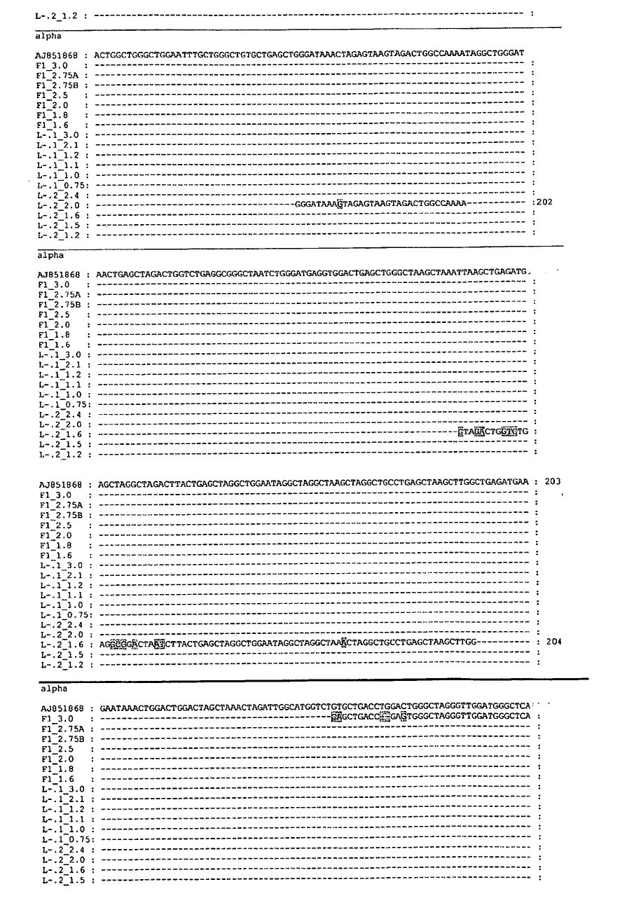

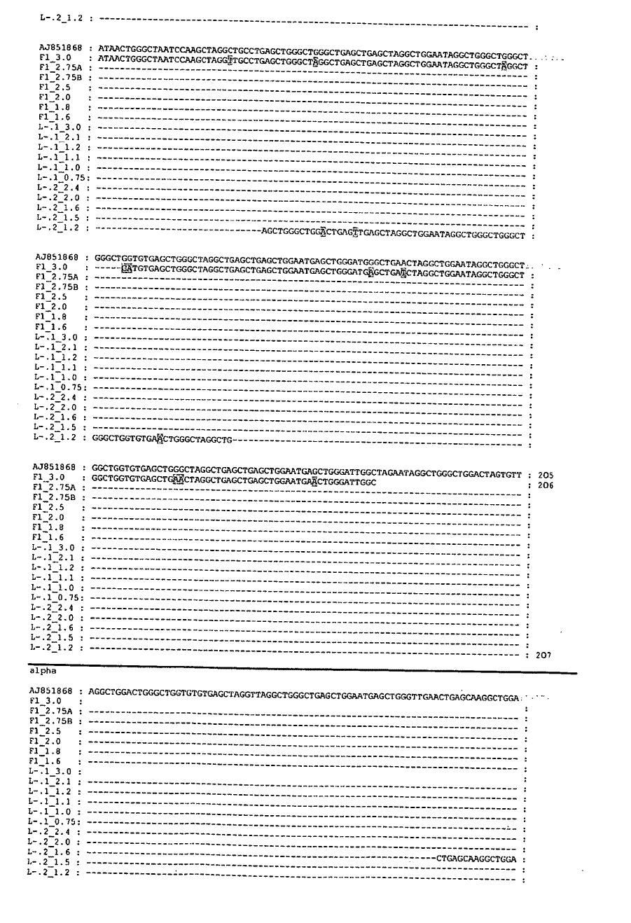

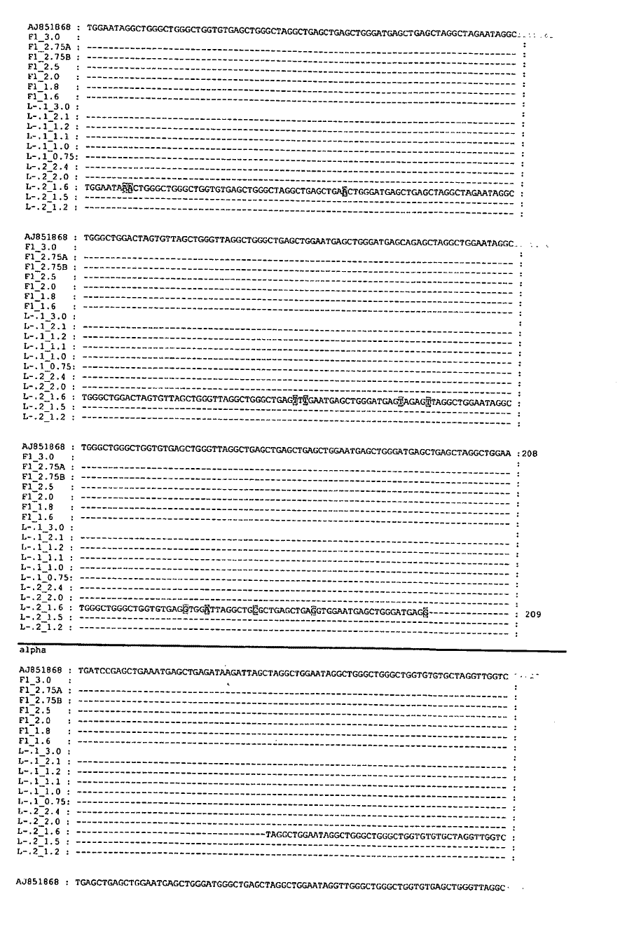

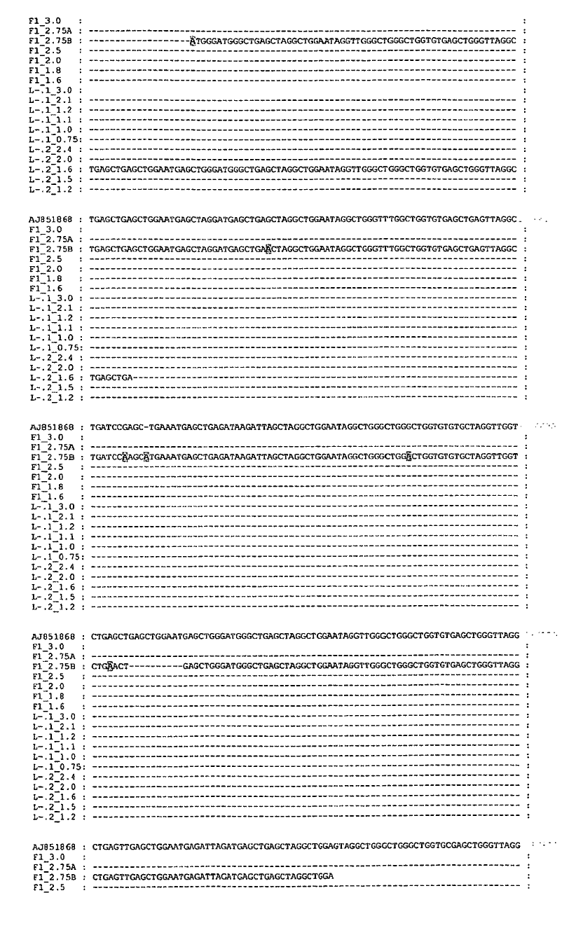

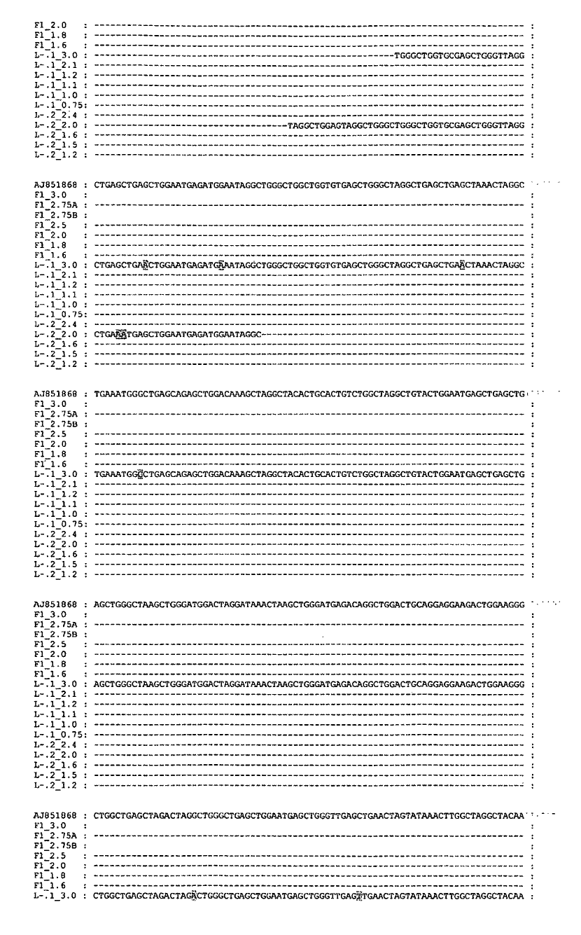

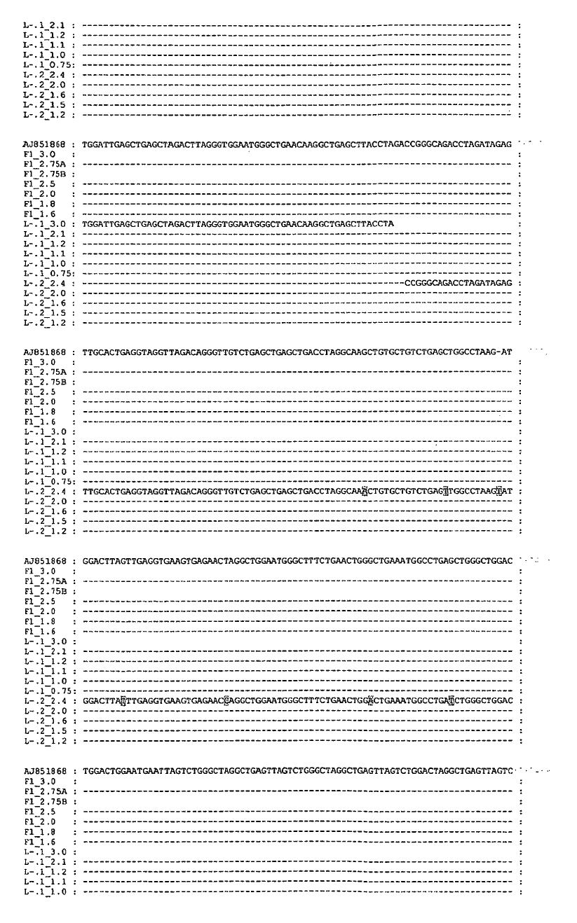

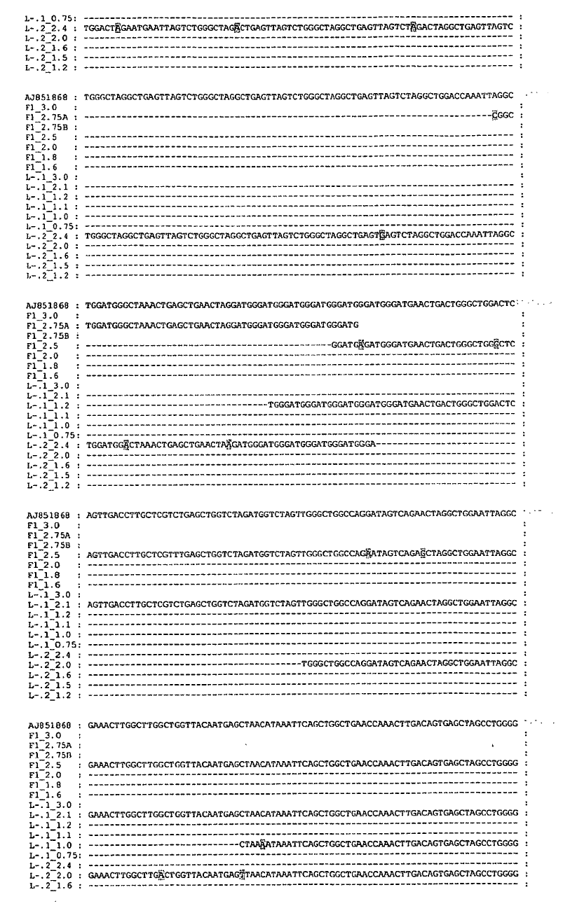

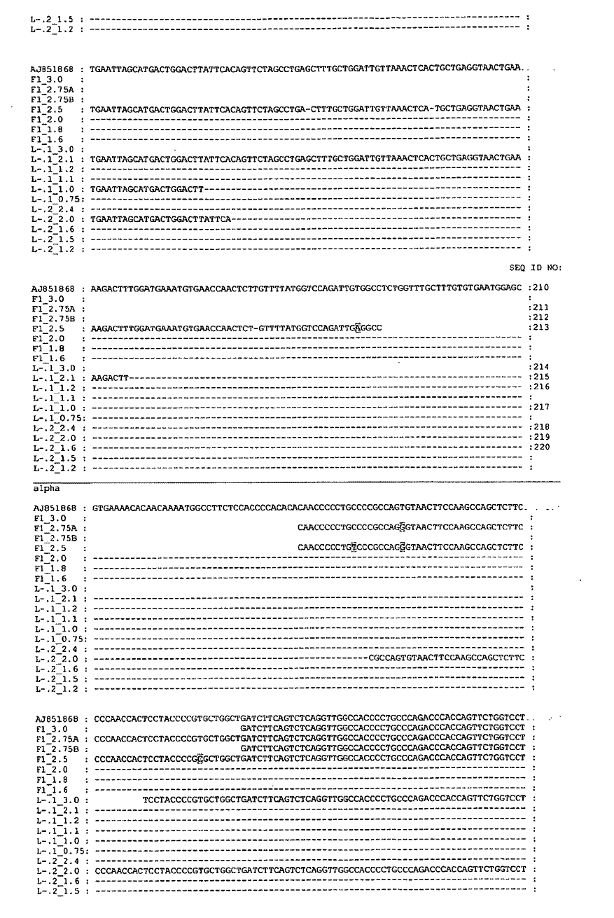

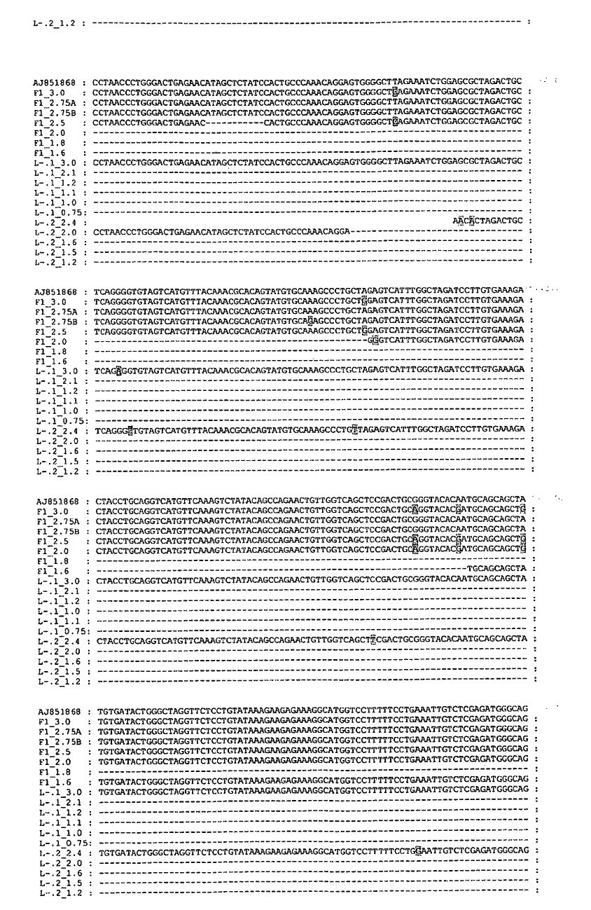

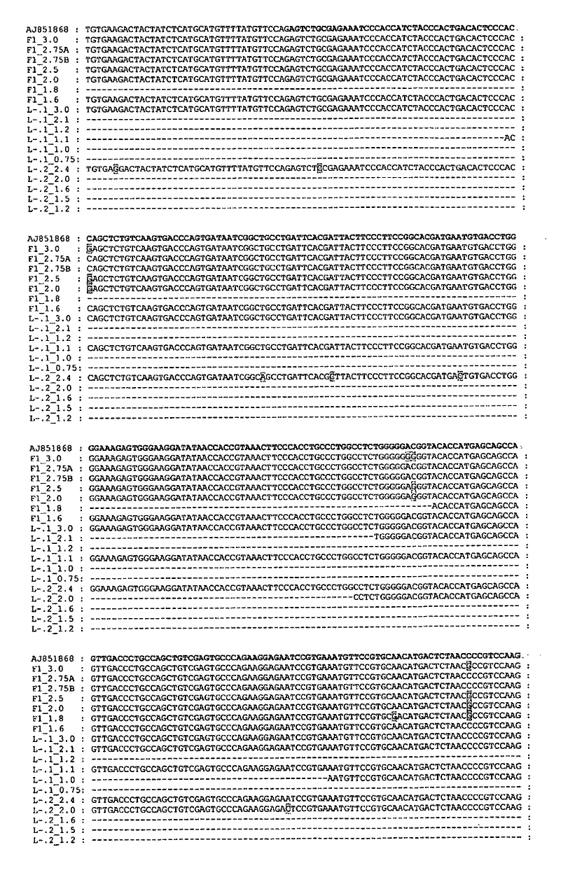

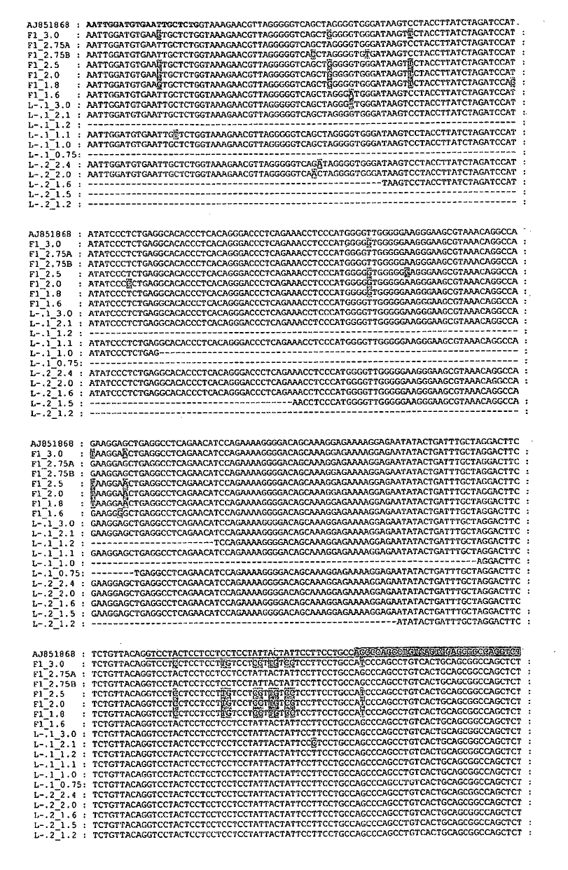

FIG. 18 shows long-range PCR gel electrophoresis and structure identifying diverse class-switch mediated deletions removing Cα1. DNA was prepared from sorted syndecan⁺ spleen cells (86) from two L^{-/-} mice and one normal animal. (A) The layout of the rearranged and switched (sp/(γ/)α) J_{H}4 to Ca region, 3-5 kb, is shown with external (black) and nested (shaded) primers indicated by arrows. (B) Gel separation of a nested PCR reaction, from 3′Eμ to Cα2L2 internal, which followed the initial PCR amplification from J_{H}4L to Cα2L1. (C) Cloning and sequencing of the PCR fragments indicated by thin lines identified complete and incomplete deletions of the Ca proximal switch region and the Cα1 exon (lightly shaded line/boxes). Cα2 is retained and the fragments of 3.0, 2.1, 1.2, 1.1, 1.0 and 0.75 kb correspond to the bands in the gel. Black lines/boxes indicate regions present in each fragment. Full sequencing information is provided in FIG. 22.

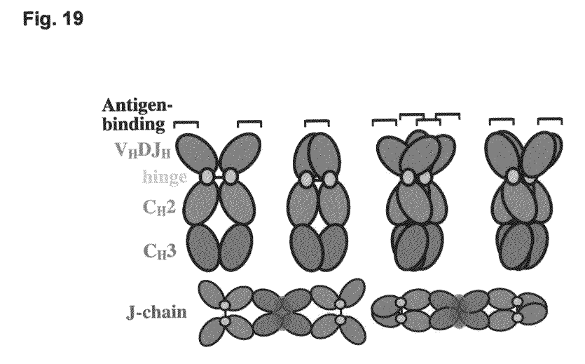

FIG. 19 is a diagram showing configurations of H-chain-only IgA. Based upon SDS-PAGE shown in FIG. 13C, H-chain-only IgA appears to be predominantly dimeric and to a lesser extent tetrameric. As a dimer, 2 binding sites may be generated or the 2 V_{H}-regions may associate to form one binding entity (as indicated). A similar organization may be possible when associating as a tetramer: with 4 or 2 binding sites. The potential association with the J chain (bottom) may separate the polarity of the binding sites to diametrically oppose each other.

FIG. 20 shows truncated αH-chains with mutational alterations identified by RT-PCR. V_{H} genes were identified from 5 mice. # Several independent clones with the same V_{H}DJ_{H} suggest iterative mutation. Mutations are underlined and P/N additions at V_{H} to D to J_{H}, C_{H}2 and C_{H}3 are indicated.

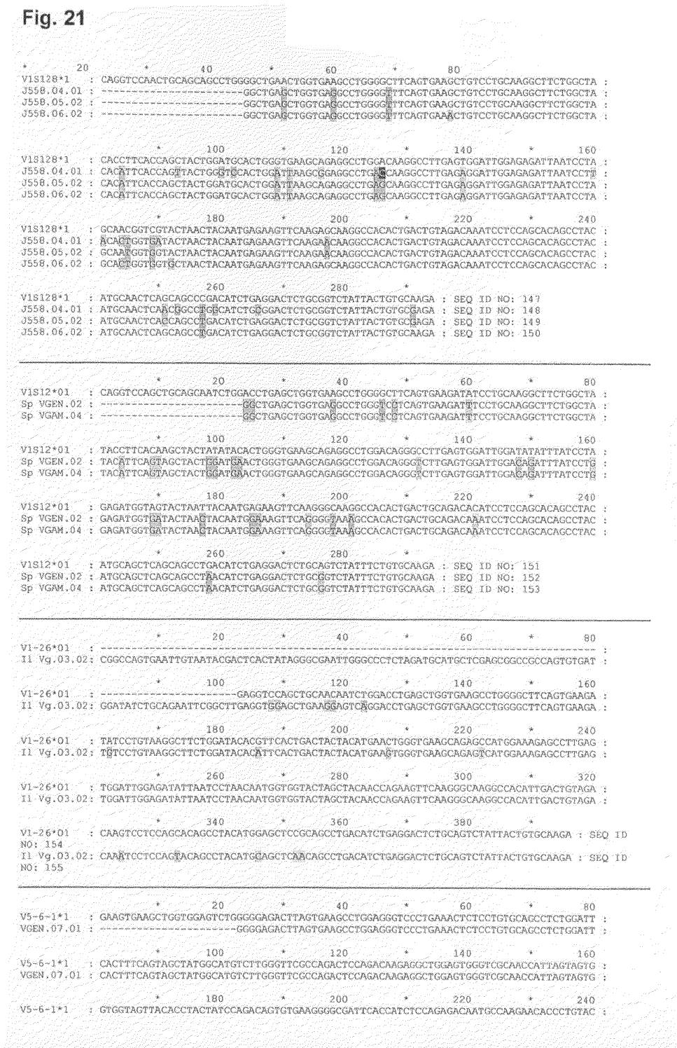

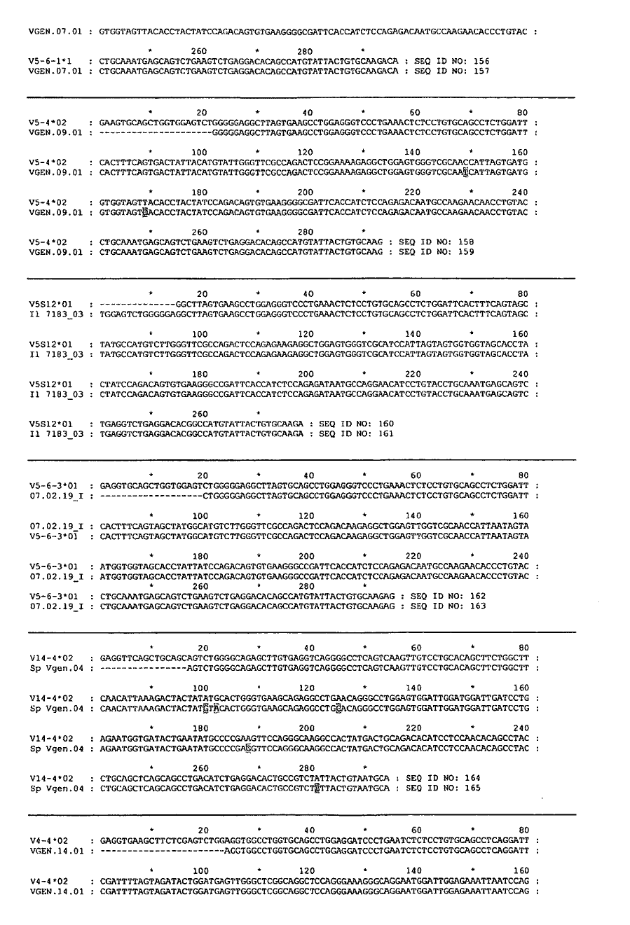

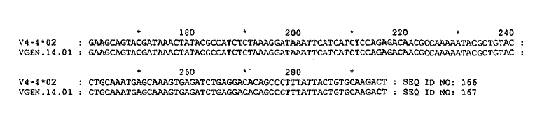

FIG. 21 shows RT-PCR sequences used for FIG. 20. V_{H} gene mutations not found in the data base are boxed.

FIG. 22 shows genomic DNA sequence information on which FIG. 18 is based. The region 3′Eμ includes switch sequences from μ, γ and α followed by Cα1 (bold) and Cα2 (yellow underlined [indicating the fused hinge region] and in pale blue).

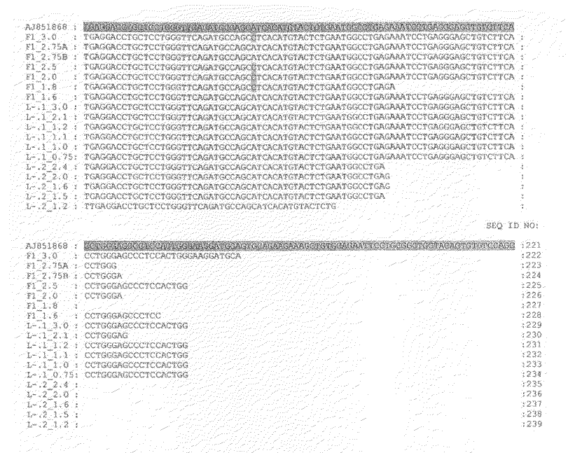

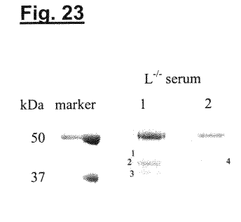

FIG. 23 shows serum IgA from two L^{-/-} mice purified by binding to anti-mouse IgA-conjugated sepharose, and size separated by SDS-PAGE. Mass-spectrometry, after trypsin digest of the bands indicated (1, 2, 3 and 4), identified a total of 11 different peptides (9 in mouse 1 and 10 in mouse 2) within the C_{H}2 and C_{H}3 exons of IgA; no fragments within C_{H}1 were found.

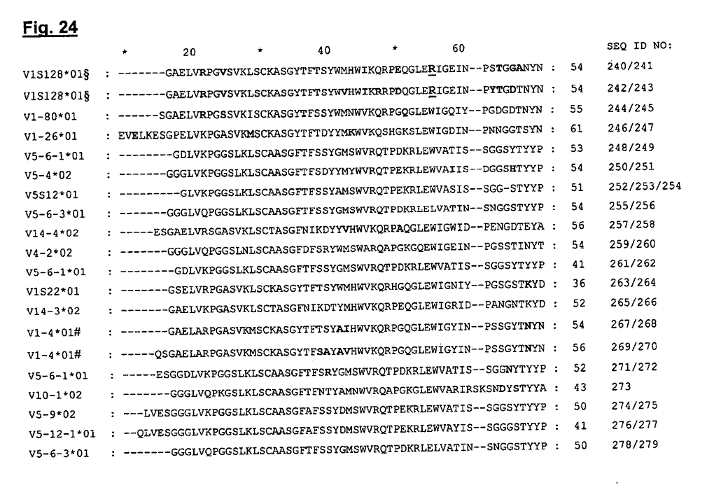

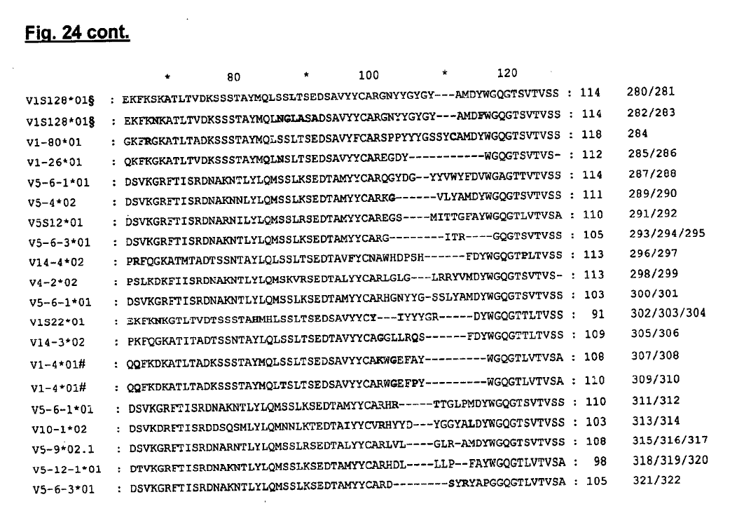

FIG. 24 shows the predicted amino acid sequence of the V region (V_{H}-D-J_{H}) with mutational alterations marked in bold. The V_{H}H hallmark, arginine in position 48 (Muyldermans et al., Trends Biochem. Soc. 26:230, 2001) in V1S128*01 (top), is underlined. Identical V_{H}-D-J_{H} joints (marked § and #) with different mutations are probably the product of clonal expansion.

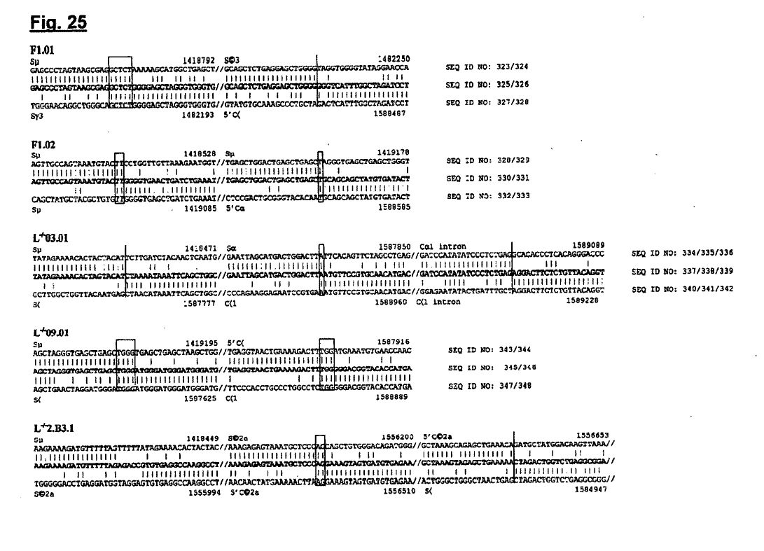

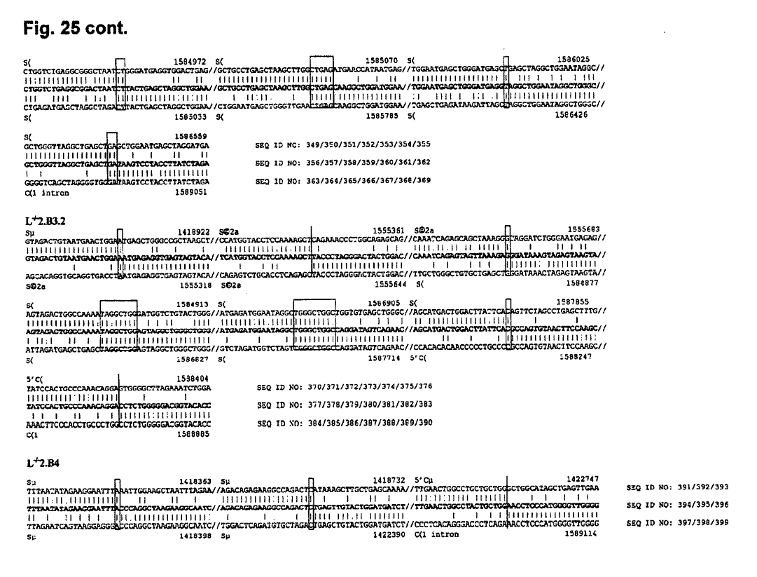

FIG. 25 shows alignment of normal mouse (F1) and L knock-out (L^{-/-}) mouse genomic sequences exhibiting multiple switch junctions. The GenBank™ accession number is AJ851868. Breakpoints are indicated by a vertical line or, if there is homology at a junction, the sequence is boxed. Switch regions were defined as the sequence 3′ of Eμ indicated in accession numbers J00442 (Sμ), D78343 (Sγ3), D78344 (Sγ2a and Sγ2b) and D11468 (Sα).

### EXAMPLE 1

In the following example we report that the absence of L-chain does not prevent serum antibody production in mice. Quite unexpectedly, we found antibodies in the serum of L-chain deficient mice without any further genetic manipulation other than functional silencing of the lambda and kappa L-chain loci. Diverse H-chain-only IgG without C_{H}1 is secreted despite compromised B cell development. We show that H-chain-only IgGs are produced from transcripts lacking the C_{H}1 exon, and we identify in some somatic cells different genomic deletions that can give rise to these transcripts. The results show that L-chain deficient animals are a useful tool for the production of H-chain-only antibodies such as therapeutic H-chain-only antibodies.

### 1.1 Materials and Methods

Mice. The derivation of Igκ and Igλ deficient (L^{-/-}, with Cκ disrupted by neo insertion and a ˜120 kb region from Cλ2 to Cλ1 removed by targeted integration and Cre-IoxP deletion), Cμ truncation (μNR, lacking Cμ1 and Cμ2 by targeted integration of neo), C deletion (CΔ, lacking a ˜200 kb region from Cμ to 3′ of Cα removed by targeted integration and Cre-IoxP deletion) and μMT mice has been described (7, 17, 45, 6). L^{-/-} and μNR animals were crossbred to homozygosity. Mice, 12-28 weeks old, were analyzed and compared with littermates or age-matched controls.

ELISA and immunization. Serum antibodies were analyzed as described (46) on Falcon plates coated with 10 μg/ml anti-mouse IgM, Igκ (Sigma) or IgG (Binding Site). Biotinylated detection antibodies were anti-mouse IgM (Sigma), IgG (Amersham), Igκ (Zymed) and Igλ (BD Pharmingen). To determine the antibody concentration purified IgG (DB3) was used (47). Immunisations were carried out with 100 μg OVA in CFA (s.c.) and subsequently 50 μg OVA in FA (i.p.), 30 and 14 days later, respectively. Ig secretion was identified by ELISA on plates coated with 10 μg/ml OVA (Sigma).

Western analysis. Serum Ig was incubated with anti-mouse Ig(μ, γ and α H-chain specific)-coupled Sepharose, separated on Ready-Gels (Bio-Rad) and transferred to nitrocellulose membranes as described (27). Filters were incubated with biotinylated anti-mouse Ig: μ-chain (Sigma), γ-chain (Amersham) and κ (Zymed) and λ (BD Pharmingen) L-chain specific. This was followed by incubation with streptavidin biotinylated HRP solution (Amersham) and visualization of bands using SuperSignal West Pico chemiluminescent substrate (Pierce, Illinois). Protein mol wt standards were supplied by Bio-Rad and Fermentas.

Flow cytometry. Bone marrow, spleen and peritoneal cell suspensions were prepared and multi-color analyses were carried out on a BD FACSCalibur. Cells were stained, in combination, with labeled anti-mouse Ig recognizing CD45R (B220) either PE- or APC- or BIO-conjugated, PE-conjugated anti-c-kit (CD117), BIO-conjugated anti-CD43, BIO-conjugated anti-CD25, FITC-conjugated anti-IgD, PE-conjugated anti-Igκ, FITC-conjugated anti-Igλ, PE-conjugated anti-CD5 (Ly-1), FITC-conjugated anti-CD79b (Igβ), FITC-conjugated anti-mouse CD21/35 (all from BD Pharmingen) and FITC-conjugated anti-IgM (Zymed). Reactions with BIO-conjugated antibodies were subsequently incubated with Tri-color-conjugated streptavidin (Caltag). CellQuest software (BD Biosciences) was used for the analysis.

For sorting on a BD FACSAria, spleen cells were stained with PE-conjugated anti-CD45R and, separately, FITC-conjugated anti-CD45R and BIO-conjugated anti-CD138 (syndecan-1) (BD Pharmingen) followed by incubation with PE-Cy5.5-conjugated streptavidin (eBioscience). For the analysis of cytoplasmic Ig, spleen cells stained for surface CD45R, were incubated with Fc-specific FITC-conjugated anti-IgG (Sigma) using a fix and perm cell permeabilization kit (Caltag). IgG positive cells were collected and viewed on a Zeiss confocal microscope (LSM 510 META) and images were obtained using Zeiss LSM 3.2 software.

RT-PCR analysis. RNA was isolated from tissue or sorted cells using Trizol (Gibco-BRL) and reverse-transcribed at 42° C. with Omniscript reverse transcriptase (Qiagen). PCR reactions with J_{H} and γC_{H}2^{a} primers were set up using KOD Hot Start DNA polymerase (Novagen), at a final MgSO₄ concentration of 0.8 mM. The cycling conditions were 94° C. for 2 min, 40 cycles of 94° C. for 15 sec, 58° C. for 30 sec, 72° C. for 15 sec, followed by 72° C. for 10 min. Amplification with V_{H} specific primers was as above, but with a 52° C. annealing temperature for Vgeneric, V3609 and VS107/J606. RT-PCR products were either sequenced directly or cloned by adding a 3′ A overhang and using a TA Cloning Kit (Invitrogen). β-actin PCR was performed as above but with an annealing temperature of 61° C. The J/hinge to γC_{H}2^{c} PCR was set up as above but with a touchdown program: 94° C. for 2 min, 21 cycles of 94° C. for 15 sec, 69° C. (−0.33° C./cycle) for 30 sec, 72° C. for 10 sec, followed by 25 cycles of 94° C. for 15 sec, 62° C. for 30 sec, 72° C. for 10 sec. For linear amplification of cDNA ends first-strand cDNA synthesis and primer extension was performed on 1 μg of total RNA as described (BD SMART mRNA Amplification Kit). Double stranded cDNA was purified using Wizard SV Gel PCR Clean-Up System (Promega) and linear amplification of the 5′ ends was carried out using KOD and nested γC_{H}2 primers (γC_{H}2^{b} and γC_{H}2^{a}) as described (48). The product from the fourth round of amplification was separated by agarose gel electrophoresis and bands between 700 and 1200 by were excised and purified using the Wizard kit. The purified fragments were cloned as above and sequenced. All primer sequences used are listed in Table 1 below. Agarose gels were run with size markers in kb (Hyperladder 1, Bioline) and/or by (100 by DNA ladder, Invitrogen).

TABLE 1
Primer sequences (all shown 5′-3′) for PCR

| | |
|---|---|
| Vgeneric | SADGTBCAGCTKMAGSAGTCWGG |
| V3609 | CARRTTAYTCWGAAASWGTCTGG |
| VS107/J606 | GARGTGMAGCTKGWDGARWCTGR |
| J558 | SAGGTYCARCTSCARCAGYCTGG |
| VGAM | CAGATCCAGTTSGTRCAGTCTGG |
| V7183/VH11 | GAMGTGMAGCTSKTGGAGWCTGG |
| J_{H}1 | CGGTCACCGTYTCCTCAG |
| J_{H}2 | GCACCASTCTCACAGTCTCCT |
| J_{H}3 | GGGACTCTGGTCACTGTCTCT |
| J_{H}4 | AACCTCAGTCACCGTCTCCTC |
| J/hinge | CACCGTCTCCTCAGAGCCC |
| γC_{H}2^{a} | TGTTGACCYTGCATTTGAAC |
| γC_{H}2^{b} | TTKGAGATGGTTYTCTCGATG |
| γC_{H}2^{c} | GTTGACCTTGCATTTGAACTCC |
| γC_{H}2^{d} | TTGGAGGGAAGATGAAGACGGATGG |
| γC_{H}2^{e} | TGTTGACCYTGCATTTGAACTCCTTGCC |
| β-actin2 | GATATCGCTGCGCTGGTC |
| β-actin4 | CTACGTACATGGCTGGGGTG |
| VDJ029 | CGGGGGGCTACGGCTACGTATGGG |
| J_{H}4long | GGAACCTCAGTCACCGTCTCCTCAG |
| γ2bhingelong | AGTGACTTACCTGGGCATTTGTGACACTC |
| γ2aC_{H}2long | AGGGCACTGACCACCCGGAG |
| 3′Eμ | GACCTCTCCGAAACCAGGCACCGC. |

Genomic DNA analysis. Genomic DNA was prepared as described (27) with the addition of linear acrylamide to aid the precipitation of small amounts of DNA from the sorted cells. Long PCR was carried out with Platinum PCR Supermix High Fidelity (Invitrogen). Reactions were set up with DNA from 1-2×10³ sorted cells (˜10 ng), equivalent amounts of ES cell or hybridoma DNA and 100 nM of each primer. The reactions with unsorted spleen DNA contained ˜100 ng DNA. An initial denaturating step of 94° C. for 1 min was followed by 94° C. for 15 sec and 68° C. for 15 min. A first round PCR of 20-36 cycles from VDJ or J_{H}4long to γC_{H}2^{e} was followed by a nested second round PCR of 15-30 cycles from 3′ Eμ to γC_{H}2^{d}, δ2aC_{H}2long or γ2bhingelong. Any bands obtained were cloned as described above and sequenced.

Mass-spectrometry. Coomassie-stained bands were destained, reduced, carbamidomethylated and digested overnight with 10 ng/μl Sequencing Grade Modified Trypsin (Promega) in 25 mM NH₄HCO₃ at 30° C. The resulting peptide mixtures were separated by reversed-phase liquid chromatography on a Vydac C18 column (0.1×100 mm, 5 μm particle size), with a gradient of 0-30% acetonitrile over 30 min, containing 0.1% formic acid, at a flow rate of 500 nL/min. The column was coupled to a nanospray ion source (Protana Engineering) fitted to a quadrupole-TOF mass spectrometer (Qstar Pulsar i; Applied Biosystems/MDS Sciex). The instrument was operated in information dependent acquisition mode, with an acquisition cycle consisting of a 0.5 sec TOF scan over the m/z range 350-1500 followed by 2×2 sec MS/MS scans (triggered by 2+ or 3+ ions), recorded over the m/z range 100-1700. Proteins were identified by database searching of the mass spectral data using Mascot software (Matrix Science).

RNA-FISH. Sorted cells were fixed on slides and analyzed by two-color RNA FISH. Methods for the analysis, with probes for FISH generated previously, have been described (ref 23 and refs therein). Images were visualized using Olympus BX40 and BX41 microscopes. Experiments were performed 2 to 4 times and at least 100 nuclei or 200 alleles were counted each time.

### 1.2 Results

IgG Expression without L-Chain

We initially aimed to investigate whether mechanisms for single H-chain Ig expression are naturally present in the mouse and are used if production of conventional antibodies is prevented. This was examined by using mice with silenced Igκ and Igλ L-chain loci (L^{-/-}) obtained by gene targeting (7). In the L^{-/-} mice all C_{L} genes are either disrupted (Cκ, Cλ₁) or removed (Cλ₂, Cλ₄ and Cλ₃) which prevents the production of functional L-chain (FIG. 1A). Although V_{L} to J_{L} rearrangement is retained and low levels of some truncated transcripts can be detected, no truncated L-chain products were identified in serum and cells. Unexpectedly, antibodies were found in these mice with serum IgG levels of at least 20 μg/ml and with some L^{-/-} animals reaching over 100 μg/ml (FIG. 1B). This was surprising as normal IgG cannot be secreted in the absence of L-chain and there is a block in the development of immature bone marrow B cells in these mice (7). The L^{-/-} mice were crossed with μNR mice (17), which express truncated IgM lacking Cμ exon 1 and 2, which prevents chaperone retention of the H-chain in the ER. We envisaged that this would allow μ transport to the cell surface and enable B cell differentiation to continue, resulting in higher IgG levels. As predicted, IgM without L-chain was secreted in μNRL^{-/-} mice whilst, in L^{-/-} mice, where no provision was made for the transport of H-chains to the cell surface, no IgM was detected. However, as shown in FIG. 1B, IgG levels appear to be unaffected by the dramatic increase in B cell numbers in the μNRL^{-/-} mice.

To establish unambiguously whether surface IgM expression is essential to drive H-chain IgG expression we crossed L^{-/-} mice with μMT animals, which carry a targeted disruption of the μ transmembrane exons (6). Serum analysis of heterozygous and homozygous littermates established that H-chain-only IgG secretion is only operative when the transmembrane configuration of Cμ is unaltered (FIG. 1 C). In L^{-/-}μMT^{-/-} mice no serum H-chain-only IgG was present whilst in L^{-/-}μMT^{+/-} mice serum IgG levels were maintained. This suggests that early B cell differentiation events are essential to produce H-chain antibody secreting cells.

Whereas B cell differentiation, resulting from the presence of truncated IgM did not increase the level of H-chain-only IgG produced by L^{-/-} mice the amounts could be considerably increased by a conventional immunization regime (FIG. 1 D). This procedure also revealed increased titers of OVA-specific IgG after several encounters with antigen.

### Size of Secreted Murine H-Chains

To determine the mol wt and assembly of these novel murine H-chain-only antibodies, Western blot analysis was performed on serum Ig separated under reducing and non-reducing conditions (FIG. 2). FIG. 2 A shows that γ H-chains (44-48 kD), but no μ or L-chain, could be detected in L^{-/-} serum. This new type of H-chain IgG is smaller than conventional IgG but comparable in size to dromedary IgG (18). H-chain-only IgG of the same reduced size is also produced in μNRL^{-/-} mice, in addition to H-chain-only IgM, which is of the predicted reduced size (17). Separation under non-reducing conditions revealed covalent linkage of two y H-chains with a combined mol wt of ˜92 kD, implying a homodimeric structure of H-chain-only IgG whereas H-chain-only IgM appears to be unlinked (FIG. 2 B). Detailed analysis of gel slices by mass-spectrometry, obtained after protein-A adsorption of serum protein and separation by SDS-PAGE, revealed IgG2b, IgG2a and IgG1 H-chain fragments from C_{H}2 and C_{H}3 exons but nothing from C_{H}1 (FIG. 2 C). In addition, sequences from 5 different V_{H} gene families were identified: V7183, VGAM3.8, J558, SM7 and J606.

### L-Chain Independent B Cell Development

Identification of substantial amounts of diverse H-chain-only Ig in the serum of mice lacking L-chains prompted extensive analysis of B cell differentiation events using flow cytometry (FIG. 3). Analysis of bone marrow cells from L^{-/-} and μNRL^{-/-} compared to normal and μNR mice, showed that developmental progression up to the pre B1 stage, identified by staining for B220 in combination with c-kit, CD43 or CD25, is largely sustained (FIG. 3 A). IgM expression without conventional L-chain is not maintained in L^{-/-} mice, whilst truncated IgM in μNRL^{-/-} mice reaches the cell surface but at decreased level compared to μNR mice. Similarly IgD is not, or very poorly, expressed on the cell surface without κ or λ L-chains. H-chain truncation in μNR mice leads to a substantial increase in CD5⁺ B220⁺ cells, identified as B1a lymphocytes (ref 17 and refs therein), which is not seen in L^{-/-} mice. Although the early stages of pre B cell development occur without L-chain, B cells expressing solely H-chain-only antibodies in L^{-/-} mice cannot be unambiguously identified by cell-surface staining. However, RT-PCR did yield a J/hinge to Cγ membrane sequence from B220⁺ spleen cells (not shown) but we have not yet cloned a complete product from V_{H} to the membrane exon lacking C_{H}1. In contrast μNRL^{-/-} mice retain cells expressing a BCR without L-chain, probably in association with Igβ.

The cells in μNRL^{-/-} mice that have acquired the expression of a H-chain-only BCR may overcome the block in conventional B cell differentiation and be released into the periphery as mature B cells. Proliferation of such cells may explain the distinct B220⁺CD21/35⁺ B cell population of splenic lymphocytes in L^{-/-} mice (FIG. 3 B), which may express IgM but little or no IgD. The small distinct population of B220⁺Igβ⁺ cells (0.8%) in L^{-/-} mice, which is much increased in μNRL^{-/-} mice (8.4%), may also suggest that mature cells can proliferate and maintain a conventional surface marker profile even without L-chain. Further analysis of peritoneal cells (FIG. 3 C) suggested an increase in larger or differently shaped cells not contained in the conventional lymphocyte gate (see www.flowjo.com and refs 19, 20) but evident when plotting forward scatter against side scatter to visualize size and shape distribution. Increases in cell size, albeit much less pronounced, are also seen in bone marrow and spleen cell stainings (data not shown). Interestingly, analysis of cells in the conventional lymphocyte gate showed that, in μNRL^{-/-} mice, the lack of L-chain does not appear to affect the generation of CD5⁺ peritoneal B cells, which are very low in L^{-/-} mice. A reason may be that μNRL^{-/-} mice, despite a lack of L-chain, are similar to μNR mice, which assemble a truncated surface receptor unresponsive to stimulation (17).

H-chain transcripts lacking C_{H}1 are generated in L^{-/-} and normal mice The production of γ H-chain transcripts in different tissues from L^{-/-} mice was assessed by RT-PCR using J_{H} to γC_{H}2 amplifications, which in normal animals produces a ˜650 by band in lymphoid tissue (FIG. 4). In L-chain deficient mice a prominent novel band of ˜350 by appears in bone marrow, spleen and lymph nodes, indicating that γ H-chain transcripts of reduced size are generated in these lymphoid organs (FIG. 4 A). The slight variations in product size are due to the length of the individual J segment and/or Cγ hinge exons used. All J_{H} segments, except J_{H}1, have been readily identified. J_{H}1 amplification did yield bands, but some cross reactivity occurs between the different J_{H} primers and all sequenced products have so far not identified this J segment (primers are listed in Table 1 above). V_{H} usage in the shortened γ H-chain transcripts from L^{-/-} mice was determined by RT-PCR with V_{H}-specific primers or linear amplification of cDNA ends followed by cloning and sequencing. The products identified were unusually spliced, linking V_{H}DJ_{H} to hinge or C_{H}2, and all lacked the C_{H}1 exon in the Cγ gene (Table 2). The V domains showed diverse rearrangements of V_{H}, D and J_{H} segments, including mutational alterations in V_{H} and non-encoded additions at the V_{H} to D and D to J_{H} junctions (Tables 3 and 4 and FIG. 7). The loss of C_{H}1 agrees with the lower mol wt H-chain protein found in serum and the absence of this sequence in mass spectroscopic analysis (see FIG. 2). In addition to the lower size H-chain band a full size product with C_{H}1 is usually amplified from lymphocyte-containing tissues of L^{-/-} mice (sequence data not shown).

In some J_{H} to γC_{H}2 amplifications the normal mouse spleen cDNA control gave a faint ˜350 by band (FIG. 4 A right, [symbol] indicated) which on sequencing was found to lack C_{H}1 (data not shown). This smaller band is frequently obscured due to amplification of an abundance of normal size products. However, the presence of this band implies that normal mice can generate transcripts, which could produce H-chain-only antibodies. To investigate whether H-chain transcripts lacking γC_{H}1 are regularly produced in normal mice we designed oligonucleotides that would only recognize splice products where a J_{H} segment joins a γ2a or γ2b hinge exon. Surprisingly, in normal mice ˜340 by J_{H}-hinge transcripts are readily found in the spleen and frequently, but not always, in bone marrow (FIG. 4 B). Sequence analyses revealed a predicted functional product without γC_{H}1 (data not shown).

TABLE 2
H-chain transcripts identified by RT-PCR from L^{-/-} spleen cell
populations obtained by RT-PCR and/or cloning

| | V_{H} | D | J_{H} | C_{H} |
|---|---|---|---|---|
| 028^{a} | VH10 | SP2.2 | J4 | γ2b(no C_{H}1) |
| 029 | 7183 | FL16.2 | J3 | γ2b(no C_{H}1) |
| 030 | J558 | FL16.1 | J4 | γ2a(no C_{H}1) |
| 129 | VH10 | SP2.2 | J4 | γ2b(no C_{H}1) |
| 132 | VGAM3.8 | FL16.1 | J2 | γ2b(no C_{H}1, no hinge) |
| 133 | 7183 | SP2.2 | J4 | γ3(no C_{H}1) |
| 135 | J606 | FL16.1 | J4 | γ2a(no C_{H}1) |
| 208 | SM7 | FL16.1 | J2 | γ2b(no C_{H}1) |
| 213 | J558 | ST4 | J3 | γ2a(no C_{H}1) |

^{a}Numbers refer to the full sequences in FIG. 7 and Table 4 with 028 to 129 from unsorted spleen cells and 132 to 213 from syn⁺ spleen cells.

TABLE 3
Junctional diversity of L^{-/-} V_{H} sequences^{a}

| | | | | | | |
|---|---|---|---|---|---|---|
| 3′V_{H} | N₁ | D | N₂ | J_{H} | HINGE | 5′CH₂ |

| | | | | | | | |
|---|---|---|---|---|---|---|---|
| 028^{b} | TGTGTGAGACA | | CTACTATGATTACGAC | GGG | TATGCTATGGACTACTGG // TCCTCAG | AGCCC // CCCAG | CTCCTAACCTC |
| 029^{c} | TGTGCAAGAG | CGCCGGG | CTACGGCTAC | GTA | TGG // TCTGTAG | AGCCC // CCCAG | CTCCTAACCTC |
| | | GGG | | | | | |
| 030 | TGTGC | CCGAAG | CGGT | TTTA | ACTGG // TCCTCAG | AGCCC // CCCAG | CACCTAACCTC |
| 129 | TGTGTGAGACA | T | TACTATGATTACG | GGGGG | TATGCTATGGACTACTGG // TCCTCAG | AGCCC // CCCAG | CTCCTAACCTC |
| 132 | TGTGCAAGA | AGGGGAT | TTACTACGGTGATA | GA | ACTTTGACTACTGG // TCCTCAG | | CTCCTAACCTC |
| | | | CCTAC | | | | |
| 133 | TGTGCAAGACA | TG | TCTACTTTGATTACG | GT | TATGCGACGGACTACTGG // TCCTCAG | AGCCT // CCCAC | CTGGTAACATC |
| 135 | TGTACCAGG | | GGAGGTA | AAGGA | ATGGACTACTGG // TCCTCAG | AGCCC // CCCAG | CACCTAACCTC |
| 208 | TGTAATGCA | GGG | GGTGGTAACTAC | GTGGGG | CTTTGACTACTGG // TCCTCAG | AGCCC // CCCAG | CTCCTAACCTC |
| | | | | GG | | | |
| 213 | TGTGCAAGA | AGGGGAG | CAGCTCGG | C | CTTACTGG // TCTGCAG | AGCCC // CCCAG | CACCTAACCTC |

^{a}Mutational differences not found in corresponding germline gene segments from 129, BALB/c or C57BL/6 mouse strains are underlined.
^{b}For clone details see Table 2.
^{c}Corresponding genomic sequence identified.

### Plasma Cells Produce H-Chain IgG

Flow cytometry using established separation parameters (e.g. scatter gating) did not reveal any obvious candidates or distinct B cell populations that showed enrichment for the truncated transcripts. So the lymphocyte gate was extended to include larger or differently shaped cells in the sorting process (20, 21) as antibody production and conceivably H-chain Ig secretion could be linked to an increase in cell size (FIG. 4 C-E). This we thought would clarify whether cells, normally excluded from the conventional lymphocyte gate, would produce a single size, or both a shorter and normal length, H-chain transcript. Gated spleen cells from L^{-/-} mice (P1) were separated into B220 dull large (P4) or average (P5) and B220^{int/+} large (P3) or average (P2) populations and analyzed by RT-PCR (FIG. 4 C). Diverse H-chain products of ˜350 by were obtained solely from the large B220 intermediate cell population P3, which on sequencing lacked γC_{H}1. Other populations, except small B220⁻ cells, produced the conventional ˜650 by band. The analysis suggested that only a distinct spleen cell population, large B220^{int/+} cells, produces H-chain-only Ig, which may be accompanied by a full-size product. Cytoplasmic staining (FIG. 4 D) showed that large B220^{int/+} cells from L^{-/-} mice (D2) are indeed IgG⁺. The small B220⁺ cells (D3=P2 in FIG. 4 C), lacking the indicative smaller H-chain band but producing full-length transcripts, may either contain reduced amounts or incorrectly folded δ H-chain products poorly recognized by anti-IgG.

To understand whether IgG⁺B cells from L^{-/-} mice bear the features of conventional antibody secretors and thus are the product of normal lymphocyte differentiation events we carried out stainings for syndecan (CD138), which identifies plasma cells, in combination with B220 (22). Syndecan⁺ cells (S3) in FIG. 4 E show a unique RT-PCR band characteristic for V_{H}DJ_{H}-H-C_{H}2-C_{H}3 products as confirmed by cloning and sequencing (Tables 3 and 4, FIG. 7).

TABLE 4
Accession number, source and list of mutational alterations.

| Id | Accession | Strain | V-region | FR1 | CDR1 | FR2 | CDR2 | FR3 |
|---|---|---|---|---|---|---|---|---|
| 028 | AC073561 | C57BL/6J | IGHV10-1*01 | a44 > c K15 > T | g83 > t S28 > M | a138 > g | g158 > c S53 > T | a201 > g |
| | | | (VH10) | | c84 > g S28 > M | g142 > c V48 > L | | a205 > c |
| | | | | | | | | a207 > g |
| | | | | | | | | c216 > a |
| | | | | | | | | g232 > c E78 > Q |
| | | | | | | | | a234 > g E78 > Q |
| | | | | | | | | g236 > c S79 > T |
| | | | | | | | | a283 > t M95 > L |
| 029 | AJ851868 | 129/Sv | IGHV5-6-3*01 | c10 > t | c87 > t | t140 > g L47 > W | t153 > c | t175 > g Y59 > D |
| | | | (7183) | | c93 > t | c149 > g T50 > S | | t177 > c Y59 > D |
| | | | | | | | | c181 > a P61 > T |
| | | | | | | | | c240 > t |
| | | | | | | | | c252 > g S84 > R |
| | | | | | | | | t278 > g M93 > R |
| | | | | | | | | g279 > a M93 > R |
| 030 | AC073939 | C57BL/6J | IGHV1-66*01 | | | | a155 > t Y52 > F | t234 > a |
| | | | (J558) | | | | | a258 > g |
| | | | | | | | | g276 > a |
| | | | | | | | | a284 > t Y95 > F |
| 129 | AC073561 | C57BL/6J | IGHV10-1*01 | | g83 > c S29 > T | | | a207 > g |
| | | | (VH10) | | | | | g232 > c E78 > Q |
| 133 | AJ851868 | 129/Sv | IGHV5-6-1*01 | | | | a166 > c S56 > R | g198 > a |
| | | | (7183) | | | | | |
| 135 | AJ972404 | 129/Sv | IGHV6-6*02 | | | | | |
| | | | (J606) | | | | | |
| 132 | AJ851868 | 129/Sv | IGHV9-3*02 | | | a104 > g N35 > S | c173 > g P58 > R | g189 > t E63 > D |
| | | | (VGAM3-8) | | | g118 > a A40 > T | | |
| 208 | AJ851868 | 129/Sv | IGHV14-4*02 | | t94 > c Y32 > H | | c173 > a T58 > N | |
| | | | (SM7) | | | | | |
| 213 | AC090843 | C57BL/6J | IGHV1-9*01 | c58 > a L20 > I | c89 > g T30 > S | | | a262 > t T88 > S |
| | | | (J558) | t60 > a L20 > I | g91 > a G31 > S | | | a277 > g I93 > V |

### Allelic Exclusion and V_{H} Gene Selection is Maintained

Encouraged by the diversity of the H-chain antibodies found, we investigated whether activation of the IgH locus and diversity of V_{H} gene usage is equally operative in L^{-/-} mice compared to normal animals (23, 24). To detect individual IgH alleles we used RNA-FISH with a probe, Iμ, which establishes locus activity (FIG. 5 A). Iμ is a non-coding RNA transcript originating from the IgH intronic enhancer, immediately downstream of the J_{H} genes. It is expressed throughout B cell development and is used as a marker of an actively transcribing allele. In B220⁺ CD25⁺ pre B2 cells from normal mice, a 40:60% ratio of detection of Iμ transcripts from one or both IgH alleles, respectively, is observed following V_{H}DJ_{H} recombination (23). The 40% of cells with single Iμ signal represents the proportion of cells in which productive V_{H}DJ_{H} recombination has silenced the second, DJ_{H} rearranged allele by allelic exclusion, resulting in loss of Iμ transcription. The 60% of cells with Iμ signals on both alleles represents cells in which non-productive V_{H}DJ_{H} rearrangement on the first allele is followed by productive rearrangement on the second allele, and transcription of both types of V_{H}DJ_{H} rearranged allele. If allelic exclusion were impaired, the ratio would be expected to change to include more cells with double signals. However, in B220⁺ CD25⁺ pre B2 cells from L^{-/-} mice, similar ratios of single to double Iμ signals were observed (FIG. 5 B), suggesting that allelic exclusion of the IgH locus is maintained in these mice. In addition, detection of proportions of V_{H}DJ_{H} rearranged transcripts corresponding to the J558 (FIG. 5 C) or the 7183 (FIG. 5 D) V_{H} gene families on individual alleles were very similar between normal and L^{-/-} mice, indicating that a normal, diverse range of V_{H} genes is utilized.

### Acquired Genomic Alterations of C_{H}1 Accomplish H-Chain-Only Expression

RT-PCR and sequence analysis of smaller size H-chain bands identified a lack of γC_{H}1 or, in a few cases, both the γC_{H}1 and γhinge exons (Table 3). One way to identify mutations leading to H-chain-only antibodies is the derivation of hybridomas. Another approach of gaining access to cells expressing IgG is by sorting for syndecan positive cells. In order to enrich this starting material with DNA from cells expressing IgG, we set up an assay for amplifying switched γ regions. A long range PCR with a J_{H} primer and a γC_{H}2 primer, followed by a second nested reaction from 3′Eμ to γC_{H}2^{d} (FIGS. 6A and B) gave rise to bands whose size was consistent with that of switched γ regions. These switched γ regions could be amplified from normal or L-chain deficient DNA from sorted syndecan⁺ spleen cells but not from (germline) ES cell DNA. Cloning and sequencing of nested PCR products identified conventional exon and intron sequences regarded as functional (data not shown) and shorter sequences with deletions in and around γC_{H}1 (FIG. 6 C and FIG. 7, Table 4).

To establish unambiguously whether transcripts that lack C_{H}1 are the result of genomic deletions we derived forward primers from their D-J_{H} junction sequence. Successful amplification and cloning from a rearranged V_{H} of the 7183 family identified a large deletion removing the μ/γ switch region and C_{H}1 of Cγ2b concluding 107 nucleotides 5′ of the hinge exon (clone 029, Tables 3 and 4, FIG. 6 and FIG. 7). As the deletions that render γC_{H}1 dysfunctional remove a large part of the adjacent switch region it is possible that the DNA lesions occur during switch-recombination (25), leading to alterations in Cγ that permit H-chain-only antibody expression.

### 1.3 Discussion

In L-chain deficient mice, B cell development is arrested at the pre B2 to immature B cell stage in the bone marrow (7). At this transition stage, IgM, comprising a μ H-chain covalently linked with a κ or λ L-chain in dimeric configuration, should be expressed on the cell surface associated with the co-receptor chains Igα/β. Developmental progression of a compromised BCR lacking any of these chains is normally blocked (6-8, 26), so the finding of IgG in the serum of L-chain deficient mice came as a surprise. Analysis by Western blot and mass spectrometry indicated that these proteins were lacking the C_{H}1 domain. This was confirmed by RT-PCR, which identified short γ transcripts lacking C_{H}1 (or in some instances C_{H}1 and hinge) in the lymphoid organs of L-chain deficient mice. These findings are in agreement with reports for transgenic mice that express H-chain only Ig, where the loss of C_{H}1 appears to be essential (27-29). The shorter nascent-translated H-chain cannot form a complex with the H-chain binding protein as it lacks the association sites in C_{H}1 (15, 30). This would result in unhindered transport through the ER allowing surface deposition, as well as H-chain secretion. The stability of such H-chain-only Ig is remarkable and it can be argued that the lack of C_{H}1 and the loss of chaperone association may prevent degradation of a basically incomplete Ig. Sorting experiments indicated that the main sources of short transcripts were large, syndecan positive cells, i.e. plasma cells, and thus the product of normal lymphocyte differentiation, while conventional transcripts were abundant in a B220^{high} cell population. However, these results raised the question of how the protein deletions occurred and how antibody-producing cells could be generated in the absence of noticeable BCR expressing B cells.

Different mechanisms can lead to exon removal such as alternative splicing, splice-site mutations or exon deletions; all of which have been found for Ig genes (31, 32). If expression of H-chain IgG were controlled at the transcriptional stage, for example by selection of splice products leading to polypeptides which could be released from the cell, then the rearranged H-chain gene should be unaltered. To look for the existence of somatic mutations, we sorted syndecan positive cells, which are enriched for cells producing transcripts lacking C_{H}1, and extracted the DNA. Long range DNA-PCR analyses using 5′ primers in the J_{H} and Eμ region and reverse primers in the γC_{H}2 exon ensured that only switched γ genes were amplified. With this approach, we were able to clone three different genomic Cγ deletions where most or all of C_{H}1 and the μ/γ switch-region were removed, but the hinge exon and Eμ intron enhancer downstream of J_{H}, were left intact. With these modifications transcription levels and splicing from a rearranged V_{H}DJ_{H} to the hinge exon should be maintained, producing a truncated protein. A putative mechanism for C_{H}1 deletion suggested by our sequence data is error during the class-switch process. The switch-region upstream of each C_{H} gene is highly repetitive, several kb in length and accommodates repairs to DNA lesions, such as double strand breaks. The recombination itself, which removes Cμ and juxtaposes the rearranged V_{H}DJ_{H} close to a downstream C_{H} gene, occurs between non-homologous sequences without any consensus motif defining precisely the donor and acceptor breakpoints (33). It is possible that imprecise switching removes all or part of C_{H}1, which would allow Ig surface expression.

However, switching (and presumably faulty switching) occurs from mature IgM-expressing lymphocytes, which are difficult to identify in the spleen of L^{-/-} mice, occurring as a small population of B cells expressing high levels of B220. It is possible that failing to become a mature IgM-expressing B cell initiates early class-switching which may explain why serum IgM is absent in L^{-/-} mice and camelids do not appear to produce H-chain-only IgM or V_{H}HDJ_{H}-Cμ transcripts (34, 35). This possibility is strengthened by the fact that we can identify in the spleen full-length y transcripts that are much more abundant and diverse than short transcripts. Presumably, the switch from μ to γ occurs in a large number of cells, but in most cases, this does not allow production of a H-chain that can be transported to the cell surface without L-chain. Only when faulty switching gives rise to DNA sequences encoding transcripts lacking C_{H}1 would the B cell be selected for survival. The absence of H-chain IgG in L^{-/-}μMT^{-/-} mice suggests that a pre-BCR dependent proliferative stage is required; probably to produce the number of cells required to obtain these specific aberrant switching events. A knock-in gene encoding a mutant μ chain (μNR, ref 17) was introduced into the genome of L^{-/-} mice, which resulted in expression of a truncated μ H-chain on the cell surface in the absence of L-chain. This ensured cell survival but did not result in an increased IgG level, which could be seen as puzzling, as μNR mice expressing L-chain have a normal, high level of IgG. However, the pre-BCR dependent proliferative stage is slightly impaired in the μNR mice. Also, in μNR and μNRL^{-/-} mice, the expression of truncated μ significantly increases the number of a particular CD5⁺ lymphocyte subset, (CD5⁺ B1a cells), which rarely switch (36, 37). In addition to the deletion of the C_{H}1 region, we have identified C_{H}1 point mutations in some of the switched γ regions from L-chain deficient mice. However, none of them corresponds to a typical splice site mutation so it is not clear if these changes affect splicing. Further analysis is required to determine whether they cause exon skipping by altering exon recognition by cis-elements involved in the splicing process (38). If this is the case L-chain deficient mice might provide useful information on the mechanism controlling splice site usage.

Evidence that secretion of H-chain-only Ig in L-chain deficient mice is antigen-dependent, comes from the increased titers of specific antibody after immunization. Also several functional V_{H} sequences were found, which harbored mutations that can be attributed to somatic hypermutation (39). We investigated whether specific alterations compensate for the lack of L-chain association, as found in adapted camelid V_{H}H exons (34). From the alignment of V_{H}H sequences with the V regions of mouse H-chain antibodies it was found that this was not the case. None of the hallmark amino acids found in V_{H} to V_{H}H substitutions in framework 2 (Val37Phe, Gly44Glu, Leu45Arg and Trp47Gly) (40, 41) were seen, and in one case the reverse was found with an Arg to Leu change at position 45 (Kabat numbering). Some camelid H-chain Igs bear a long CDR3 and it has been suggested that CDR3s encompassing a more extensive D segment and/or substantial N-sequence additions may be an advantage to compensate for the smaller antigen-binding area of H-chain Ig compared to the conventional H-L Ig (34, 35, 40, 41). A longer CDR3 was not found with the mouse H-chain antibodies, but it should be noted that antigen-specific dromedary H-chain antibodies with shorter CDR3 (7aa) have also been identified (42).

Expression of H-chain-only IgG in L^{-/-} mice appears to differ from human HCD. HCD are monoclonal B cell lymphoproliferations secreting mutant H-chain not associated with L-chain. It has been hypothesized that these proliferations are caused by expansion of cells that express altered H-chains because they have previously lost the ability to produce L-chains (although there are cases where free L-chains are produced by tumor cells) (43). The results we have obtained show that the absence of L-chains leads to selection of cells producing mutant H-chain lacking C_{H}1, when normal competing B cells are absent. However, unlike in HCD, it appears from the Western blot analysis and the sequence data from L′^{-/-} mice that gross alterations in the V_{H} regions are not present in the majority of the cells. In addition, in L-chain deficient mice we have not observed the lymphoproliferations which occur in HCD.

The modifications observed in L-chain deficient mice produce a domain configuration comparable to that which has been identified in both camelids and cartilaginous fish; representing in the former a relatively recent adaptation (13) and in the latter a possible remnant of the primordial antibody structure that preceded the heterodimeric association of H- and L-chains (11). In lower vertebrates H-chain dimers have been recognized that lack a classical C_{H}1 domain, important to provide the cysteine residue that forms the disulphide linkage with the L-chain (11, 12). The evolution of Ig domains, their multiplication and diversification to permit functional interaction, vividly illustrates the ongoing selective pressure on antibody genes. Specific alterations, in the case of the L^{-/-} mice, the removal of C_{H}1, prevented Bip association without affecting H-chain dimerisation, an essential requirement to secure the antibody structure for immune protection (44).

In conclusion, Example 1 shows that in mouse B cells the removal of C_{H}1 permits cellular release of fully functional IgG antibodies without L-chain and development of a diverse H-chain-only antibody repertoire. Mouse V_{H} genes can be expressed as H-chain antibodies without acquiring V_{H}H specific changes and maintain their inherited sequence characteristics and lengths (41). A B cell repertoire with somatic hypermutation would be of great importance for the production of H-chain-only monoclonal antibodies in mice. Difficulties with generating hybridomas from L-chain deficient mice using whole organs (e.g. spleen) may be due to the small numbers of activated lymphoblasts present. This should be overcome for example by increasing the cell population of H-chain-only Ig producing progenitors. Since somatic alterations leading to C_{H}1 deletion, due to its very low frequency, is a strong limiting step in H-chain-only IgG production, deleting a γ C_{H}1 exon in the germline of L-chain deficient mice allows H-chain-only monoclonal antibodies with defined specificities to be readily produced.

### EXAMPLE 2

In this example, we show that IgM lacking the BiP-binding domain is displayed on the cell surface and elicits a signal that allows developmental progression even without the presence of L-chain. The results are reminiscent of single chain Ig secretion in camelids where developmental processes leading to the generation of fully functional H-chain-only antibodies are not understood. Furthermore, in the mouse the largest secondary lymphoid organ, the spleen, is not required for H-chain-only Ig expression and the CD5 survival signal may be obsolete for cells expressing truncated IgM.

As noted above, classical antibodies consist of multiple units of paired H- and L-chains and are produced by all jawed vertebrates studied to date. In addition, Tylopoda or camelids (camels, dromedaries and llamas) secrete dimeric H-chain-only IgG (9) similar to single chain Ig also found in primitive cartilaginous fish (11, 12). Homodimeric H-chain-only antibodies in camelids, as well as in lower vertebrates, either lack the C_{H}1 or a C_{H}1-type domain, respectively, which normally provides the disulphide linkage with the L-chain (11, 12, 49). Both antibody-types are produced by DNA rearrangement of the IgH locus, where D (diversity), J (joining) and V region gene segments are recombined, initiating B cell development in B lymphocyte progenitor cells (reviewed in 4, 50). Juxtaposition of V_{H}-D-J_{H} is followed by expression of a μ H-chain, which associates with the surrogate L-chain consisting of VpreB and λ5 and the co-receptor polypeptides Igα and Igβ to form the preB cell receptor (preBCR). After expansion of the preB cell pool, a preBCR negative stage occurs where μ H-chain is intracellular and surrogate L-chain is not detected (51). Production of κ or λ L-chain upon V_{L} to J_{L} rearrangement allows surface IgM expression as part of the BCR; a requirement so that a sizable number of cells can colonize the secondary lymphoid organs, such as spleen or lymph nodes (7). Further differentiation to produce class-switched isotypes is performed by replacing Cμ with a 3′ C_{H} gene, for example Cγ or Cα, in the class switch recombination process (52). The amino-terminal signal sequence of nascent H- and L-chains permits their shuttle to the lumen of the ER, which is accompanied by glycosylation and BCR assembly. Upon quality control of this reaction, disulphide linked chains are transported to the Golgi complex for further processing involving carbohydrate additions, which is followed by packaging into vesicles transported to the plasma membrane for release (53, 54). Analysis of IgM and IgG mutants have established that H-chain-binding protein (BiP, also termed GRP78) associates with the C_{H}1 domain of newly-synthesized Ig H-chains preventing their cellular release unless BiP is replaced by L-chain (55). However, H-chains not associated with BiP can be exported with or without L-chain; thus the lack of C_{H}1 in camelid H-chain IgG secures secretion. In humans the absence of V_{H} or C_{H}1 allows secretion without L-chain in rare B cell malignancies known as Heavy Chain Disease (HCD) (reviewed in 56). Also, in transgenic mice truncated H-chains can be released from the cell, not necessarily leading to malignant growth (17, 27, 57). Murine preB cells with full length μ H-chains on the cell surface without associated surrogate or conventional L-chain have also been described (58, 59, 60). More recently, the ability for some B cells to display H-chains on their surface in the absence of L-chains has been reported (61).

Our approach to understanding H-chain-only antibody expression and the importance of the Ig L-chain focused on the analysis of modified mice obtained by gene targeting and transgenesis. In Example 1 above we revealed that H-chain-only IgG can be produced naturally by removal of all or part of the C_{H}1 exon of a Cγ gene. Here we show that expression of a L-chain deficient BCR occurs in a high number of cells when stable interaction of the H-chain with BiP is prevented. The occurrence of B-1a lymphocytes, in mice expressing truncated IgM without L-chain, is independent of CD5, the cell surface receptor characterizing this B cell subset. Aborted spleen development in Hox11 knock-out (spleenless) animals revealed that asplenia with its missing B cell lineages does not prohibit single-chain antibody secretion, indicative of B1 cell independent expression.

2.1 Materials and methods

Mice. The derivation of Cμ truncation (μNR), ΔVμ transgenic, Igκ and Igλ deficient (L^{-/-}), CD5 deficient and Hox11 deficient mice has been described previously (7, 17, 62, 63, 64). Animals were crossbred to homozygosity to obtain the following new combination of features: μNR L^{-/-}, μNR CD5^{-/-}, μNR Hox11^{-/-}, Hox11^{-/-} L^{-/-} and ΔVμ L^{-/-}. Mice, 3-6 months old, were analyzed and compared with littermates or age-matched controls.

Flow Cytometry. Cell suspensions were prepared from bone marrow, spleen and peritoneal cells and multi-color analyses were carried out on a BD FACSCalibur. For the analysis cells were stained, in combination, with anti-mouse antibodies recognizing CD45R (B220) conjugated to either Phycoerythrin (PE), allophycocyanin (APC) or Biotin (BIO), PE-conjugated anti-c-kit (CD117), BIO-conjugated anti-CD43, BIO-conjugated anti-CD25, PE-conjugated anti-Igκ, FITC-conjugated anti-Igλ, PE-conjugated anti-CD5 (Ly-1), FITC-conjugated anti-CD79b (Igβ), FITC-conjugated anti-CD21/35 (all from BD Pharmingen), FITC-conjugated anti-IgM (Zymed) and FITC-conjugated anti-human IgM (Nordic). Reactions with BIO-conjugated antibodies were subsequently incubated with Tri-color-conjugated streptavidin (Caltag). CellQuest software (BD Biosciences) was used for the analysis.

ELISA. Serum antibodies were analyzed as described (Zou et al., 1995) on Falcon plates coated with 10 μg/ml anti-mouse IgM, Igκ (Sigma), Igλ (BD Pharmingen) or IgG (Binding Site) or anti-human IgM (Sigma). Biotinylated detection antibodies were anti-mouse IgM (Sigma), IgG (Amersham), Igκ (Zymed) and Igλ (BD Pharmingen) and anti-human IgM (Sigma). Standard deviation of the serum antibody titer was calculated from at least 5 age-matched mice except ΔVμ control where serum from 3 mice confirmed previous observations.

### 2.2 Results

### Genetically Altered Mice for the Analysis of Chaperone-Independent IgH Expression in B Cell Development

Antibodies without L-chain can be released from the cell as dimerised H-chains in H-chain-only Ig lacking C_{H}1 or as monomers, dimers or polymers in HCD. In both malignant and healthy expression of H-chain-only Ig, no full-length polypeptides are produced (49, 65). Alterations, such as the removal of the first C region exon in Cμ or Cγ are responsible for permitting BiP(chaperone)-independent cellular transport and discharge of H-chains without initiating an unfolded protein response (UPR), which normally leads to degradation of incomplete polypeptides (21, 55). Mice that do not express antibody L-chains generate a wide range of differently rearranged IgG H-chains, which have many of the attributes of H-chain-only IgG found in camelids (see Example 1 above). We wanted to gain information as to how BiP-independent H-chain release from the cell affects developmental processes. Mice carrying different combinations of transgenic and knockout alterations affecting lymphocyte differentiation events were analyzed by comparing cell surface staining using flow cytometry.

The alterations to the Ig loci (μ, κ and λ), the lymphocyte cell-surface marker (CD5) and the homeobox gene responsible for spleen development (Hox11) are depicted in FIG. 8. In μNR mice truncated IgM without C_{H}1 and C_{H}2 is produced (17), whilst ΔVμ is a transgenic line in which human Cμ expression is driven by a V_{H}-leader sequence without V_{H}DJ_{H} (62). Both of these lines express mouse or human surface IgM, respectively. Mice with silenced Igκ and Igλ loci have been obtained by gene targeting (7, 46). CD5 knock-out mice permit an assessment of a particular lymphocyte subpopulation, B-1a, featuring unusual specificities (63). Hox11 knock-out mice are spleenless (64) which addresses the question of whether spleen dependent events are important to secure the generation of cells that release H-chain-only antibodies. Animals were crossed to homozygosity for the analysis of IgH expression without L-chain and the following new combinations were obtained: μNR CD5^{-/-}, μNR Hox11^{-/-}, Hox11^{-/-} L^{-/-} and ΔVμ L^{-/-}. The μNR L^{-/-} mice had been obtained previously (see Example 1).

Surface Assembly of IgM without BiP-Retention Domain and L-Chain

As shown in Example 1, spontaneous IgG expression without L-chain is possible in mice when the first Cγ exon is removed during switch-recombination. The role of Cμ and whether surface expression needs to be achieved at the transitional B cell stage before switching is unclear. For a μ H-chain to be expressed on the B cell surface without L-chain beyond the preB cell stage may require the BiP-retention domain to be removed. Analysis of B220⁺ IgM⁺ bone marrow cells from normal, μNR and μNR L^{-/-} mice showed a reduction when C_{H}1 and C_{H}2 were missing, which was further reduced with the absence of the L-chain (FIG. 9). The increased numbers of B220⁺CD43⁺ cells in μNR L^{-/-} compared with μNR appear to indicate a bottleneck at the transitional stage when the preBCR is replaced by the BCR. Despite the reduction in the generation of IgM⁺ immature B cells, truncated surface IgM without L-chain is successfully expressed in μNR L^{-/-} mice. Furthermore Igβ can be detected on the surface of B220⁺ bone marrow cells from μNR L^{-/-} mice, while it is not detected in L^{-/-} mice suggesting an association of truncated μ with Igβ.

In both μNR and μNR L^{-/-} mice, the levels of CD5⁺ B cells in the bone marrow are increased compared to normal mice, which agrees with previous observations that a shorter μ chain permits expansion of this particular B cell subset (17). However, this is not seen in bone marrow cells from μNR Hox11^{-/-} compared to Hox11^{-/-} mice (FIG. 9). The μNR phenotype in this background does not appear to elicit an increase of this particular B cell population. Therefore, this finding underscores the pivotal role of the spleen in the generation of circulating B-1a cells, which re-enter the bone marrow as a distinct B cell population linking the innate and adaptive immune system (66) even when exclusively expressing H-chain-only antibodies.

The lack of the CD5 survival signal, which is important for negative BCR feedback signaling and stimulation of IL-10 production (67), might be expected to reduce the number of cells that express truncated IgM. Analysis of the bone marrow from μNR CD5^{-/-} mice (FIG. 9) found the opposite, with a doubling of B220⁺ and IgM⁺ cells, which was identified in a representative comparison of age-matched mice analyzed in parallel. This finding may be explained by the observation that a lack of CD5 expression can increase the capacity of B-1 cells to proliferate (68). In the spleen the levels of B220⁺ IgM⁺ cells were the same with or without CD5 (FIG. 10).

Developmental Progression from Primary to Secondary Lymphoid Organs

It was found that μNR chains were not only able to be expressed on the cell surface in the absence of L-chains, but were also efficient in supporting B cell development in the periphery. This is documented in the spleen by the occurrence of CD21/35⁺ cells, which are 11.9% in μNR L^{-/-} or about ⅓ of what is found in μNR mice and ¼ of the numbers in normal mice (FIG. 10a). Anti-Igβ staining indicated the presence of a BCR consisting of H-chain and co-receptor polypeptides but no L-chain.

Previous studies of peritoneal lymphocytes showed that the level of CD5⁺ B-1a cells is increased in μNR mice and that their lack in L^{-/-} mice is accompanied by the appearance of larger or differently shaped cells (17, Example 1 above). To observe the development of these cells and the potential importance of the CD5 cell surface marker in the selection process we analyzed μNR CD5^{-/-} animals. The majority of peritoneal cells were found in the conventional lymphocyte gate (www.flowjo.com; 19, 20) with the lack of CD5 not altering the B220⁺ B cell population (FIG. 10b). A similar result was seen in the spleen (FIG. 10a) with the levels of B220⁺ cells being maintained independent of CD5 expression.

Spleenless (Hox11^{-/-}) mice are known to lack most of the peritoneal B-1a cell population (66). Accordingly, we found a decrease in the percentages of CD5⁺ B cells in the peritoneum of those mice. Interestingly, in μNR Hox11^{-/-} the CD5 B cell levels were as high as in μNR or normal mice (FIG. 10b). This may suggest that peritoneal B-1 cells, unlike bone marrow B cells, can be maintained in spleen-independent fashion in certain circumstances. This is supported by the suggestion that peritoneal and splenic B-1 cells may be separate subpopulations (69); the selection of their fate being driven by signal strength of the BCR (70), in this case the truncated μ produced by μNR mice.

### H-Chain-Only Ig Secretion is Independent of the Spleen

Although the spleen plays a major role in disease protection, and splenectomized patients and mice respond poorly to some immunizations, this was not seen in Hox11 mice (71). Flow cytometry confirmed that developmental progression and cell levels in the bone marrow, from the preB to the immature stage, are quite similar between Hox11^{-/-} and normal mice although moderate variations, mostly small reductions, do occur (FIG. 9). To gain information about the cells that release H-chain-only antibodies in L^{-/-} mice (see Example 1), and whether the spleen provides the microenvironment to generate these antibodies, serum from Hox11^{-/-} L^{-/-} mice was analyzed. As shown in FIG. 11a H-chain-only IgG levels are only slightly reduced in Hox11^{-/-}L^{-/-} compared to L^{-/-} mice, with some mice having equal levels, suggesting that the spleen is not essential to produce H-chain-only antibodies. A possible reason could be that the production of H-chain-only IgG is initiated in the bone marrow, perhaps by early class-switching from IgM to IgG, followed by migration or homing to secondary lymphoid organs such as the peritoneal cavity. The controls in FIG. 11a show the expected high level of IgG in Hox11^{-/-} mice comparable to that found in normal mice, which is in agreement with previous studies of immunized animals (71).

A detailed comparison of antibody levels found in the various knock out and transgenic mouse lines is given in FIG. 11b. The analysis of serum IgM, IgG, Igκ and Igλ shows that transgenic ΔVμ, CD5^{-/-} and normal mice have comparable high antibody titers, whilst μNR, Hox11^{-/-}, μNR CD5^{-/-} and μNR Hox11^{-/-} have slightly reduced levels. CD5 deficiency in μNR mice increased the levels of B220⁺ IgM⁺ bone marrow cells but the amount of serum IgM and IgG remained the same. All the L-chain deficient mice in different backgrounds, μNR L^{-/-}, Hox11^{-/-} L^{-/-} and ΔVμ L^{-/-}, produce up to a few hundred μg/ml H-chain-only IgG, similar to L^{-/-} animals. In addition, up to 1 mg/ml of IgM is produced in μNR L^{-/-} mice and these high levels of truncated IgM lacking L-chain are very similar to the IgM levels of animals with fully functional L-chain.

Autonomous Expression of a HCD μ Transgene without L-Chain

The finding that μ H-chains lacking C_{H}1 and C_{H}2 were present on the cell surface without L-chain raised the question whether other truncated H-chains can be independently expressed or need the presence of a conventional L-chain during development. In ΔVμ transgenic mice the HCD-like human μ polypeptide without V_{H}DJ_{H} (FIG. 8) can be expressed on the cell surface apparently in the absence of L-chain, although L-chain rearrangement has occurred (62). However, this does not rule out the possibility that L-chain facilitates expression without association.

To ascertain whether ΔVμ polypeptides can sustain B cell development in the absence of L-chain breeding to homozygosity was established in the L^{-/-} background. Serum analysis showed that the levels of H-chain-only IgG in ΔVμ L^{-/-} mice are similar to those found in L^{-/-} mice (FIG. 11b). No ΔVμ IgM was detected in ΔVμ L^{-/-} mice which was as expected as levels were already very low in ΔVμ transgenic animals due to anergy or unresponsiveness to stimulation of this molecule. Also the lack of L-chain had little or no effect on preB cell development as assessed by c-kit and CD43 staining of bone marrow B220⁺ cells. This was also anticipated, as L-chains are not produced at the preBCR stage. Interestingly, a distinct second B220⁺ preB cell population is present in the bone marrow of ΔVμ L^{-/-} mice, which shifts upon staining with anti-human IgM (FIG. 12 left, indicated by arrows). Significantly, the numbers of splenic B220⁺ cells were equivalent in the absence or in the presence of L-chain in ΔVμ mice, while endogenous mouse IgM⁺ cells were not found in the absence of L-chain (FIG. 12 right). These results provide further support for the view that truncated H-chain, with V_{H} or C_{H}1 removal, do not stably associate with BiP, which permits cellular release and surface deposition.

### 2.3 Discussion

A comparison of novel mouse lines with altered lymphocyte populations and silenced Ig genes, revealed that developmental processes without L-chain are adequately maintained if a truncated μ H-chain reaches the cell surface. The finding complements the reports that full length μ H-chains, deposited on the cell surface without surrogate or conventional L-chain, fail to secure B cell maturation (58-61). In camelids, H-chain-only IgG is produced from particular Cγ genes with seemingly conventional C_{H}1 exons not included in the transcribed RNA (29, 49). In lower vertebrates H-chain dimers have been recognized that lack a classical C_{H}1-type domain, important to provide the cysteine residue for the disulphide linkage with L-chain (11, 12). Expression of truncated single-chain IgM or IgG in transgenic mice established that the lack of C_{H}1 is essential to secure B cell maturation (27, 28). The adaptations found in naturally produced mouse H-chain-only IgG in L^{-/-} animals are similar to single chain Ig in camelids and cartilaginous fish (see Example 1). Surface expression of this novel BCR without L-chain appears to include the co-receptor molecules, unambiguously shown for Igβ in FIG. 10. However, surface H-chain-only Ig could also be expressed independent of Igα/Igβ via conventional GPI (glycosyl-phosphatidyl-inositol) linkage (18). In the absence of L-chain the lack or modification of C_{H}1 is essential for unhindered transport through the ER, assembly in dimeric form and secretion, as cellular retention relies on non-covalent C_{H}1-association of a full length nascent-translated H-chain with the H-chain binding protein BiP (15, 30).

Chaperone-interaction is part of the quality control machinery in the ER with BiP providing one of several partners that associate with the Ig H-chain before interaction with L-chain and disulphide-linkage (72). In the absence of L-chain BiP remains bound to C_{H}1 (55) and upon disassociation other members of the H-chain-BiP-chaperone complex such as the most abundant GRP94 (54, 72) may complete the protein folding reaction. A high ratio of occupied BiP may activate UPR-control genes (21, 53) and it is conceivable that the inability of H-chain-only Ig to associate with BiP may prevent H-chain degradation. Alternatively, the synthesis level of truncated H-chain may exceed the degradation capacity. It may be possible to test this and preliminary evidence in older mice suggests that μNR L^{-/-} mice have a tendency to generate Russell bodies, an Ig oligomerisation aggregate (Mattioli et al., 2006). Nevertheless, an H-chain lacking one or two complete domains appears to retain the capacity for correct folding and disulphide linkage formation, which secures transport and release from the ER. This reiterates the remarkable stability of a basically incomplete H-chain polypeptide and suggests that chaperones recognize primarily structures within intact domains, thus permitting the transport of proteins with undamaged but variable numbers of subunits. A shorter or truncated H-chain may simply be processed equivalent to the L-chain, which can be expressed at a substantially higher level than full length H-chain in the same cell (74).

Since plasma cells can produce exclusively H-chain transcripts of reduced size in the spleen (see Example 1) it was unexpected to find that H-chain-only Ig levels were secured in spleen-less Hox11^{-/-} L^{-/-} mice. However, B cell maturation may be similar for conventional and H-chain-only antibodies; starting in the bone marrow, followed by migration and maturation in the periphery, and the colonization of secondary lymphoid organs. This would explain why the occurrence of H-chain IgM does not appear to establish the malignant phenotype seen in human HCD. The structural similarities between H-chain antibodies and HCD drew attention to the unresolved issue whether a functional L-chain locus is implicated in the release of HCD protein. Comparing the expression of ΔVμ and μNR polypeptides in the L^{-/-} background established that cellular exclusion is L-chain independent for both types of truncation, a lack of C_{H}1 or in the case of DVμ and presumably other HCD proteins, deletion of V_{H}DJ_{H}. In the DVμ mice transcripts lacking C_{H}1 were not found, although sometimes a few residues are missing at the leader-C_{H}1 splice junction identified by RT-PCR (62). Therefore it seems possible that a protein lacking VDJ can escape BiP-dependent retention instead of raising synthesis levels to exceed the degradation capacity.

The finding that the CD5 transmembrane glycoprotein is in association with IgM on the cell surface (75) may provide information regarding the differentiation potential of μNR cells expressing an incomplete IgM. CD5 is seen as a negative regulator of B-1a cell activation and CD5⁻ B-1 cells show reduced apoptosis upon BCR ligation, which led to the suggestion that induction of CD5 by autoantigen may be a mechanism to avoid undesired specificities (76, 77). As monovalent IgM from μNR mice is unable to recognize antigen, the protective function of CD5 may not be utilized compared with animals producing conventional IgM. CD5⁻ cells expressing such incomplete IgM appear to have an advantage in early development, possibly because of a more favorable proliferation signal, although disadvantages may arise after class-switching when conventional, fully functional isotypes are produced in these animals (17). Shortcomings may be revealed upon immunization and CD5 expression could be important for the selection of high affinity antibodies (78).

In summary, our analysis of mouse lines with compromised BCR show how robust and versatile antibody production is in the mammalian immune system. H-chain-only Ig expression in mice is comparable to relatively recent adaptations in camelids (13) and expression of single H-chains in cartilaginous fish, which may be a remnant of the primordial antibody structure that preceded the heterodimeric association of H- and L-chains (11). Cellular transport and release is accomplished because chaperone-recognition, which determines processing, retention and degradation, does not recognize H-chain IgM lacking distinct domains—C_{H}1 and C_{H}2 in μNR and VDJ in ΔVμ—as a misfolded protein. This secures surface deposition, which initiates feedback signals to progress B cell development leading to H-chain-only antibody secretion.

### EXAMPLE 3

In healthy individuals single Ig heavy chains cannot be released from the cell because intracellular transport of Ig is only achieved upon correct folding and assembly in the endoplasmic reticulum (ER). A single H-chain of any isotype is chaperoned by association with the H-chain binding protein, BiP or grp78 (83), which is then displaced by L-chain, allowing translocation to the cell surface or secretion. However, the release of single chain IgA is observed in human αHCD. In this case, removal of V_{H} and/or C_{H}1 exons precludes BiP association, facilitating translocation and secretion.

In this example, we show that L^{-/-} mice surprisingly generate a novel class of H-chain only Ig with covalently linked α-chains, not identified in any other healthy mammal. Also unexpectedly, diverse H-chain-only IgA can be released from B cells at levels similar to conventional IgA and is found in serum, milk and saliva. Surface IgA without L-chain is expressed in B220⁺ spleen cells, which exhibited a novel B cell receptor and suggested that associated conventional differentiation events occur. To facilitate the cellular transport and release of H-chain-only IgA, chaperoning via BiP-association seems to be prevented as only α-chains lacking C_{H}1 are released from the cell. This appears to be accomplished by exon deletion as a result of imprecise class-switch recombination, which removes all or part of Cα1 at the genomic level.

### 3.1 Materials and Methods

### Mouse Strains

The derivation of Igκ and Igλ deficient (L^{-/-}, with Cκ disrupted by neo insertion and a ˜120 kb region from Cλ2 to Cλ1 removed by targeted integration and Cre-IoxP deletion), C deletion (CΔ, lacking a ˜200 kb region from Cμ to 3′ of Cμ removed by targeted integration and Cre-IoxP deletion) and μMT mice has been described (7, 45, 6). L^{-/-} and μMT animals were crossbred to homozygosity. Mice ranging from 2 to 14 months old were analysed.

### ELISA of Body Fluids

Serum antibodies were analyzed as described (46) on Falcon plates coated with 15 μg/ml anti-mouse IgA (Sigma M8769). Biotinylated detection antibody, anti-mouse IgA (Sigma B-2766), was used at a 1:300 dilution, followed by incubation with 1:300 streptavidin biotinylated horseradish peroxidase (HRP) (Amersham RPN 1051V). Antibodies in milk, saliva, urine and faeces were analyzed in the same way. For this a known mass (weight/volume) of faeces or milk (taken from the stomachs of pups) sample was dissolved in an equivalent volume (50-100 μl) of phosphate-buffered saline (PBS). Saliva from swabs was also taken into PBS (50 μl).

### Western Analysis and Mass-Spectrometry

Serum Ig was captured on anti-mouse Ig(μ, γ and α H-chain specific; SouthernBiotech 1010-01)—or, for mass-spectrometry, anti-mouse IgA (Sigma)-coupled Sepharose, separated on Ready-Gels (Bio-Rad) under reducing or non-reducing conditions and transferred to nitrocellulose membranes as described (27). Filters were incubated with biotinylated (B10) anti-mouse antibodies specific for IgA (Sigma) or Igκ (Zymed) and λ (BD Pharmingen) L-chain. This was followed by incubation with streptavidin biotinylated HRP (Amersham) and visualization of bands using SuperSignal West Pico chemiluminescent substrate (Pierce, Ill.). Protein MW standards were supplied by Bio-Rad.

For analysis by mass-spectrometry Coomassie-stained bands were destained, reduced, carbamidomethylated and digested overnight with 10 ng/μl Sequencing Grade Modified Trypsin (Promega) in 25 mM NH₄HCO₃ at 30° C. The resulting peptide mixtures were separated by reversed-phase liquid chromatography as previously described (86). Proteins were identified by database searching of the mass spectral data using Mascot software (Matrix Science).

### Flow Cytometric Analysis and Cell Sorting

Multi-color analyses and sorting were carried out on a BD FACSAria. For analysis of IgA-positive cells, spleen cell suspensions were prepared and cells were stained with anti-mouse IgA-BIO (Sigma), CD45R (B220)-allophycocyanin (APC), CD90-FITC and F4/80-FITC (BD Pharmingen). This was followed by incubation with PE-Cy5.5-conjugated streptavidin (eBioscience). FlowJo software was used for the analysis.

For sorting of plasma cells for genomic DNA analysis, spleen cells were stained with FITC-conjugated anti-CD45R and BIO-conjugated anti-CD138 (syndecan-1) (BD Pharmingen) followed by incubation with PE-Cy5.5-conjugated streptavidin (eBioscience).

### RT-PCR Analysis

RNA was isolated from tissue or cells using Trizol (Gibco-BRL) and reverse-transcribed at 42° C. with Omniscript (Qiagen) or Bioscript (Bioline) reverse transcriptase. PCR reactions with J_{H} and Cα3 primers were set up using KOD Hot Start DNA polymerase (Novagen), at a final MgSO₄ concentration of 0.8 mM. Primers are listed in Table 5 below. The cycling conditions were 94° C. for 2 min, 40 cycles of 94° C. for 15 sec, 59° C. for 30 sec, 72° C. for 15 sec, followed by 72° C. for 10 min; the β-actin control for semi-quantitative PCR required 26 cycles at an annealing temperature of 61° C. RT-PCR products were either cleaned up (DNace Quick Clean, Bioline) and sequenced directly or cloned by adding a 3′ A overhang and using a TA Cloning Kit (Invitrogen).

PCR reactions with Cα3 and degenerate V_{H} primers (Table 5) were set up as above, but annealing temperatures of 58° C. (J558, VH7183 and VGAM) or 52° C. (Vgen) were used, with a ramp speed of 1° C./sec. PCR products were purified from an agarose gel using the Wizard SV Gel PCR Clean-Up System (Promega) before cloning and sequencing. V_{H}, D and J_{H} genes were identified using the IMGT database (http://imgt.cines.fr). Agarose gels were run with size markers in by (100 by DNA ladder, Invitrogen).

### Genomic DNA Analysis

Genomic DNA was prepared and analyzed by long range PCR as described previously (86). A first round PCR of 20 cycles from J_{H}4L to Cα2L1 was followed by a nested second round PCR of 25 cycles from 3′ Eμ to Cα2L2 (primers listed in Table 5). Any bands obtained were cloned as described above or picked from the gel and re-amplified and sequenced.

### 3.2 Results

### Expression of High Levels of IgA in L^{-/-} Mice

ELISA discovered IgA titers in serum from L^{-/-} mice, which in some cases were very similar to the level of conventional IgA found in normal mice (FIG. 13A); in comparison with the low levels of IgG expression seen in L^{-/-} mice (86), it appears that H-chain IgA can be produced much more readily. This antibody class is not known to be secreted without L-chain in healthy animals; however the wellbeing of the L^{-/-} mice appears to be unaffected.

### Size and Configuration of H-Chain-Only IgA

To determine the molecular weight of the α H-chains, Western blot analysis was performed on Ig separated under reducing and non-reducing conditions (FIG. 13B,C). This shows α H-chains of ˜46 kDa, which is ˜15 kDa or one domain shorter than conventional α chains (FIG. 13B). H-chain IgA appears to share a common feature with H-chain IgG, in that the size of the H-chain is approximately one domain shorter than normal in both cases. The analysis of purified IgA polypeptides from gel slices by mass-spectrometry confirmed the lack of a single domain, as it revealed an extensive number of Cα sequences from C_{H}2 and C_{H}3, but not C_{H}1 (Table 6). Separation under non-reducing conditions (FIG. 13C) identified covalent linkage of 2 α H-chains with a molecular weight of ˜92 kDa and a low level of multimers, 4 covalently linked α H-chains, similar to multi-chain normal IgA (81). We were not able to identify covalent J-chain association, either in ELISA or Western blots, due to a lack of specific reagents. Its presence would add ˜15 kDa to the molecular weight of the tetramer; however this small difference cannot be resolved in the high molecular weight region of the gel.

### Lymphoid Tissues Express Truncated α H-Chain

The production of α H-chain transcripts in different tissues was compared by semi-quantitative RT-PCR (FIG. 14A). In normal mice transcripts from J_{H} to Cα3 are ˜850 bp, whereas in L^{-/-} mice the predominant band was ˜550 bp. The use of different J_{H} oligos, for J_{H}1, 2, 3 and 4, revealed the smaller product in all amplifications using RNA from bone marrow and spleen. Sometimes, the ˜550 by band was also obtained from lymph node and ileum preparations, for example in J_{H}2 to Cα3 reactions. Occasionally, an ˜850 by product was seen, presumably corresponding to a normal size transcript; however these signals were always weaker, indicating that the shorter product represents the major product in these tissues. Sequencing revealed that the shorter product encompasses a region from J_{H} to Cα3 without C_{H}1.

### Diverse V_{H}, D, J_{H} Usage in H-Chain IgA

The analysis was extended to gain information about the V_{H} gene repertoire of H-chain-only antibody transcripts. Amplification with different V_{H} family oligos (J558, VGAM and V7183) identified strong bands of ˜880 bp, representing α H-chains encompassing V_{H}-D-J_{H}-C_{H}2-C_{H}3 but not C_{H}1 (FIG. 14B); similar products were also seen upon amplification with the more degenerate V_{H}-gene primer, Vgen (data not shown). These products were cloned and sequenced, allowing identification of several different V_{H}, D and J_{H} segments from each mouse and also showing that J_{H} is correctly spliced to C_{H}2 and in one case to C_{H}3 (FIG. 20). The full-length product of ˜1150 by was the main band found in normal mice. However, a truncated product, represented by a smaller band of weaker intensity, was also present and may indicate a spontaneous mechanism to produce truncated α H-chains.

Analysis of the V_{H} domain showed diverse D and J_{H} rearrangement with the addition of non-encoded residues at the junctions and extensive alterations by hypermutation (FIG. 20). Interestingly, several of the V_{H} sequences carry a high level of replacement residues, suggesting antigen-dependent selection, and the high ratio of transition to transversion mutations is a well-established feature of the somatic hypermutation mechanism (90, 91). The diversity of V_{H} genes used in the H-chain-only antibodies was confirmed by the results of mass spectrometry: V_{H} sequences from four different families, VH7183, J558, VGAM3-8 and 3609, were identified by their framework and CDR regions (Table 6).

To further investigate whether antibody specificities could potentially be selected we stained the surface of spleen cells with anti-IgA. As can be seen in FIG. 15, after T-cell and macrophage exclusion a small but distinct population of IgA⁺ B220⁺ cells (1.4%) can be identified. This population could not be readily distinguished in all animals analyzed, however when mice displaying higher serum IgA titers were selected, the data was found to be reproducible. As no conventional L-chain is produced in these mice (86, 7), this is the first example of spontaneous expression of a new type of BCR without L-chain on mature B cells, which forms a further aspect of the present invention.

### H-Chain IgA Secretion and Excretion

The varied H-chain-only IgA titer in L^{-/-} mice prompted a detailed analysis of age-related and environmental constrains which could drive expression. The questions we addressed were: Do older animals produce higher Ig levels, and does an open or closed, pathogen-free, animal facility bias the expression? A comparison of IgA levels at 100-fold serum dilution is presented in FIG. 16, which shows the predicted range for conventional IgA from normal mice. The oldest two L^{-/-} mice housed in the closed facility, 9 and 8 months of age, gave the highest titers very similar to normal mice. Some 5 month-old mice had a medium titer and some younger mice had a low titer, but there were also many exceptions to this pattern. However, there does appear to be a propensity favoring higher expression of H-chain IgA in older mice. This trend also occurs in animals kept in open or easily accessible facilities, as only older mice have the highest titer. Overall, the antibody serum levels in open and closed facilities appear to be similar and perhaps other events, e.g. small injuries or airborne contamination, which may accumulate with time, provide the essential immune stimulation to obtain high antibody titers.

In μMT C57BI/6 animals, IgA is expressed at various levels and seemingly independent of IgM and IgD (92). To test whether H-chain-only IgA can be expressed independently we crossed L^{-/-} mice with μMT animals to homozygosity. As shown in FIG. 16 (left) no IgA could be identified in the serum of μMT L^{-/-} animals. The lack of IgA and the previous finding that no H-chain-only IgG is produced when Cμ is disrupted (86) suggests that preBCR and/or surface IgM expression is required for H-chain-only antibody expression in L^{-/-} mice.

The protective function of IgA plays a central role in mucosal immunity, and is often the first point of contact between the antigen and the adaptive immune system (81). IgA is also abundant in secretions, including milk and colostrum, and provides a vital source of neonatal immunity (93). To gain information as to whether H-chain-only IgA can fulfill these roles, we analyzed milk, saliva, urine and faeces from L^{-/-} mice by ELISA (FIG. 17). In normal mouse controls excreted IgA was easily detectable in all samples, whereas intermittent release was found in L^{-/-} mice. In general, release of H-chain-only IgA via the mucosal route was rare and only seen in some L^{-/-} mice with higher serum Ig levels; for example, the L^{-/-} mouse with the highest score in saliva (FIG. 17) has an ELISA reading of 2 in FIG. 16 (central panel).

### Removal of C_{H}1 by Imprecise Class-Switch Recombination

A lack of the C_{H}1 exon, identified in protein and transcriptional analyses of H-chain-only IgA, could be the result of either alternative splicing of RNA transcripts or genomic alteration during B cell maturation. We have looked at whether genomic deletions produce Cα regions devoid of C_{H}1 by employing a long range PCR approach using sorted syndecan-positive plasma cells as described recently (86). Class-switch recombination from Cμ [via Cγ] to Cα retains only the last C gene and juxtaposes the rearranged V_{H}DJ_{H} less then 10 kb upstream of Cα (94). FIG. 18A illustrates the gene layout after switching, indicating the position of the oligos for the initial PCR amplification from J_{H}4L to Cα2L1 followed by a further nested PCR amplification with oligos from 3′Eμ to Cα2L2. The nested amplification bands and the sequence information for the indicated bands obtained after cloning are illustrated in FIGS. 18B and C. In L^{-/-} mice smaller distinct fragments occur, which appear to indicate instability or deletion in this region. Indeed the sequence of the PCR bands from L^{-/-} mice revealed a large number of deletions, which had various parts of the switch region and Cα1 removed. Sequencing of larger products showed an apparently intact Cα1 in some cases. Cloning of the normal mouse PCR products in the 2 kb range did not in general show a lack of Cα1 and the obtained sequences included Cα1, 5′ Cα1 and a switch region encompassing switch-μ, -γ and -α sequences (see FIGS. 21 and 22). However, a deletion encompassing part of Cα1 was observed in one case, suggesting that mechanisms leading to exon deletion do occur in normal mice, but are only selected for in L^{-/-} animals. As the deletions found in L^{-/-} mice remove all of Cα1 or the 5′ end of the exon, this would explain the presence of IgA transcripts lacking C_{H}1. Since a large part of the upstream switch sequence is also lost, it is conceivable that DNA lesions during switch recombination (25) result in these Cα alterations, which facilitate H-chain-only IgA expression.

TABLE 5
Primer sequences.

| Primer | Sequence (5′-3′) |
|---|---|
| J_{H}1 | CGGTCACCGTYTCCTCAG |
| J_{H}2 | GCACCASTCTCACAGTCTCCT |
| J_{H}3 | GGGACTCTGGTCACTGTCTCT |
| J_{H}4 | AACCTCAGTCACCGTCTCCTC |
| J_{H}4L | GGAACCTCAGTCACCGTCTCCTCAG |
| 3′ Eμ | GCACTGACCACCCGGAG |
| J558 | SAGGTYCARCTSCARCAGYCTGG |
| VH7183 | GAMGTGMAGCTSKTGGAGWCTGG |
| VGAM | CAGATCCAGTTSGTRCAGTCTGG |
| Vgen | SADGTBCAGCTKMAGSAGTCWGG |
| Cα3 | GCTCCTTTAGGGGCTCAAAC |
| Cα2L1 | CAGGCAGGACGCTGGACACA |
| Cα2L2 | ACTGTAGCAGCCGCAGGAAT |
| β-actin F | GATATCGCTGCGCTGGTC |
| B-actin R | CTACGTACATGGCTGGGGTG |

### Mass-Spectrometry Experimental Details

Serum IgA from two L^{-/-} mice was purified by binding to anti-mouse IgA-conjugated sepharose, and size separated by SDS-PAGE, as can be seen in FIG. 23. Mass-spectrometry, after trypsin digest of the bands indicated (1, 2, 3 and 4), identified a total of 11 different peptides listed in Table 6 (9 in mouse 1 and 10 in mouse 2) within the C_{H}2 and C_{H}3 exons of IgA; no fragments within C_{H}1 were found. For V_{H} sequences, framework and CDR regions were identified for genes from the following families listed in Table 6. Note that peptides in brackets were matched to database entries, but differ only in K/Q substitutions, which are not distinguished under the mass spectrometry conditions used.

TABLE 6

| Exon | Peptide sequence |
|---|---|
| C_{H}2 - | PALEDLLLGSDASITCTLNGLR |
| | NPEGAVFTWEPSTGKDAVQK |
| | KAVQNSCGCYSVSSVLPGCAER |
| | WNSGASFK |
| | CTVTHPESGTLTGTIAK |
| C_{H}3 - | VSAETWK |
| | QGDQYSCMVGHEALPMNFTQK |
| | LSGKPTNVSVSVIMSEGDGICY |
| | AFNPKEVLVR |
| | EPGEGATTYLVTSVLR (mouse 1 only) |
| CH2 + 3 - | VTVNTFPPQVHLLPPPSEELALNELLSLTCLVR (mouse |
| | 2 only) |
| VH7183 - | LVESGGGLVKPGGSLK |
| | EVQLVESGGGLVK |
| | EVQLVESGGGLVKPGGSLK |
| | (EVKLVESGGGLVQPGGSLK) |
| | (EVKLVESGGGLVKPGGSLK) |
| | NTLYLQMSSLK |
| | NTLYLQMNSLK |
| | NNLYLQMSSLK |
| | NILYLQMSSLR |
| | SEDTAMYYCAR |
| | LSCAASGFAFSSYDMSWVR |
| | RLEWVAYISSGGGSTYYPDTVK |
| J558 - | ATLTVDK |
| | EVQLQQSGPELVKPGASVK |
| | QLKLQESGPELVK |
| | QVQLQQSGPELVKPGASVK |
| | QVQLQQXGAELVKPGASVK |
| | SLEWIGR |
| | SLEWIGDINPNNGGTSYNQK |
| | ASGYTFTDYYMK |
| VGAM3-8 - | QIQLVQSGPELKK |
| | QIQLVQSGPELK |
| | QIQLVQSGPELKKPGETVK |
| | SEDTATYFCAR |
| 36-60 - | NQFFLK |
| 3609 - | YNPSLK |

For the identification of J chain, serum IgA from 5 L^{-/-} mice was separately captured by binding to anti-mouse IgA-conjugated sepharose and directly analysed by mass-spectrometry. This resulted in the identification of α-chain and J chain peptides denoted by underlining the region in the J chain sequence from Yagi et al. (J. Exp. Med. 155:647, 1982):

| | |
|---|---|
| 1 | MKTHLLLWGV LAIFVKAVLV TGDDEATILA DNKCMCTRVT |
| | SRIIPSTEDP |
| 51 | NEDIVERNIR IVVPLNNREN ISDPTSPLRR NFVYHLSDVC |
| | KKCDPVEVEL |
| 101 | EDQVVTATQS NICNEDDGVP ETCYMYDRNK CYTTMVPLRY |
| | HGETKMVQAA |
| 151 | LTPDSCYPD |

### 3.3 Discussion

Further study of L-chain deficient mice has revealed a new type of antibody, H-chain-only IgA, which is released from the cell and surface expressed. There are no examples of the occurrence of this isotype in Tylopoda or camelids, which produce H-chain-only IgG, or in elasmobranchs (sharks, skates and rays), where H-chain-only antibodies can comprise a variable number of Cμ domains (50, 88). A common feature of murine H-chain-only IgA, as well as other naturally occurring H-chain antibodies, is the lack of a typical C_{H}1 domain. As a result the shortened nascent-translated H-chain cannot form an association complex with the H-chain binding protein BiP as interacting C_{H}1 residues are lacking (83, 30). The immediate advantage is that α H-chain without C_{H}1 secures unhindered transport through the ER leading to surface deposition and H-chain-only antibody secretion (86, 95). Unexpectedly, H-chain IgA is remarkably stable, degradation seems to be prevented, and protein levels are sizeable, in some cases almost reaching conventional IgA levels in the mouse. Flow cytometry and RT-PCR identified spleen lymphocytes as a major source of α H-chain transcripts lacking C_{H}1, which is in agreement with the recent findings of short γ transcripts in syndecan⁺ plasma cells. In addition, long-range PCR using DNA from sorted spleen cells identified prominent deletions in which the switch sequence and part or all of Cα1 was removed, which is similar to findings for murine H-chain-only IgG (86). However, the expression level of H-chain-only IgA and the abundance of a transcripts lacking C_{H}1 as a dominant band in RT-PCR amplifications suggest that expression of this particular isotype is much more readily achieved. This may be due to a large number of staggered consensus repeats of the α switch region and proximal control elements such as the 3′ enhancer downstream of Cα1, a combination which may intrinsically favour aberrant switching (94, 96).

Our analysis of genomic DNA from normal mice also revealed the presence of products lacking Cα1, indicating that the mechanisms leading to heavy-chain-only antibody production exist in the normal situation (when L-chains are expressed); this is supported by the presence of a weak lower band in some RT-PCR analyses (FIG. 14B). However, in L^{-/-} mice the selection pressure favoring B cells producing such antibodies enables them to be much more readily detected.

It has been reported that IgA may be expressed independently of IgM or IgD in early ontogeny, which could be an evolutionary primitive system that does not rely on class-switching from μ to a downstream isotype(92). As our results are consistent with the notion that H-chain-only IgA is produced and secreted by the same B cell subset as conventional IgA, we asked whether H-chain-only IgA can be expressed in the μMT background, which provides a B cell block due to a lack of surface IgM production (6). This does not seem to be the case and no H-chain IgA or any other isotype has been detected in μMT L^{-/-} animals (see FIG. 16 and ref 86), which implies that IgM expression during ontogeny is probably essential to progress developmental events that allow C-gene modification followed by H-chain expression.

In camelids, the V_{H}H genes used in H-chain-only antibodies often contain specific alterations such as hallmark amino acids or an extended CDR3 region, both to compensate for the lack of L-chain, and to prevent L-chain association in a system in which both H-chain-only and conventional antibodies are produced (97, 40, 41, 42). Comparison of mouse α H-chain V_{H}s with camelid V_{H}Hs did not, however, reveal the presence of similar alterations. This matches the finding with mouse γ H-chain V_{H}s and reflects the fact that murine V_{H}s have not been selected in evolution to be optimal for H-chain-only antibody production. However, it also suggests that there are fewer restrictions in the sequence of the mouse variable region; this is probably due to the complete absence of the L-chain in these mice. Therefore, murine H-chain IgA and similar H-chain only IgA binding molecules of the invention could be structurally different from the configuration of camelid H-chain IgG, and it is conceivable that two antigen-binding moieties, each contributed by a H-chain, may associate to form a single antigen-binding site. Indeed, certain V_{H} gene sequences may be advantageous to permit different formats and the capacity of V_{H} domains to dimerise spontaneously is not unusual (98).

Possible configurations of H-chain-only IgA according to the invention are illustrated in FIG. 19. The dimeric and tetrameric assembly is disulphide-linked and may include the J chain. An associated dimeric configuration of V_{H}-domains has been described (98) and an example of such a linkage binds to DNA (99). Interestingly, conventional monoclonal IgA anti-DNA or auto-antibodies can be readily isolated after fusion of Peyer's patch cells and key amino acids in their CDR regions have been related to this specificity (100). We have observed that L^{-/-} mice lack visible Peyer's patches, the factory for IgA produced by the mucosal immune system to combat air- or food-born pathogens such as viruses or bacteria, although truncated IgA transcripts have been found in the ileum.

The structural differences between H-chain-only and conventional IgA raised the question of whether the truncated polypeptide, without L-chain, would be recognized by the polymeric Ig receptor (or secretory component), allowing its release from mucosal surfaces. This receptor is produced in epithelial cells separate from plasma cells secreting serum IgA. Its specificity for polymeric Ig (101) implied that the level of IgA secretion into external fluids would be lower in L^{-/-} mice as the extent of IgA oligomerization is reduced. This is indeed the case (see FIG. 17) and our results indicate that whilst sometimes H-chain-only IgA is found in secretions, this is not usually so, even when the serum level of IgA is high.

Previously, production of H-chain-only IgA had only been observed in the context of disease. In L^{-/-} mice spontaneous expression of H-chain IgA clearly differs from human αHCD, because the animals appear healthy with normal life expectancy in a pathogen-free environment. No invasion of plasma cells has been observed, which would cause lymphomas characteristic of HCD (84). Regular features associated with HCD involve deletions and insertions in the rearranged V_{H} genes (43), neither of which have been found in V_{H}DJ_{H} sequences from L^{-/-} mice. However, changes to permit cellular transport are common in both systems. In L^{-/-} mice the absence of L-chain leads to the selection and subsequent expansion of cells producing mutant α H-chains, which have lost the use of C_{H}1.

Although the present invention has been described with reference to preferred or exemplary embodiments, those skilled in the art will recognize that various modifications and variations to the same can be accomplished without departing from the spirit and scope of the present invention and that such modifications are clearly contemplated herein. No limitation with respect to the specific embodiments disclosed herein and set forth in the appended claims is intended nor should any be inferred.

### REFERENCES

1. Hardy, R. R., C. E. Carmack, S. A. Shinton, J. D. Kemp, and K. Hayakawa. 1991. Resolution and characterization of pro-B and pre-pro-B cell stages in normal mouse bone marrow. J. Exp. Med. 173:1213-1225.
2. Osmond, D. G., A. Rolink, and F. Melchers. 1998. Murine B lymphopoiesis: towards a unified model. Immunol. Today. 19:65-68.
3. Kearney, J. F. 2004. Development and function of B-cell subsets. In Molecular Biology of B-cells. T. Honjo, F. W. Alt, and M. S. Neuberger, editors. Elsevier Academic Press, Amsterdam, 155-160.
4. Melchers, F. and P. Kincade. 2004. Early B cell development to a mature, antigen-sensitive cell. In Molecular Biology of B-cells. T. Honjo, F. W. Alt, and M. S. Neuberger, editors. Elsevier Academic Press, Amsterdam, 101-126.
5. Pike, K. A., and M. J. H. Radcliffe. 2002. Cell surface immunoglobulin receptors in B cell development. Semin. Immunol. 14:351-358.
6. Kitamura, D., J. Roes, R. Kuhn, and K. Rajewsky. 1991. A B cell-deficient mouse by targeted disruption of the membrane exon of the immunoglobulin p chain gene. Nature. 350:423-426.
7. Zou, X., T. A. Piper, J. A. Smith, N. D. Allen, J. Xian, and M. Brüggemann. 2003. Block in development at the pre B-II to immature B-cell stage in mice without Igκ and Igλ L-chain. J. Immunol. 170:1354-1361.
8. Pelanda, R., U. Braun, E. Hobeika, M. C. Nussenzweig, and M. Reth. 2002. B cell progenitors are arrested in maturation but have intact VDJ recombination in the absence of Ig-α and Ig-β. J. Immunol. 169:865-872.
9. Hamers-Casterman, C, T. Atarhouch, S. Muyldermans, G. Robinson, C. Hamers, E. B. Songa, N. Bendahman, and R. Hamers. 1993. Naturally occurring antibodies devoid of L-chains. Nature. 363:446-448.
10. Padlan, E. A. 1994. Anatomy of the antibody molecule. Mol. Immunol. 31:169-217.
11. Greenberg, A. S., A. L. Hughes, J. Guo, D. Avila, E. C. McKinney, and M. F. Flajnik. 1996. A novel “chimeric” antibody class in cartilaginous fish: IgM may not be the primordial immunoglobulin. Eur. J. Immunol. 26:1123-1129.
12. Rast, J. P., C. T. Arnemiya, R. T. Litman, S. J. Strong, and G. W. Litman. 1998. Distinct patters of IgH structure and organization in a divergent lineage of chrondrichthyan fishes. Immonogenetics. 47:234-245.
13. Nguyen, V. K., C. Su, S. Muyldermans, and W. van der Loo. 2002. H-chain antibodies in Camelidae; a case of evolutionary innovation. Immunogenetics. 54:39-47.
14. Alexander, A., M. Steinmetz, D. Barritault, B. Frangione, E. C. Franklin, L. Hood, and J. N. Buxbaum. 1982. γ Heavy chain disease in man: cDNA sequence supports partial gene deletion model. Proc. Natl. Acad. Sci. USA. 79:3260-3264.
15. Haas, I. G., and M. Wabl. 1983. Immunoglobulin heavy chain binding protein. Nature. 306:387-389.
16. Munro, S., and H. R. Pelham. 1987. A C-terminal signal prevents secretion of luminal ER proteins. Cell. 48:899-907.
17. Zou, X. C. Ayling, J. Xian, T. A. Piper, P. J. Barker, and M. Brüggemann. 2001. Truncation of the μ heavy chain alters BCR signalling and allows recruitment of CD5⁺ B cells. Int. Immunol. 13:1489-1499.
18. Nguyen, V. K., X. Zou, M. Lauwereyes, L. Brys, M. Brüggemann, and S. Muyldermans. 2003. H-chain only antibodies derived from dromedary are secreted and displayed by mouse B-cells. Immunology. 109:93-101.
19. Roederer, M., W. Moore, A. Treister, R. R. Hardy, and L. A. Herzenberg. 2001. Probability binning comparison: a metric for quantitating multivariate distribution differences. Cytometry. 45:47-55.
20. Shapiro, H. M. 2003. Practical flow cytometry. 4^{th} edition. Wiley N Y.
21. Gass, J. N., K. E. Gunn, R. Sriburi, and J. W. Brewer. 2004. Stressed-out B cells? Plasma-cell differentiation and the unfolded protein response. TRENDS Immunol. 25:17-24.
22. Sanderson, R. D., P. Lalor, and M. Bernfield. 1989. B lymphocytes express and loose syndecan at specific stages of differentiation. Cell Regulation. 1:27-35.
23. Bolland, D. J., A. L. Wood, C. M. Johnston, S. F. Bunting, G. Morgan, L. Chakalova, P. J. Fraser, and A. E. Corcoran. 2004. Antisense intergenic transcription in V(D)J recombination. Nat. Immunol. 5:630-637.
24. Corcoran, E. 2005. Immunoglobulin locus silencing and allelic exclusion. Sem. Immunol. 17:141-154.
25. Xu, Z., Z. Fulop, Y. Zhong, A. J. Evinger, H. Zan, and P. Casali. 2005. DNA lesions and repair in immunoglobulin class switch recombination and somatic mutation. Ann. NY Acad. Sci. 1050:146-162.
26. Chen, J. M. Trounstine, F. W. Alt, F. Young, C. Kurahara, J. F. Loring, and D. Huszar. 1993. Immunoglobulin gene rearrangement in B cell deficient mice generated by targeted deletion of the J_{H} locus. Int. Immunol. 5:647-656.
27. Zou, X., J. A. Smith, V. K. Nguyen, L. Ren, K. Luyten, S. Muyldermans, and M. Brüggemann. 2005. Expression of a dromedary heavy chain-only antibody and B cell development in the mouse. J. Immunol. 175:3769-79.
28. Janssens, R. S. Dekker, R. W. Hendriks, G. Panayotou, A. van Remoortere, J. K. San, F. Grosveld, and D. Drabek. 2006. Generation of H-chain-only antibodies in mice. Proc. Natl. Acad. Sci. USA. 103:15130-15135.
29. Brüggemann, M., J. A. Smith, M. J. Osborn, D. Corcos, X. Zou, V. K. Nguyen, and S. Muyldermans. 2006. H-chain-only antibody expression and B-cell development in the mouse. Crit. Rev. Immunol. 26:377-390.
30. Hendershot, L. M. 1990. Immunoglobulin heavy chain and binding protein complexes are dissociated in vivo by L-chain addition. J. Cell Biol. 111:829-837.
31. Kan, J. L., and M. R. Green. 1999. Pre-mRNA splicing of IgM exons M1 and M2 is directed by a juxtaposed splicing enhancer and inhibitor. Genes Dev. 13:462-471.
32. Monk, R. J., S. L. Morrison, and C. Milcarek. 1981. H-chain mutants derived from γ2b mouse myeloma: characterization of H-chain messenger ribonucleic acid, proteins, and secretion in deletion mutants and messenger ribonucleic acid in γ2a mutant progeny. Biochemistry. 20:2330-2339.
33. Kinoshita, K., J. Tashiro, S. Tomita, C. G. Lee, and T. Honjo. 1998. Target specificity of immunoglobulin class switch recombination is not determined by nucleotide sequence of S region. Immunity. 9:849-858.
34. De Genst, E., D. Saerens, S. Muyldermans, and K. Conrath. 2006. Antibody repertoire development in camelids. Dev. Comp. Immunol. 30:187-198.
35. Nguyen, V. K. 2002. Generation of heavy chain antibodies in Camelids. PhD thesis. Free University of Brussels, Belgium.
36. Tarlinton, D. M., M. McLean, G. B. Nossal. 1995. B1 and B2 cells differ in their potential to switch immunoglobulin isotype. Eur. J. Immunol. 25:3388-93.
37. Herzenberg, L. A. 2000. B-1 cells: the lineage question revisited. Imm. Rev. 175:9-22.
38. Cartegni, L., S. L. Chew, and A. R. Krainer. 2002. Listening to silence and understanding nonsense: exonic mutations that affect splicing. Nature Rev. 3:285-298.
39. Odegard, V. H., and D. G. Schatz. 2006. Targeting of somatic hypermutation. Nature Rev. Immunol. 6:573-583.
40. Muyldermans, S., C. Cambillau, and L. Wyns. 2001. Recognition of antigens by single-domain antibody fragments: the superfluous luxury of paired domains. Trends. Biochem. Sci. 26:230-235.
41. Wu, T. T., and E. A. Kabat. 1992. Possible use of similar framework region amino acid sequences between human and mouse immunoglobulins for humanizing mouse antibodies. Mol. Immunol. 29:1141-1146.
42. Lauwereys, M., M. Arbadi Ghahroudi, A. Desmyter, J. Kinne, W. Holzer, E. de Genst, L. Wyns, and S Muyldermans. 1998. Potent enzyme inhibitors derived from dromedary H-chain antibodies. EMBO J. 17:3512-20.
43. Cogné, M., C. Silvain, A. A. Khamlichi, and J. L. Preud'homme. 1992. Structurally abnormal immunoglobulins in human immunoproliferative disorders. Blood. 79:2181-95.
44. Litman, G. W., J. P. Rast, M. J. Shamblott, R. N. Haire, M. Hulst, W. Roess, R. T. Litman, K. R. Hinds-Frey, A. Zilch, and C. T. Amemiya. 1993. Phylogenetic diversification of immunoglobulin genes and antibody repertoire. Mol. Biol. Evol. 10:60-72.
45. Ren, L., X. Zou, J. A. Smith, and M. Brüggemann. 2004. Silencing of the immunoglobulin heavy chain locus by removal of all 8 constant region genes on a 200 kb region. Genomics. 84:686-695.
46. Zou, X., J. Xian, A. V. Popov, I. R. Rosewell, M. Müller, and M. Brüggemann. 1995. Subtle differences in antibody responses and hypermutation of λ L-chains in mice with a disrupted κ constant region. Eur. J. Immunol. 25:2154-2162.
47. Deverson, E., C. Berek, M. J. Taussig, and A. Feinstein. 1987. Monoclonal BALB/c anti-progesterone antibodies use family IX variable region heavy chain genes. Eur. J. Immunol. 17:9-13.
48. Yu, Y. P., F. Lin, R. Dhir, D. Krill, M. J. Becich, and J. H. Luo. 2001. Linear amplification of gene-specific cDNA ends to isolate full-length of a cDNA. Anal. Biochem. 292:297-301.
49. Nguyen, V. K., Hamers, R., Wyns, L., Muyldermans, S. 1999. Loss of splice consensus signal is responsible for the removal of the entire C(H)1 domain of the functional camel IgG2a H-chain antibodies. Mol. Immunol. 36, 515-524.
50. Litman, G. W., Anderson, M. K., Rast, J. P. 1999. Evolution of antigen binding receptors. Annu. Rev. Immunol. 17, 109-147.
51. Wang, Y. H., Stephan, R. P., Scheffold, A., Kunkel, D., Karasuyama, H., Radbruch, A., Cooper, M. D. 2002. Differential surrogate L-chain expression governs B-cell differentiation. Blood 99, 2459-2467.
52. Stavnezer, J., Kinoshita, K., Muramatsu, M., Honjo, T. 2004. Molecular mechanism of class switch recombination. In: Honjo, T., Alt, F. W., Neuberger, M. S. (Eds.), Molecular Biology of B-cells. Elsevier Academic Press, Amsterdam, pp. 307-326.
53. Gething, M. J. 1999. Role and regulation of the ER chaperone BiP. Semin. Cell Dev. Biol. 10, 465-472.
54. Kleizen, B., Braakman, I. 2004. Protein folding and quality control in the endoplasmic reticulum. Curr. Opin. Cell Biol. 16, 343-349.
55. Hendershot, L., Bole, D., Köhler, G., Kearney, J. F. 1987. Assembly and secretion of heavy chains that do not associate posttranslationally with immunoglobulin heavy chain-binding protein. J. Cell Biol. 104, 761-767.
56. Buxbaum, J. N., Alexander, A. 2001. H-chain-Disease. Williams Hematology 6th edition, eds. Beutler, E. et al., pp 1327-1336; New York: McGraw-Hill.
57. Corcos, D., Grandien, A., Vazquez, A., Dunda, O., Lorès, P., Bucchini, D. 2001. Expression of a V region-less B cell receptor confers a tolerance-like phenotype on transgenic B cells. J. Immunol. 166, 3083-3089.
58. Su, Y., Flemming, A., Wossning, T., Hobeika, E., Reth, M., Jumaa, H. 2003. Identification of a pre-BCR lacking surrogate L-chain. J. Exp. Med. 198, 1699-1706.
59. Schuh, W., Meister, S., Roth, E., Jäck, H.-M. 2003. Cutting edge: Signaling and cell surface expression of a μH chain in the absence of λ5: A paradigm revisited. J. Immunol. 171, 3343-3347.
60. Galler, G. R., Mundt, C., Parker, M., Pelanda, R., Mårtensson, I.-L., Winkler, T. H. 2004. Surface heavy chain signals down-regulation of the V(D)J-recombinase machinery in the absence of surrogate L-chain components J. Exp. Med. 199, 1523-1532.
61. Geraldes, P., Rebrovich, M., Herrmann, K., Wong, J., Jäck, H. M., Wabl, M., Cascalho, M. 2007. Ig heavy chain promotes mature B cell survival in the absence of L-chain. J. Immunol. 179, 1659-1668.
62. Corcos, D., Iglesias, A., Dunda, O., Bucchini, D, Jami, J. 1991. Allelic exclusion in transgenic mice expressing a heavy chain disease-like human μ protein. Eur. J. Immunol. 21, 2711-2716.
63. Tarakhovsky, A., Müller, W., Rajewsky, K. 1994. Lymphocyte populations and immune response in CD5-deficient mice. Eur. J. Immunol. 24, 1678-1684.
64. Dear, T. N., Colledge, W. H., Carlton, M. B., Lavenir, I., Larson, T., Smith, A. J., Warren, A. J., Evans, M. J., Sofroniew, M. V., Rabbitts, T. H. 1995. The Hox11 gene is essential for cell survival during spleen development. Development 121, 2909-2915.
65. Cope, M., Silvain, C., Khamlichi, A. A., Preud'homme, J. L. 1992. Structurally abnormal immunoglobulins in human immunoproliferative disorders. Blood 79, 2181-2195.
66. Wardemann, H., Boehm, T., Dear, N., Carsetti, R. 2002. B-1a B cells that link the innate and adaptive immune responses are lacking in the absence of the spleen. J. Exp. Med. 195, 771-780.
67. Gary-Gouy, H., Harriaque, J., Bismuth, G., Platzer, C., Schmitt, C., Dalloul, A. H. 2002. Human CD5 promotes B-cell survival through stimulation of autocrine IL-10 production. Blood 100, 4537-4543.
68. Bikah, G., Carey, J., Ciallella, J. R., Tarakhovsky, A., Bondada, S. 1996. CD5-mediated negative regulation of antigen receptor-induced growth signals in B-1 B cells. Science 274, 1906-1909.
69. Tumang, J. R., Hastings, W. D., Bai, C., Rothstein, T. L. 2004. Peritoneal and splenic B-1 cells are separable by phenotypic, functional, and transcriptomic characteristics. Eur. J. Immunol. 34, 2158-2167.
70. Casola, S., Otipoby, K. L., Alimzhanov, M., Humme, S., Uyttersprot, N., Kutok, J. L., Carroll, M. C., Rajewsky, K. 2004. B cell receptor signal strength determines B cell fate. Nat. Immunol. 5, 317-327.
71. Karrer, U., Althage, A., Odermatt, B., Roberts, C. W. M., Korsmeyer, S. J., Miyawaki, S., Hengartner, H., Zinkernagel, R. M. 1997. On the key role of secondary lymphoid organs in antiviral immune responses sudied in alymphoplastic (aly/aly) and spleenless (Hox11^{-/-}) mutant mice. J. Exp. Med. 185, 2157-2170.
72. Meunier, L., Usherwood, Y.-K., Chung, K. T., Hendershot, L. M. 2002. A subset of chaperones and folding enzymes form multiprotein complexes in endoplasmatic reticulum to bind nascent proteins. Mol. Biol. Cell 13, 4465-4469.
73. Mattioli, L., Anelli, T., Fagioli, C., Tacchetti, C., Sitia, R., Valetti, C. 2006. ER storage diseases: a role for ERGIC-53 in controlling the formation and shape of Russell bodies. J. Cell Science 119, 2532-2541.
74. Holmberg, D., Lundkvist, I., Formi, L., Ivars, F., Coutinho, A. 1985. Absence of immunoglobulin heavy chain expression results in altered kappa/lambda L-chain ratios. J. Mol. Cell. Immunol. 2, 51-56.
75. Jamin, C., Lydyard, P. M., Le Corre, R., Youinou, P. Y. 1996. CD5⁺ B cells: differential capping and modulation of IgM and CD5. Scand. J. Immunol. 43, 73-80.
76. Berland, R., Wortis, H. H. 2002. Origins and functions of B-1 cells with notes on the role of CD5. Ann. Rev. Immunol. 20, 253-300.
77. Hardy, R. R. 2006. B-1 B cells: development, selection, natural autoantibody and leukemia. Curr. Opin. Immunol. 18, 547-555.
78. Chen, X., Matsuura, Y., Kearney, J. F. 1995. CD5 transgenic mice. Curr. Top. Microbiol. Immunol. 194, 209-217.
79. Giudicelli, V., Chaume D., Lefranc, M. P. 2004. IMGT/V-QUEST, an integrated software program for immunoglobulin and T cell receptor V-J and V-D-J rearrangement analysis. Nucl. Acids Res. 32, W435-440.
80. Nicholson, I. C., Zou, X., Popov, A. V., Cook, G. P., Corps, E. M., Humphries, S., Ayling, C., Goyenechea, B., Xian, J., Taussig, M. J., Neuberger, M. S., Brüggemann, M. 1999. Antibody repertoires of four- and five-feature translocus mice carrying human immunoglobulin heavy chain and kappa and lambda light chain yeast artificial chromosomes. J. Immunol. 163, 6898-6906.
81. Snoeck V, Peters I R, Cox E. The IgA system: a comparison of structure and function in different species. Vet Res. 2006; 37:455-467.
82. Bastian A, Kratzin H, Eckart K, Hilschmann N. Intra- and interchain disulfide bridges of the human J chain in secretory immunoglobulin A. Biol Chem Hoppe Seyler. 1992; 373:1255-1263.
83. Haas I G, Wabl M. Immunoglobulin heavy chain binding protein. Nature. 1983; 306:387-389.
84. Al-Saleem T, Al-Mondhiry H. Immunoproliferative small intestinal disease (IPSID): a model for mature B-cell neoplasms. Blood. 2005; 105:2274-2280.
85. Seligmann M, Mihaesco E, Preud'homme J L, Danon F, Brouet, J C. Heavy chain diseases: current findings and concepts. Immunol Rev. 1979; 48:145-167.
86. Zou X, Osborn M J, Bolland D J, Smith J A, Corcos D, Hamon M, Oxley D, Hutchings A, Morgan G, Santos F, Kilshaw P J, Taussig M J, Corcoran A E, Brüggemann M. Heavy chain-only antibodies are spontaneously produced in light chain-deficient mice. J Exp Med. 2007; 204:3271-3283.
87. Roux K H, Greenberg A S, Greene L, Strelets L, Avila D, McKinney E C, Flajnik M F. Structural analysis of the nurse shark (new) antigen receptor (NAR): molecular convergence of NAR and unusual mammalian immunoglobulin. Proc Natl Acad Sci USA. 1998; 95:11804-11809.
88. Rumfelt L L, Diaz M, Lohr R L, Mochon E, Flajnik M F. Unprecedented multiplicity of Ig transmembrane and secretory mRNA forms in the cartilaginous fish. J. Immunol. 2004; 173:1129-1139.
89. Litman G W, Cannon J P, Dishaw L J. Reconstructing immune phylogeny: new perspectives. Nat Rev Immunol. 2005; 5:866-879.
90. Kim S, Davis M, Sinn E, Patten P, Hood L. Antibody diversity: Somatic hypermutation of rearranged V_{H} genes. Cell. 1981; 27:573-581.
91. Wagner S D, Neuberger M S. Somatic hypermutation of immunoglobulin genes. Ann Rev Immunol. 1996; 14:441-457.
92. Macpherson A J, Lamarre A, McCoy K, Harriman G R, Odermatt B, Dougan G, Hengartner H, Zinkernagel R M. IgA production without μ or δ chain expression in developing B cells. Nat Immunol. 2001; 2:625-631.
93. Hanson L A, Korotkova M. The role of breastfeeding in prevention of neonatal infection. Semin Neonatol. 2002; 7:275-281.
94. Arakawa H, Iwasato T, Hayashida H, Shimizu A, Honjo T, Yamagishi H. The complete murine immunoglobulin class switch region of the α heavy chain gene-hierarchic repetitive structure and recombination breakpoints. J Biol Chem. 1993; 268:4651-4655.
95. Zou X, Smith J A, Corcos D, Matheson L S, Osborn M J, Brüggemann M. Removal of the BiP-retention domain in Cμ permits surface deposition and developmental progression without L-chain. Mol Immunol. 2008; 45:3573-3579.
96. Pettersson S, Cook G, Brüggemann M, Williams G T, Neuberger M S. A second B-cell specific enhancer is 3′ of the immunoglobulin heavy chain locus. Nature. 1990; 344:165-168.
97. De Genst E, Silence K, Decanniere K, Conrath K, Loris R, Kinne J, Muyldermans S, Wyns L. Molecular basis for the preferential cleft recognition by dromedary heavy-chain antibodies. Proc Natl Acad Sci USA. 2006; 103:4586-4591.
98. Sepúlveda J, Hulin J, Sblattero D, Bradbury A, Burrone O R. Binders based on dimerised immunoglobulin V_{H} domains. J Mol Biol. 2003; 333:355-365.
99. Jin H, Sepúlveda J, Burrone O R. Specific recognition of a dsDNA sequence motif by an immunoglobulin V_{H} homodimer. Prot Science. 2004; 13:3222-3229.
100. Shimoda M, Inoue Y, Ametani A, Fujiwara J, Tsuji N M, Kurisaki J-I, Asuma N, Kanno C. Anti-DNA IgA autoantibodies are spontaneously generated in mouse Peyer's patches. Immunology. 1998; 95:200-207.
101. Kaetzel C S, Robinson J K, Chintalacharuvu K R, Vaerman J P, Lamm M E. The polymeric immunoglobulin receptor (secretory component) mediates transport of immune complexes across epithelial cells: a local defense function for IgA. Proc Natl Acad Sci USA. 1991; 88:8796-8800.
102. Cockle S A, Young N M. The thiol groups of mouse immunoglobulin A: Incomplete formation of the Cα1-domain bridge. Biochem J. 1985:225:113-125.

All documents cited herein are incorporated by reference in their entirety.

We claim:

1. A mouse having functionally silenced endogenous lambda (λ) and kappa (κ) L-chain loci, in which the mouse comprises an antibody-producing cell that produces a H-chain-only antibody lacking a functional C_{H}1 domain following in vivo functional silencing of a gene encoding the C_{H}1 domain.

2. The mouse according to claim 1, having a functionally silenced endogenous heavy chain locus.

3. The mouse according to claim 1, comprising a nucleic acid construct integrated into the endogenous mouse genome, in which the nucleic acid construct comprises non-murine vertebrate heavy chain genes from which a non-murine vertebrate H-chain-only antibody is produced.

4. The mouse according to claim 3, in which the nucleic acid construct comprises one or more mouse C_{H} genes, including a C_{H}1 gene.

5. The mouse according to claim 3, in which the nucleic acid construct excludes a functional non-murine vertebrate C_{H}1 gene.

6. The mouse according to claim 1, in which in vivo functional silencing of the C_{H}1 domain gene is achieved by class switch recombination.

7. The mouse according to claim 1 which is a transgenic mouse having a nucleic acid construct integrated in the endogenous mouse genome, in which the nucleic acid construct comprises non-murine vertebrate V, D and J region genes and in which the mouse produces a mouse-non-murine vertebrate chimeric H-chain-only antibody comprising non-murine vertebrate V, D and J domains and one or more mouse C_{H} domains excluding a functional C_{H}1 domain.

8. The mouse according to claim 7, in which the non-murine vertebrate V, D and J region genes in the construct are in non-murine vertebrate or mouse germline configuration.

9. The mouse according to claim 7, in which the non-murine vertebrate V, D and J domains of the antibody result from recombination in the non-murine vertebrate V, D and J region genes.

10. The mouse according to claim 7, in which the nucleic acid construct is integrated upstream of endogenous mouse C_{H} region genes.

11. The mouse according to claim 7, in which the nucleic acid construct comprises one or more mouse C_{H} region genes including a C_{H}1 domain gene.

12. The mouse according to claim 7, in which the endogenous or nucleic acid construct mouse C_{H}1 domain gene is functionally silenced in vivo in the mouse to allow production of the H-chain-only antibody.

13. The mouse according to claim 12, in which the C_{H}1 domain gene is functionally silenced by class switch recombination.

14. A mouse having functionally silenced endogenous lambda (λ) and kappa (κ) L-chain loci which is a transgenic mouse having a nucleic acid construct integrated in the endogenous mouse genome, in which the nucleic acid construct comprises:
(i) non-murine vertebrate heavy chain V, D, J and C genes, for example in non-murine vertebrate or mouse germline configuration, including a non-murine vertebrate C_{H}1 gene; and
(ii) class switch recombination sequences upstream of the non-murine vertebrate C_{H}1 gene.

15. The mouse according to claim 14, in which the class switch recombination sequences facilitate class switch recombination-mediated functional silencing of the non-murine vertebrate C_{H}1 gene in vivo, thereby allowing production of a non-murine vertebrate H-chain-only antibody in the mouse.

16. The mouse according to claim 14, in which the class switch recombination sequences are murine.

17. The mouse according to claim 3, in which the non-murine vertebrate is a rat or a human.

18. The mouse according to claim 17, in which the vertebrate is a human.

19. The mouse according to claim 14, in which the non-murine vertebrate is a rat or a human.

20. The mouse according to claim 19, in which the vertebrate is a human.

21. An isolated nucleic acid comprising a construct, wherein the construct comprises non-murine vertebrate V, D and J region genes and in which the mouse produces a mouse-non-murine vertebrate chimeric H-chain-only antibody comprising non-murine vertebrate V, D and J domains and one or more mouse C_{H} domains excluding a functional C_{H}1 domain.

22. A host cell comprising the nucleic acid as defined in claim 19.

23. A method for obtaining an H-chain-only antibody from a mouse, comprising the steps of:
(i) producing a mouse with functionally silenced endogenous lambda and kappa L-chain loci;
(ii) allowing formation in the mouse of an H-chain-only antibody lacking a functional C_{H}1 domain following in vivo functional silencing of a gene encoding the C_{H}1 domain; and
(iii) obtaining the H-chain-only antibody from mouse serum.

24. The method according to claim 23, in which the H-chain-only antibody is a non-murine vertebrate antibody or a mouse-non-murine vertebrate chimeric antibody.

25. The method according to claim 24, in which the non-murine vertebrate is human.

26. An isolated antibody-producing cell obtainable using the method as defined in claim 23.

27. A hybridoma obtainable by fusion of an antibody-producing cell as defined in claim 26 with a B-cell tumor line cell.

28. A method for isolating an antibody-producing cell which produces an antigen-specific H-chain-only antibody, comprising the steps of:
(i) obtaining a mouse with functionally silenced endogenous lambda and kappa L-chain loci;
(ii) immunizing the mouse with an antigen;
(iii) selecting for a cell producing an antigen-specific H-chain-only antibody lacking a functional C_{H}1 domain following in vivo functional silencing of a gene encoding the C_{H}1 domain; and
(iv) isolating the cell selected in step (iii).

29. The method according to claim 28, in which the antibody-producing cell is isolated from a secondary lymphoid organ.

30. The method according to claim 29, in which the secondary lymphoid organ is a non-splenic organ, for example any of the group consisting of: lymph node, tonsil, and mucosa-associated lymphoid tissue (MALT), including gut-associated lymphoid tissue (GALT), bronchus-associated lymphoid tissue (BALT), nose-associated lymphoid tissue (NALT), larynx-associated lymphoid tissue (LALT), skin-associated lymphoid tissue (SALT), vascular-associated lymphoid tissue (VALT), and/or conjunctiva-associated lymphoid tissue (CALT).

31. The method according to claim 28, in which the H-chain-only antibody is a non-murine vertebrate antibody or a mouse-non-murine vertebrate chimeric antibody.

32. The method according to claim 31, in which the non-murine vertebrate is human.

33. An isolated antibody-producing cell obtainable using the method as defined in claim 28.

34. A hybridoma obtainable by fusion of an antibody-producing cell as defined in claim 33 with a B-cell tumor line cell.

35. An H-chain-only antibody lacking a functional C_{H}1 domain following in vivo functional silencing of a gene encoding the C_{H}1 domain, or a fragment of the antibody.

36. The antibody according to claim 35, produced in mouse having functionally silenced endogenous lambda and kappa L-chain loci.

37. The antibody according to claim 35, in an isolated and purified form.

38. The antibody according to claim 35, in which the antibody is a monoclonal antibody.

39. An antibody as defined in claim 35 for use as a medicament in the treatment of a disease.

40. An antibody as defined in claim 35 for use in the manufacture of a medicament in the treatment of a disease.

41. A medicament comprising an antibody as defined in claim 35.

42. A method of treating a disease, comprising the step of administering a medicament as a defined in claim 41 to a patient in need of same.

43. The method as defined in claim 42 wherein the disease is selected from the group consisting of wound healing, cell proliferative disorders, including neoplasm, melanoma, lung, colorectal, osteosarcoma, rectal, ovarian, sarcoma, cervical, oesophageal, breast, pancreas, bladder, head and neck and other solid tumors; myeloproliferative disorders, such as leukemia, non-Hodgkin lymphoma, leukopenia, thrombocytopenia, angiogenesis disorder, Kaposis' sarcoma; autoimmune/inflammatory disorders, including allergy, inflammatory bowel disease, arthritis, psoriasis and respiratory tract inflammation, asthma, immunodisorders and organ transplant rejection; cardiovascular and vascular disorders, including hypertension, oedema, angina, atherosclerosis, thrombosis, sepsis, shock, reperfusion injury, and ischemia; neurological disorders including central nervous system disease, Alzheimer's disease, brain injury, amyotrophic lateral sclerosis, and pain; developmental disorders; metabolic disorders including diabetes mellitus, osteoporosis, and obesity, AIDS and renal disease; infections including viral infection, bacterial infection, fungal infection and parasitic infection, pathological conditions associated with the placenta and other pathological conditions.

44. A method for producing an H-chain-only immunoglobulin A (IgA) binding molecule in a mouse, comprising the steps of:
(i) obtaining an L-chain deficient mouse with functionally silenced endogenous lambda and kappa L-chain loci; and
(ii) allowing formation in the L-chain deficient mouse of an H-chain-only IgA binding molecule lacking a functional αC_{H}1 domain.

45. The method according to claim 44, comprising the further step (iii) of isolating the H-chain-only IgA binding molecule.

46. The method according to claim 44, in which the H-chain-only IgA binding molecule is formed following in vivo functional silencing of a gene encoding the αC_{H}1 domain.

47. The method according to claim 46, in which the H-chain-only IgA binding molecule is formed following in vivo deletion of all or a part of the gene encoding the αC_{H}1 domain.

48. The method according to claim 47, in which all or a part of the gene encoding the αC_{H}1 domain is deleted in vivo by imprecise class-switch recombination.

49. The method according to claim 47, in which all or a part of the gene encoding the αC_{H}1 domain is deleted in vivo due to one or more point mutations, out of frame reading, an incorrect stop codon and/or a splice site alteration.

50. The method according to claim 47, in which in vivo deletion of all or a part of the gene encoding the αC_{H}1 domain is not accompanied by DNA insertion.

51. Use of an H-chain-only IgA binding molecule as defined in claim 44 as a screening agent, a diagnostic agent, a prognostic agent, a therapeutic imaging agent, an intracellular binding agent or an abzyme.

52. An H-chain-only IgA binding molecule-producing cell obtainable from an L-chain deficient mouse as defined in claim 44.

53. The cell according to claim 52, which is a bone marrow cell, a mucosal cell or spleen lymphocyte cell.

54. The cell according to claim 53, which is a spleen lymphocyte IgA⁺ B220⁺ cell.

55. An H-chain-only IgA binding molecule-producing hybridoma obtainable by fusion of a B-cell tumor line cell with the cell according to claim 53.

56. A medicament comprising an H-chain-only IgA binding molecule as defined in claim 44.

57. A method of treating a disease, comprising the step of administering a medicament as a defined in claim 56 to a patient in need of same.

58. The method according to claim 57, in which the medicament is administered by the route selected from the group consisting of orally, intramuscularly, intravenously, intradermally, cutaneously, topically, locally, ocularly and inhalation.

59. An isolated nucleic acid encoding an H-chain-only IgA binding molecule as defined in claim 44.

60. A host cell comprising the isolated nucleic acid of claim 59.
